(19) United States
(12) Patent Application Publication
(10) Pub. No.: US 2025/0281523 A1
(43) Pub. Date: Sep. 11, 2025

(54) SULFATED GLYCOSAMINOGLYCAN COMPOUNDS, METHODS, AND USES THEREOF

(71) Applicant: California Institute of Technology, Pasadena, CA (US)

(72) Inventors: Linda C. Hsieh-Wilson, Pasadena, CA (US); Lei Wang, Pasadena, CA (US); Alex Sorum, Pasadena, CA (US); Nitin J. Pawar, Pasadena, CA (US); Huiqian Huang, Pasadena, CA (US)

(21) Appl. No.: 19/076,443

(22) Filed: Mar. 11, 2025

Related U.S. Application Data
(60) Provisional application No. 63/563,874, filed on Mar. 11, 2024.

(51) Int. Cl. A61K 31/737; A61P 25/28; C07H 1/00; C07H 11/00

(52) U.S. Cl. CPC A61K 31/737; A61P 25/28; C07H 1/00; C07H 11/00

(57) ABSTRACT

Provided herein include sulfated glycosaminoglycans including sulfated disaccharides, tetrasaccharides, and oligosaccharides and methods of synthesizing heparan sulfate disaccharide, tetrasaccharide, oligosaccharide library in solution-phase. Provided herein also includes a method of treating neurological diseases or disorders with sulfated glycosaminoglycans such as 4-O-sulfated chondroitin sulfate polysaccharides.

### RELATED APPLICATIONS

This application claims the benefit under 35 U.S.C. § 119(e) of U.S. Provisional Patent Application Ser. No. 63/563,874, filed Mar. 11, 2024, the content of this related application is incorporated herein by reference in its entirety for all purposes.

### STATEMENT REGARDING FEDERALLY SPONSORED R&D

This invention was made with government support under Grant Nos. GM116262, GM134738 and GM093627 awarded by the National Institutes of Health. The government has certain rights in the invention.

### BACKGROUND

The present disclosure relates generally to the field of glycosaminoglycans, and particularly sulfated glycosaminoglycans.

### DESCRIPTION OF THE RELATED ART

Glycosaminoglycans (GAGs) are abundant, ubiquitous carbohydrates in biology, yet their structural complexity has limited an understanding of their biological roles and structure-function relationships. Synthetic access to large collections of well defined, structurally diverse GAG oligosaccharides would provide critical insights into this important class of biomolecules and represent a major advance in glycoscience.

### SUMMARY

Disclosed herein include sulfated glycosaminoglycans, and related methods for the synthesis of the compounds. Also disclosed include use of the compounds disclosed herein and compositions thereof for treating diseases such as immune diseases, and neurological diseases and disorders.

In some embodiments, a disaccharide is disclosed as well as methods for synthesizing the disaccharide. The disaccharide can have a formula of:

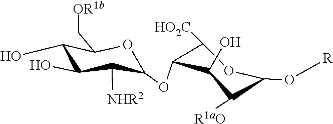

wherein R^{1a}, R^{1b} and R² are independently H, methyl, ethyl, acetyl (Ac), SO₃H, PO2(OCH₃)H or PO₃H₂; and R is H, a C1-C12 1 R group linear or branched, substituted or unsubstituted aliphatic primary or secondary amine, a C1-C12 linear or branched, substituted or unsubstituted aliphatic primary or secondary amine. The R can be a fluorous tag. In some embodiments, R^{1a} and/or R^{1b} is a hydroxyl protecting group. In some embodiments, the disaccharide is N-sulfated and/or O-sulfated. In some embodiments, R² is SO₃H, i^{1a} is SO₃H, and/or R^{1b} is SO₃H.

Disclosed herein also includes a polysaccharide as well as methods for synthesizing the polysaccharide. The polysaccharide can have a formula of

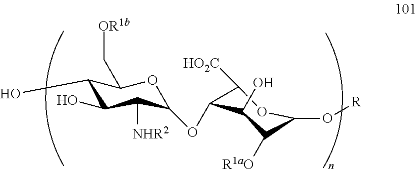

wherein n is any integer ranging from 2 to 200; R^{1a}, R^{1b} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine. In some embodiments, wherein R^{1a}, R^{1b} and/or R² is SO₃H.

Disclosed herein also includes a polysaccharide as well as methods for synthesizing the polysaccharide. The polysaccharide can have a formula of

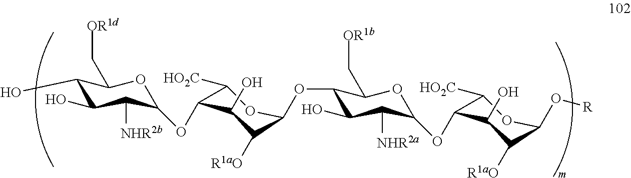

wherein m is any integer ranging from 1 to 100; R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine. In some embodiments, R^{1a}, R^{1b}, R^{1c}, R^{1d}, R^{2a}, and/or R^{2b} is SO₃H.

Disclosed herein also includes a method of treating a neurological disease or disorder in a subject in need thereof. In some embodiments, the method can comprise administrating to the subject a therapeutically effective amount of a composition comprising an effective amount of a 4-O-sulfated chondroitin sulfate polysaccharide, thereby treating the neurological disease or disorder in the subject.

In some embodiments, the 4-O-sulfated chondroitin sulfate polysaccharide has a general formula of:

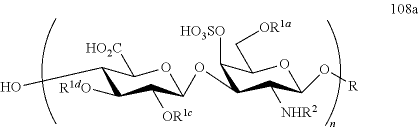

wherein n is any integer ranging from 2 to 200, preferably 20-200; R^{1a}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine. In some embodiments, the 4-O-sulfated chondroitin sulfate polysaccharide has a general formula of:

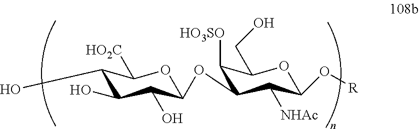

wherein R is H, a C1-C12 linear or branched, substituted or unsubstituted aliphatic primary or secondary amine; n is any integer ranging from 20 to 200. In some embodiments, the 4-O-sulfated chondroitin sulfate polysaccharide has a general formula of:

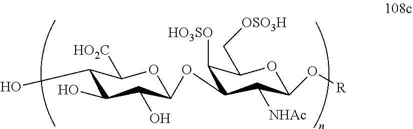

wherein R is H, a C1-C12 linear or branched, substituted or unsubstituted aliphatic primary or secondary amine; n is any integer ranging from 20 to 200.

In some embodiments, the neurological disease or disorder is selected from the group consisting of: Alzheimer's disease, bipolar disorder, schizophrenia, autism, fragile X syndrome, Rett syndrome, anxiety or anxiety-related disorder, social memory dysfunction, and a combination thereof. In some embodiments, the subject is a human. In some embodiments, the composition is administered to the subject by intravenous administration.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1 depicts an exemplary embodiment illustrating the synthesis of a comprehensive HS tetrasaccharide library. a, 64-compound library representing all possible N-, 2-O- and 6-O-sulfate modifications of the tetrasaccharide GlcN-IdoA-GlcN-IdoA. b, Universal building block for HS library synthesis. The six orthogonal functional groups are shown in colour. c, General strategy and late-stage modification steps. IdoA, 1-iduronic acid; GlcN, d-glucosamine; GlcNAc, N-acetyl-d-glucosamine; Fmoc, 9-fluorenylmethoxycarbonyl; Lev, levulinoyl; TBDPS, tert-butyldiphenylsilyl; Nap, 6-O-(2-naphthyl)methyl; TFA, trifluoroacetyl; Bz, benzoyl; Bn, benzyl.

FIG. 2 depicts an exemplary embodiment illustrating the synthesis of universal building block 65. Preparation of orthogonally protected tetrasaccharide 65 starts with acidic hydrolysis of natural heparin to form a core disaccharide, which is converted to versatile intermediate 68 in nine steps. Installation of the fluorous tag (69 to 70) and subsequent protecting-group manipulations give disaccharide acceptor 72. TFAA, trifluoroacetic anhydride; Im, imidazole. Conversion of key intermediate 68 along another route yields disaccharide donor 74, which, upon glycosylation with 72, affords universal building block 65 in good yield.

FIG. 3 depicts an exemplary embodiment illustrating the divergent synthesis of the HS tetrasaccharide library. a, Strategy for the synthesis of libraries 2OH(1) and 2OS(1) from 65. b, Synthesis of the 16-compound NS(2)-2OS(1) sub-library. Reagents and conditions: ^{a}DDQ, DCE, MeOH, PBS, room temperature (r.t.); ^{b}hydrazine acetate, MeOH, DCM, r.t.; ^{c}HF·Py, py, 0° C. to r.t.; ^{d}SO₃·Et₃N, DMF, 50° C.; ^{e}1 M LiOH, MeOH, THF, 40° C.; ^{f}Ac₂O, Et₃N, MeOH, r.t.; ^{g}PMe₃, NaOH, THF, r.t.; ^{h}SO₃·Py, NaOH, Et₃N, TFE, MeOH, r.t.; ⁱPd(OH)₂, H₂, t-BuOH, H₂O, r.t.

FIG. 4 depicts non-limiting exemplary data showing FGF2 recognizes specific HS sulfation sequences. a, Representative fluorescence image of FGF2 binding to the HS tetrasaccharide microarray. Compound numbers are indicated on the array. b, Heatmap of the relative binding of FGF2 to each member of the 64-compound library. Binding signals were normalized with respect to the compound with greatest binding. The tetrasaccharide backbone structure is schematically represented with sites of modification labelled by ‘X.’ The error is the standard deviation to the first digit of uncertainty for the mean across nine replicates. c, The sulfation logo of FGF2. d, Selected tetrasaccharides highlight the regiospecific contributions of N-sulfation and 2-O-sulfation to FGF2 binding.

FIG. 5 is a table comparing binding of selected glycans to FGF2. The shown glycans facilitate systematic analysis of the importance of individual sulfate modifications to FGF2 binding.

FIG. 6 depicts HS sulfation specificities of FGF4 and CXCL8. a, Heatmap of the relative binding of FGF4 to the 64-compound HS library. Binding signals were normalized with respect to the compound with greatest binding. Error is the standard deviation to the first digit of uncertainty for the mean across nine replicates. b, Sulfation logo for FGF4. c, Heatmap of the relative binding of CXCL8. Error is the standard deviation to the first digit of uncertainty for the mean across nine replicates. d, Sulfation logo for CXCL8.

FIG. 7 illustrates an alternative synthetic route to tetrasaccharide 76.

FIG. 8 illustrates the synthesis of HS sub-library NS(2)-2OS(1). Reagents and conditions: (a) DDQ, DCE, MeOH, PBS, rt. (b) hydrazine acetate, MeOH, DCM, rt. (c) HF·Py, pyridine, 0° C. to rt. (d) SO₃·Et₃N, DMF, 50° C. (e) 1 M LiOH, MeOH, THF, 40° C. (f) Ac₂O, TEA, MeOH, rt. (g) PMe₃, NaOH, THF, rt. (h) SO₃·Py, NaOH, TEA, TFE, MeOH, rt. (i) Pd(OH)₂, H₂, t-BuOH, H₂O, rt.

FIG. 9 illustrates the synthesis of HS sub-library NAc(2)-2OS(1). Reagents and conditions: (a) DDQ, DCE, MeOH, PBS, rt. (b) hydrazine acetate, MeOH, DCM, rt. (c) HF·Py, pyridine, 0° C. to rt. (d) SO₃·Et₃N, DMF, 50° C. (e) 1 M LiOH, MeOH, THF, 40° C. (f) Ac2O, TEA, MeOH, rt. (g) SO₃·Py, NaOH, TEA, TFE, MeOH, rt. (h) Pd(OH)₂, H₂, t-BuOH, H₂O, rt. (i) AcSH, pyridine, rt. 16 h.

FIG. 10 depicts synthesis of the C₆F₁₃ linker S56 and C₈F₁₇ linker S57 (a) and synthesis of tetrasaccharide S63 with the C₈F₁₇ linker (b).

FIG. 11 depicts the comparison between the C₆F₁₃ and C₈F₁₇ F-tags.

FIG. 12 depicts synthesis of HS sub-library NS(2)-2OH(1) and compounds 57-58. Reagents and conditions: (a) DDQ, DCE, MeOH, PBS, rt. (b) hydrazine acetate, MeOH, DCM, rt. (c) HF·Py, pyridine, 0° C. to rt. (d) SO₃·Et₃N, DMF, 50° C. (e) 1 M LiOH, MeOH, THF, rt. (f) Ac₂O, TEA, MeOH, 40° C. (g) PMe₃, NaOH, THF, rt. (h) SO3·Py, NaOH, TEA, TFE, MeOH, rt. (i) Pd(OH)₂, H₂, t-BuOH, H₂O, rt.

FIG. 13 depicts synthesis of HS sub-library NAc(2)-2OH(1). (a) DDQ, DCE, MeOH, PBS, rt. (b) hydrazine acetate, MeOH, DCM, rt. (c) HF·Py, pyridine, 0° C. to rt. (d) SO₃·Et₃N, DMF, 50° C. (e) 1 M LiOH, MeOH, THF, 40° C. (f) Ac₂O, TEA, MeOH, rt. (g) SO₃·Py, NaOH, TEA, TFE, MeOH, rt. (h) Pd(OH)₂, H₂, t-BuOH, H₂O, rt. (i) AcSH, pyridine, rt, 16 h.

FIG. 14 is a table showing structures and purities of HS tetrasaccharides 1-64.

FIG. 15 displays non-limiting exemplary data depicting relative binding of FGF2, FGF4 and CXCL8 (from top to bottom) to the tetrasaccharide arrays. The fluorescence intensities were corrected for background and normalized with respect to the highest intensity on the array. Bar graphs represent the mean±SD for each compound in nonduplicate. Individual data points are shown as red dots.

FIG. 16 is a table showing structures, relative binding, and standard deviation (s.d.) of HS tetrasaccharides to FGF2, FGF4, and CXCL8.

FIG. 17 depicts chemical formulas, chemical names, and abbreviations of HS tetrasaccharides 1-64.

FIG. 18 depicts an automation platform for HS synthesis. a, Schematic illustration of that automation platform and reaction cycle. b, Automated solution-phase synthesis of HS tetrasaccharides 94 and 95.

FIG. 19 depicts automated synthesis of compounds 94 and 95 (panel a) and automated deprotection of the Nap group (panel b).

FIG. 20 depicts divergent synthesis of HS disaccharide library. a) HF·Py, Py, 0° C. to rt. b) hydrazine acetate, DCM, MeOH, rt. c) PMe₃, NaOH, THF, rt. d) Ac₂O, Et₃N, MeOH, rt. e) SO₃·Et₃N, DMF, 50° C. f) 1 M LiOH, MeOH, THF, 40° C. g) S03 Py, NaOH, Et₃N, TFE, MeOH, rt. h) Pd(OH)₂, H₂, t-BuOH, H₂O, rt.

FIG. 21 displays non-limiting exemplary results depicting that Chst11cKO mice lacking chondroitin 4-O-sulfation have a striking increase in PNNs surrounding excitatory neurons in the CA2 hippocampus. (A) Biosynthetic pathways leading to 4-O- and 6-O-sulfation of chondroitin sulfate (CS). CS consists of the repeating disaccharide unit N-acetyl-D-galactosamine-β(1,3)-D-glucuronic acid. n=20 to 200. In the 4-O-sulfation pathway (orange, studied in this work), the disaccharide units are monosulfated at the 4-O-position of GalNAc to generate CS-A. CS-A is subsequently sulfated at the 6-O-position of GalNAc to produce CS-E. In the 6-O-sulfation pathway (blue), the disaccharide units are monosulfated at the 6-O-position of GalNAc to generate CS-C, which is subsequently sulfated at the 2-O-position of GlcA to form CS-D. (B) Disaccharide analysis shows loss of the CS-A and CS-E motifs in the brains of Chst11cKO mice. Amounts of CS-A and CS-E in the cortex of Ctrl (blue) and Chst11cKO (red) mice were quantified at postnatal day (P) 0, P7, P14, P28, and P60 by dividing the number of picomoles (pmol) of CS by the total weight (mg) of the dried cortex homogenate. (C) Representative images showing PNN-enwrapped (WFA⁺, green), PV⁺ interneurons (red), and PCP4⁺ excitatory pyramidal neurons (blue) in the hippocampus of Ctrl and Chst11cKO mice. Hippocampal CA1, CA2, and CA3 regions are marked with dotted white rectangles. (Scale bar, 500 μm.) Quantification shows the number of WFA⁺ neurons in each region. ***P<0.001, ****P<0.0001 vs. Ctrl, Student's t test; n=17 slices from 7 Ctrl mice and 14 slices from 5 Chst11cKO mice. (D) Magnified images showing the CA2 regions of Ctrl and Chst11cKO mice, stained for PCP4 (blue), PNNs (WFA, green), and PV (red). (Scale bar, 50 μm.) Quantification of the ratios of PV⁺ neurons (Upper) and WFA⁺ neurons (Lower) among PCP4+CA2 neurons. ****P<0.0001 vs. Ctrl, Student's t test; n=10 and 8 slices from 4 pairs of Ctrl and Chst11cKO mice, respectively. All data are shown as the mean±SEM.

FIG. 22 displays non-limiting exemplary results depicting impaired synapse development in Chst11cKO hippocampal neurons. (A) Ctrl and Chst11cKO mice were injected with GFP-expressing lentivirus into the hippocampal regions. Representative images showing dendritic spines of viral-infected CA1 and CA2 neurons. Mature spines are indicated by white arrowheads. (Scale bar, 5 μm.) (B and C) CA1 neurons from Chst11cKO mice display fewer mature spines with smaller head widths compared to Ctrl mice. Quantification of the mature spine density is shown in (B). **P<0.01 vs. Ctrl, Student's t test. The cumulative distribution curve of spine head widths is shown in (C). ****P<0.0001 vs. Ctrl, Kolmogorov-Smirnov test. n=23 and 22 dendrites from 4 pairs of Ctrl and Chst11cKO mice, respectively. (D and E) CA2 neurons from Chst11cKO mice display fewer mature spines with similar head widths compared to Ctrl mice. Quantification of the mature spine density is shown in (D). ****P<0.0001 vs. Ctrl, Student's t test. Cumulative distribution curve of spine head widths is shown in (E). n=13 and 16 dendrites from 4 pairs of Ctrl and Chst11cKO mice, respectively. (F and G) Hippocampal neurons from Chst11cKO mice have fewer excitatory synapses and more inhibitory synapses compared to Ctrl mice. Representative images and quantification of PSD-95 puncta (red) distributed along the dendrites (MAP2, blue) of Ctrl and Chst11cKO neurons are shown in (F). Representative images and quantification of gephyrin puncta (red) of Ctrl and Chst11cKO neurons are shown in (G). Dendrites were traced with dotted lines. (Scale bar, 10 μm.) ****P<0.0001 vs. Ctrl, Student's t test; n=20 neurons each for Ctrl and Chst11cKO mice. All data are shown as the mean±SEM.

FIG. 23 displays non-limiting exemplary results depicting that modulation of PNN density or CS 4-O-sulfation levels regulates hippocampal synapses. (A) ChABC treatment rescued the decreased density of excitatory synapses in Chst11cKO neurons. Representative images and quantification of PSD-95 puncta (red) distributed along the dendrites (MAP2, blue) of the indicated cultured neurons. (Scale bar, 10 μm.) ****P<0.0001 vs. Chst11cKO, one-way ANOVA followed by Tukey's multiple comparisons test; n=10, 11, and 10 neurons for Ctrl, Chst11cKO, and ChABC-treated Chst11cKO conditions, respectively. (B) ChABC treatment rescued the increased density of inhibitory synapses in Chst11cKO neurons. Representative images and quantification of gephyrin puncta (red) distributed along the dendrites (MAP2, blue) of the indicated cultured neurons. (Scale bar, 10 μm.) ****P<0.0001 vs. Chst11cKO, one-way ANOVA followed by Tukey's multiple comparisons test; n=10 neurons per condition. (C) CS-A or CS-E, but not CS-C, polysaccharides rescued the decreased density of excitatory synapses in Chst11cKO neurons. Representative images and quantification of PSD-95 puncta (red) distributed along the dendrites (MAP2, blue) of the indicated cultured neurons. (Scale bar, 10 μm.) Quantification of PSD-95 puncta number per 10 μm. ****P<0.0001 vs. Chst11cKO, one-way ANOVA followed by Tukey's multiple comparisons test; n=15, 13, 14, 10, and 12 neurons for Ctrl, Chst11cKO, and Chst11cKO neurons treated with CS-A, CS-E or CS-C, respectively. (D) A chemical inhibitor of the CS-E sulfotransferase Chst15 (Inhib, 10 μM in DMSO, 24 h) elevated PNN levels (Top), decreased the density of excitatory synapses (Middle) and increased the density of inhibitory synapses (Bottom). Hippocampal neurons were cultured from wild-type C57 mice. DMSO was used as a control (Con). Quantification of the number of WFA⁺ neurons (normalized to Con) is shown in (D). **P<0.01 vs. Con, Student's t test; n=7 regions each for cultured neurons treated with Con or Inhib. Representative images and quantification of both PSD-95 and gephyrin puncta distributed along the dendrites (MAP2, blue) of the indicated cultured neurons are also shown in (D). (Scale bar, 10 μm.) ***P<0.001, ****P<0.0001 vs. Con, Student's t test. For PSD-95, n=9 and 10 neurons treated with Con or Inhib, respectively. For gephyrin, n=8 and 9 neurons treated with Con or Inhib, respectively. All data are shown as the mean±SEM.

FIG. 24 displays non-limiting exemplary results depicting that reduced p-CREB levels in Chst11cKO hippocampal neurons can be rescued by treatment with ChABC or 4-O-sulfated CS polysaccharides. (A) Representative images showing hippocampal slices of Ctrl and Chst11cKO mice stained with a phospho-Ser133 CREB antibody (red) and WFA (green). The Left panels show images of the CA2 and CA3 regions. (Scale bar, 200 μm.) The Right panels show the cropped and magnified images of the CA1, CA2, and CA3 regions used for quantifications. (Scale bar, 50 μm.) (B) Quantification of the average p-CREB fluorescence intensity per neuron in the indicated hippocampal regions (normalized to Ctrl CA1 region). *P<0.05 vs. Ctrl, Student's t test; n=6 pairs of Ctrl and Chst11cKO mice. (C) ChABC or penicillinase (Pen) was injected bilaterally into hippocampal CA2 regions. Representative images of Ctrl and Chst11cKO hippocampal slices 2 wk after injection. Slices were stained with WFA (green) and an anti-PCP4 antibody (blue; lower panels in gray scale). (Scale bar, 500 μm.) (D) Quantification of the average p-CREB fluorescence intensity per neuron (normalized to Pen-treated Ctrl mice) in the CA2 region for each condition.**P<0.01, ****P<0.0001, two-way ANOVA, Genotype×Treatment: F(1,33)=1.181, P=0.2850 for CA2; n=10, 9, 9, and 9 slices from 3 mice each for Ctrl Pen, Ctrl ChABC, Chst11cKO Pen, and Chst11cKO ChABC conditions, respectively. (E) Quantification of p-CREB levels for Ctrl, Chst11cKO, and ChABC-treated Chst11cKO neuronal cultures (normalized to Ctrl). ****P<0.0001 vs. Chst11cKO, one-way ANOVA followed by Tukey's multiple comparisons test; n=300 to 400 neurons for each condition. (F) Treatment with 4-O-sulfated CS-A or CS-E polysaccharides, but not 6-O-sulfated CS-C polysaccharides, rescued the p-CREB levels in Chst11cKO neurons. Quantification of p-CREB levels for Ctrl, Chst11cKO, and CS polysaccharide-treated (20 μg/mL, 24 h) Chst11cKO neurons. *P<0.05, ****P<0.0001 vs. Chst11cKO, one-way ANOVA followed by Tukey's multiple comparisons test; n=˜100 to 150 neurons for each condition. All data are shown as the mean±SEM.

FIG. 25 displays non-limiting exemplary results depicting that loss of CS 4-O-sulfation and high PNN densities in the area CA2 impair social memory and increase anxiety. (A) Schematic depicting the two-trial social memory test employed in this study. (B and C) Chst11cKO mice exhibit impairments in social memory that are not observed in Ctrl mice. Interaction times are shown for Ctrl mice (B, **P<0.01 vs. trial 1, paired Student's t test; n=9) or Chst11cKO mice (C, not significant; n=11) injected with Pen. Each circle represents an individual subject. (D-F) Injection of ChABC into the CA2 region rescued the social memory deficits in Chst11cKO mice and impaired social memory in Ctrl mice. The average ratio of the 2nd to the 1st interaction time for each condition is shown in (D). **P<0.01, two-way ANOVA, Genotype×Treatment: F(1,36)=29.90, P<0.0001; n=9, 11, 10, and 10 mice for Ctrl Pen, Chst11cKO Pen, Ctrl ChABC, and Chst11cKO ChABC groups, respectively. Interaction times are shown for Ctrl mice (E, not significant; n=10) or Chst11cKO mice (F, ***P<0.001 vs. trial 1, paired Student's t test; n=10) injected with ChABC. (G-I) Chst11cKO mice exhibit elevated anxiety levels compared to Ctrl mice. The average percentage of time spent in the center of the arena during the OFT is shown in (G). **P<0.01 vs. Ctrl, Student's t test; n=12 Ctrl and 10 Chst11cKO mice. The average percentage of time spent in the open arms of the EPM is shown in (H). *P<0.05 vs. Ctrl, Student's t test; n=10 Ctrl and 11 Chst11cKO mice. The average percentage of time spent in the lighted box of the LDB is shown in (1). *P<0.05 vs. Ctrl, Student's t test; n=10 Ctrl and 11 Chst11cKO mice. (J-L) PNN digestion in the CA2 region restored the anxiety levels of Chst11cKO mice back to Ctrl levels, as shown in the OFT and LDB tests. For the OFT (J), **P<0.01 vs. Chst11cKO Pen, two-way ANOVA, Genotype×Treatment: F(1,54)=3.253, P=0.0769; n=13, 13, 16, and 16 mice for Ctrl Pen, Chst11cKO Pen, Ctrl ChABC, and Chst11cKO ChABC groups, respectively. For the EPM (K), *P<0.05, ***P<0.001 vs. Ctrl Pen, two-way ANOVA, Genotype×Treatment: F(1,48)=11.32, P=0.0015; n=13, 12, 13, and 14 mice for Ctrl Pen, Chst11cKO Pen, Ctrl ChABC, and Chst11cKO ChABC groups, respectively. For the LDB (L), *P<0.05, **P<0.01 vs. Chst11cKO Pen, two-way ANOVA, Genotype×Treatment: F(1,52)=11.75, P=0.0012; n=14, 13, 15, and 14 mice for Ctrl Pen, Chst11cKO Pen, Ctrl ChABC, and Chst11cKO ChABC groups, respectively. All data are shown as the mean±SEM.

FIG. 26 displays non-limiting exemplary results depicting that CA2-specific deletion of Chst11 increases PNN densities and impairs social memory. (A) CA2 region-specific Chst11 deletion using AAV-CaMKIIα-mCherry-Cre (Cre) results in increased PNN densities. Representative images of the CA2 region from Cre-injected or AAV-CamKIIα-mCherry (Con)-injected mice are shown. Hippocampal slices were labeled with mCherry (red), anti-PCP4 antibody (blue), and WFA (green). (Scale bar, 50 μm.) Quantification of the PNN density, measured as the number of WFA⁺ neurons in the CA2 region (normalized to Con). ****P<0.0001 vs. Con, Student's t test; n=10 brain slices each from 4 pairs of Cre- and Con-injected mice. (B) Reduced p-CREB levels in the CA2 region of Cre-injected floxed Chst11 mice. Representative images showing the CA2 regions of Con- and Cre-injected floxed Chst11 mice, stained with p-CREB antibody (gray scale in separate image; blue in merged image), mCherry (red), and WFA (green). Quantification of p-CREB levels in the CA2 regions of Cre-injected floxed Chst11 mice and Con-injected mice. *P<0.05 vs. Con, Student's t test; n=12 slices each from 4 pairs of Con- and Cre-injected animals. (C-E) Cre-injected mice displayed impaired social memory compared to Con-injected mice. The ratio of the 2nd to the 1st interaction time for Con- and Cre-injected mice is shown in (C). *P<0.05 vs. Con, Student's t test; n=10 and 9 for Con-injected and Cre-injected mice, respectively. Interaction times for Con-injected mice (D, **P<0.01 vs. trial 1, paired Student's t test; n=10) and Cre-injected mice (E, not significant; n=9). Each circle represents an individual subject. All data are shown as the mean±SEM.

FIG. 27 displays non-limiting exemplary results depicting disruption of the 4-O-sulfation pathway in Chst11cKO mice. (A) HPLC disaccharide analysis was used to quantify the amounts of CS-C, CS-D, unsulfated CS and total CS in the cortex of Ctrl (blue) and Chst11cKO (red) mice at postnatal day (P) 0, P7, P14, P28 and P60. The amounts were calculated by dividing the number of picomoles (pmol) of CS by the total weight (mg) of the dried homogenate. (B) Representative images of brain slices showing the visual cortex (VC) of Ctrl and Chst11cKO mice immunostained with an anti-CS-E monoclonal antibody. Scale bars, 100 μm. (C) Representative images of brain slices showing the hippocampus (Hpc) of Ctrl and Chst11cKO mice immunostained with a CS-E monoclonal antibody. Scale bars, 500 μm.

FIG. 28 displays non-limiting exemplary results depicting increased PNN densities in the VC of Chst11cKO mice. (A) Representative images showing PNN-enwrapped (WFA⁺) and parvalbumin-expressing (PV⁺) neurons in the VC of Ctrl and Chst11cKO mice. Brain sections were stained with WFA (green), an anti-parvalbumin antibody (red), and DAPI (blue). Scale bar, 100 μm. (B) Quantification of the number of WFA+ neurons in the VC. **P<0.01 vs. Ctrl, Student's t-test. (C) Percentage of WFA⁺ PV⁺ neurons amongst the PV+ neurons. ***P<0.001 vs. Ctrl, Student's 1-test. (D) Quantification of the number of PV⁺ neurons in the VC. Not significant. (E) Percentage of WFA⁺ PV⁺ neurons amongst the WFA⁺ neurons. Not significant. n=14 brain slices each from 4 pairs of Ctrl and Chst11cKO mice for B-E. All data are shown as the mean±SEM.

FIG. 29 displays non-limiting exemplary results depicting comparable aggrecan levels in the CA2 regions of Ctrl and Chst11cKO mice. (A) Representative images showing PNN and aggrecan staining in the hippocampus of Ctrl and Chst11cKO mice. Brain sections were stained with WFA (green), an anti-aggrecan antibody (red), and DAPI (blue). The CA2 regions are marked with dotted white squares. Scale bar, 500 μm. n=3 pairs each of Ctrl and Chst11cKO mice. (B) Magnified images showing the PNN-enriched CA2 regions of Ctrl and Chst11cKO mice, stained for aggrecan (red) and PNNs (WFA, green). Scale bar, 50 sm.

FIG. 30 displays non-limiting exemplary results depicting low, comparable levels of Otx2 in the CA2 regions of Ctrl and Chst11cKO mice. (A) Representative images showing PNN and Otx2 staining in the hippocampus of Ctrl and Chst11cKO mice. Brain sections were stained with WFA (green), an anti-parvalbumin antibody (red), and an anti-Otx2 antibody (grey in separate images; blue in merged images). CA2 regions are marked with dotted white squares. Scale bar, 500 μm. n=2 pairs each of Ctrl and Chst11cKO mice. (B) Magnified images showing the PNN-enriched CA2 regions of Ctrl and Chst11cKO mice, stained for Otx2 (grey scale), PNNs (WFA, green), and PV (red). Scale bar, 50 μm.

FIG. 31 displays non-limiting exemplary results depicting dendritic spine morphology of hippocampal neurons in Ctrl and Chst11cKO mice. (A) Representative images showing the hippocampal CA1, CA2, and CA3 regions of Ctrl mice injected with GFP-expressing lentivirus. Scale bar, 100 μm. (B to D) No significant difference was observed in the protrusion densities in the CA1 (B), CA2 (C), and CA3 (D) regions of Chst11cKO mice compared to Ctrl mice. Not significant; n=23, 22 dendrites in the CA1, n=13, 16 dendrites in the CA2, and n=11, 15 dendrites in the CA3 regions of Ctrl and Chst11cKO mice, respectively. Data are shown as the mean±SEM. (E to G) CA3 neurons from Chst11cKO mice have larger dendritic spine head widths, but similar mature spine density. Representative images of the dendritic spines from GFP-expressing CA3 neurons are shown in E. Scale bar, 5 μm. Quantification of the mature spine density is shown in F. Not significant; n=11 and 15 dendrites from 3 pairs of Ctrl and Chst11cKO mice, respectively. Data are shown as the mean±SEM. Cumulative distribution curves of spine head width for Ctrl and Chst11cKO CA3 neurons are shown in G. ****P<0.0001 vs. Ctrl, Kolmogorov-Smirnov test.

FIG. 32 displays non-limiting exemplary results depicting altered PNN densities and synapse sizes in cultured hippocampal Chst11cKO neurons compared to Ctrl neurons. (A) Schematic showing the collection and culturing of hippocampal neurons from individual embryos at embryonic day 15 (E15). The tail of each embryo was collected for genotyping. (B) Representative images showing hippocampal neuron cultures from Ctrl and Chst11cKO embryos stained with WFA (green) and an anti-MAP2 antibody (grey scale). The same PNN⁺ neurons were indicated with white or red arrowheads in separate channels for WFA or MAP2, respectively. Scale bar, 100 sm. (C) Quantification of the number of WFA⁺ neurons per mm2. ****P<0.0001 vs. Ctrl, Student's 1-test; n=22 and 21 regions of cultured neurons from 3 pairs of Ctrl and Chst11cKO mice, respectively. (D) Quantification of PSD-95 puncta size. ****P<0.0001 vs. Ctrl, Student's t-test; n=20 neurons each for Ctrl and Chst11cKO mice. (E) Quantification of gephyrin puncta size. ***P<0.001 vs. Ctrl, Student's 1-test; n=20 neurons each for Ctrl and Chst11cKO mice. All data are shown as the mean±SEM.

FIG. 33 displays non-limiting exemplary results depicting ChABC treatment of cultured Chst11cKO neurons removes excess PNNs and restores synapse sizes to Ctrl neuron levels. (A) Representative images showing wild-type C57 hippocampal neurons treated with PBS (Control) or ChABC for 2, 8, or 24 h, and stained for PNNs (WFA, green) and MAP2(grey scale). Scale bar, 100 μm. (B) Representative images of Ctrl, Chst11cKO, and ChABC-treated Chst11cKO cultured hippocampal neurons stained for PNNs (WFA, green) and MAP2 (grey scale). The same PNN+ neurons were indicated with white or red arrow heads in separate channels for WFA or MAP2, respectively. Scale bar, 100 μm. (C) Quantification of the number of WFA+ neurons in each visual field, normalized to Ctrl. *P<0.05, ***P<0.001, ****P<0.0001 vs. Ctrl, one-way ANOVA followed by Tukey's multiple comparisons test; n=6, 6, and 8 regions of Ctrl, Chst11cKO, and ChABC-treated Chst11cKO cultured neurons, respectively. (D) ChABC treatment of Chst11cKO neurons restored the PSD-95 puncta size to Ctrl neuron levels. ****P<0.0001 vs. Ctrl, one-way ANOVA followed by Tukey's multiple comparisons test; n=10, 11, and 10 Ctrl, Chst11cKO, and ChABC-treated Chst11cKO cultured neurons, respectively. (E) ChABC treatment of Chst11cKO neurons restored the gephyrin puncta size to Ctrl neuron levels. ****P<0.0001 vs. Ctrl, one-way ANOVA followed by Tukey's multiple comparisons test; n=10 neurons for each condition. All data are shown as the mean±SEM.

FIG. 34 displays non-limiting exemplary results depicting chemical manipulation of CS 4-O-sulfation levels modulates PNN densities and synapses. (A and B) Treatment of Chst11cKO neurons (13-15 DIV) with natural CS polysaccharides (20 μg/ml) enriched in the 4-O-sulfated CS-A or CS-E motifs, but not the 6-O sulfated CS-C motif, restored PNN densities to Ctrl neuron levels. Representative images of hippocampal neuronal cultures for the conditions indicated are shown in A. Scale bar, 100 μm. Quantification of the number of WFA⁺ neurons per mm2 is shown in B. **P<0.01, ***P<0.001 vs. Chst11cKO, one-way ANOVA followed by Tukey's multiple comparisons test; n=9, 8, 9, 9, and 9 regions of Ctrl, Chst11cKO, and CS-A, CS-E or CS-C-treated Chst11cKO cultured neurons, respectively. (C to E) Addition of natural CS polysaccharides to cultured Chst11cKO hippocampal neurons did not rescue the increased density or size of gephyrin puncta. Representative images of gephyrin puncta (red) distributed along the dendrites of neurons (MAP2, blue) cultured under the indicated conditions are shown in C. Scale bar, 10 μm. Quantification of gephyrin puncta number per 10 μm (D) and size (E). **P<0.01, ***P<0.001 vs. Chst11cKO, one-way ANOVA followed by Tukey's multiple comparisons test; n=10, 12, 10, 10, and 10 for Ctrl, Chst11cKO, and CS-A, CSE or CS-C-treated Chst11cKO neurons, respectively. (F) Addition of natural CS polysaccharides enriched in the CS-A or CS-E motifs, but not the CS-C motif, rescued the decreased size of PSD-95 puncta in Chst11cKO neurons. Quantification shows PSD-95 puncta size. **P<0.01, ***P<0.001 vs. Chst11cKO, one-way ANOVA followed by Tukey's multiple comparisons test; n=15, 13, 14, 10, and 12 neurons for Ctrl, Chst11cKO, and Chst11cKO neurons treated with CS-A, CS-E or CS-C, respectively. (G) Treatment with a sulfotransferase inhibitor (Inhib, 10 μM, 24 h) reduced the PSD-95 puncta size. Quantification of PSD-95 puncta size. ***P<0.001 vs. Con, Student's t-test; n=10 and 9 neurons treated with Inhib in DMSO or DMSO alone (Con), respectively. (H) Inhib treatment (10 μM, 24 h) did not change the gephyrin puncta size. Quantification of gephyrin puncta size. n=9 and 8 neurons treated with Inhib in DMSO or DMSO alone (Con), respectively. All data are shown as the mean±SEM.

FIG. 35 displays non-limiting exemplary results depicting mIPSC and mEPSC recordings of CA2 neurons from Ctrl and Chst11cKO mice. (A and B) Raw traces of mIPSC (A) and mEPSC (B) of CA2 neurons from Ctrl and Chst11cKO mice. (C and D) Quantification revealed that mIPSC frequency (C) for Chst11cKO CA2 neurons showed an increasing trend compared to Ctrl CA2 neurons. P=0.07, Wilcoxon rank-sum one-sided test. The mIPSC amplitude (D) was not significantly different between Ctrl and Chst11cKO neurons; n=14 and 15 neurons from 3 Ctrl and 5 Chst11cKO mice, respectively. (E and F) mEPSC frequency (E) and amplitude (F) were comparable for Ctrl and Chst11cKO CA2 neurons. Not significant; n=14 neurons each from 4 Ctrl and 6 Chst11cKO mice. All data are shown as the mean±SEM.

FIG. 36 displays non-limiting exemplary results depicting PNN densities modulate CREB phosphorylation levels, but not total CREB levels, in the hippocampus. (A) Representative images showing the CA1, CA2, and CA3 regions of the hippocampus stained with a p-CREB antibody (red) and WFA (green). Each region was cropped with a rectangular box, as indicated, for analysis of p-CREB levels at the cellular level. Scale bar, 500 μm. (B) Representative images showing the CA2 and CA3 hippocampal regions of Ctrl and Chst11cKO mice immunostained for CREB (red). Scale bar, 200 μm. (C) Representative images of cropped CA1, CA2, and CA3 regions of total CREB. Scale bar, 50 sm. (D and E) Quantification of the average p-CREB fluorescence intensity per neuron (normalized to Ctrl Pen) in the hippocampal CA1 (D) and CA3 (E) regions of Ctrl and Chst11cKO mice, injected with ChABC or penicillinase (Pen). *P<0.05, two-way ANOVA, F(1,33)=0.7723, P=0.3859 for CA3; n=10, 9, 9, and 9 slices from 3 mice each for Ctrl Con, Ctrl ChABC, Chst11cKO Con, and Chst11cKO ChABC conditions, respectively. All data are shown as the mean±SEM.

FIG. 37 displays non-limiting exemplary results depicting modulation of PNN density and CS 4-0 sulfation levels affects p-CREB levels in hippocampal neurons. (A) Representative images of cultured hippocampal neurons stained with WFA (green) and anti-p-CREB or anti-CREB antibodies (red) as indicated. WFA⁺ neurons are marked with a yellow arrowhead. Scale bar, 25 μm. (B) Quantification of p-CREB and CREB intensities, normalized to WFA⁻ as a control. *P<0.05 vs. WFA−, Student's 1-test; n=11 and 8 individual WFA⁻ and WFA⁺ neurons stained with p-CREB, respectively; n=8 and 5 individual WFA⁻ and WFA⁺ neurons stained for CREB, respectively. (C) Quantification of p-CREB and CREB intensities in neurons treated with ChABC for 2, 8 and 24 h. **P<0.01, ****P<0.0001 vs. Con, one-way ANOVA followed by Tukey's multiple comparisons test; n=100-150 neurons for each condition. (D) Representative images showing Ctrl and Chst11cKO hippocampal neurons treated with ChABC or PBS as a control and stained with an anti-p-CREB antibody (upper panels, grey scale; lower panels, red) and AF488 phalloidin (green). Scale bar, 50 μm. (E) Quantification of total CREB levels for Ctrl, Chst11cKO, and ChABC-treated Chst11cKO neurons (normalized to Ctrl). **P<0.01 vs. Chst11cKO, one-way ANOVA followed by Tukey's multiple comparisons test; n=100-150 neurons for each condition. (F) Quantification of total CREB levels for Ctrl, Chst11cKO, and CS-A-, CS-E- and CS-C-treated (20 μg/ml, 24 h) Chst11cKO neurons, respectively. **P<0.01 vs. Chst11cKO, one-way ANOVA followed by Tukey's multiple comparisons test; n=˜100-150 neurons for each condition. All data are shown as the mean±SEM.

FIG. 38 displays non-limiting exemplary results depicting locomotor activity of Ctrl and Chst11cKO mice is comparable and unaffected by ChABC treatment. (A) Quantification of the total distance traveled during each OFT trial. Not significant; n=12 Ctrl and 10 Chst11cKO mice. (B) Quantification of the total distance traveled in the EPM. Not significant; n=10 Ctrl and 11 Chst11cKO mice. (C) Quantification of the total distance traveled by Ctrl and Chst11cKO mice in the OFT during each trial. Animals were injected with ChABC or Pen as a control. Not significant; n=13, 13, 16, and 16 mice for the Ctrl Pen, Chst11cKO Pen, Ctrl ChABC, and Chst11cKO ChABC conditions, respectively. (D) Quantification of the total distance traveled by Ctrl and Chst11cKO mice in the EPM. Not significant; n=13, 12, 13, and 14 mice for the Ctrl Pen, Chst11cKO Pen, Ctrl ChABC, and Chst11cKO ChABC conditions, respectively. All data are shown as the mean±SEM.

FIG. 39 displays non-limiting exemplary results depicting specific deletion of 4-O-sulfation in CA2 pyramidal neurons does not affect anxiety-like behavior. (A) Reduced 4-O-sulfation in CA2 neurons infected with AAV-CaMKIIα-mCherry-Cre (Cre). Representative images showing the hippocampal CA2 regions of floxed Chst11 mice injected with AAV-CamKIIα-mCherry (Con) or AAV-CaMKIIα-mCherry-Cre (Cre). Brain sections were labeled with an anti-PCP4 antibody (blue), anti-CS-E antibody (green), and mCherry (red). Scale bar, 50 μm. (B to D) Cre-injected animals did not exhibit changes in anxiety like behavior. Percentage of time spent in the center of the arena during the OFT (B). Percentage of time spent in the open arms of the EPM (C). Percentage of time spent in the light box of the LDB (D). n=10 pairs of Con- and Cre-injected mice for the OFT. n=9 and 10 pairs of Con- and Cre-injected mice, respectively, for the EPM and LDB tests. All data are shown as the mean±SEM.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Glycosaminoglycans (GAGs), also known as mucopolysaccharides, are negatively-charged polysaccharide compounds and are composed of repeating disaccharide units that are present in mammalian tissue. The four primary groups of GAGs are classified based on their core disaccharide units and include heparin/heparan sulfate, chondroitin sulfate/dermatan sulfate, keratan sulfate, and hyaluronic acid. The interaction between GAGs and proteins can have profound physiological effects on hemostasis, lipid transport and adsorption, cell growth and migration and development

Heparan sulfate glycosaminoglycans or heparan sulfate (HS) are ubiquitous, complex polysaccharides that mediate diverse biological processes, ranging from cell proliferation and viral invasion to neural development and immune regulation. Their linear polysaccharide chains are composed of repeating disaccharides that undergo sulfation at various positions, resulting in an enormous number of different sulfation sequences. The sulfation motifs enable HS to interact with more than 2,000 proteins and thus are central to their biological activities. Moreover, alterations in HS sulfation patterns are associated with human diseases such as cancer, Alzheimer's disease, osteoarthritis and cardiovascular diseases. However, an in-depth understanding of their physiological and pathological roles has been hampered by limited access to defined HS structures. Large collections of HS oligosaccharides representing a diversity of sulfation motifs will be essential for elucidating the structure-function relationships of HS and expanding the development of GAG-based therapeutics.

As HS GAGs are among the most structurally complex carbohydrates in nature, their chemical synthesis remains an immense challenge. For example, HS can display 16 distinct sulfation motifs within its disaccharide unit alone. Its synthesis requires regioselective differentiation of five hydroxyls and one amino group within each disaccharide, stereoselective glycosylation of low-reactivity glycosyl donors and acceptors, access to 1-iduronic acid (IdoA) monosaccharides that are not commercially available and are tedious to prepare, and multi-step purifications of highly charged, polar intermediates. As such, the synthesis of HS oligosaccharides is notoriously difficult and labour-intensive, requiring specialized expertise in carbohydrate chemistry. For example, a single HS tetrasaccharide typically takes 35-50 chemical steps depending on the sulfation pattern. Thus, accessing large libraries of sulfated HS oligosaccharides represents a formidable barrier. Indeed, comprehensive GAG libraries representing all possible sulfation patterns have not been achieved except in the case of HS disaccharides. Despite key synthetic advances and elegant targeted syntheses, the majority of HS sequence space remains both chemically and biologically unexplored.

In response to these challenges, efficient platforms have been developed to accelerate the synthesis of GAGs. Chemical methods have focused primarily on automating glycan assembly and on less structurally complex GAGs such as chondroitin sulfate (CS), keratan sulfate (KS) and hyaluronan rather than on generating large libraries of sulfated GAGs. Moreover, the automated synthesis of HS oligosaccharides has not yet been reported, presumably due to challenges with the additional late-stage modification steps (for example, N-sulfation). Thus, the development of new synthetic platforms for rapid GAG production, particularly the synthesis of large HS oligosaccharide libraries, remains an important, unrealized goal. Such platforms are much needed to provide broad access to a wide range of glycan structures, similar to the now routine production of peptides and oligonucleotides.

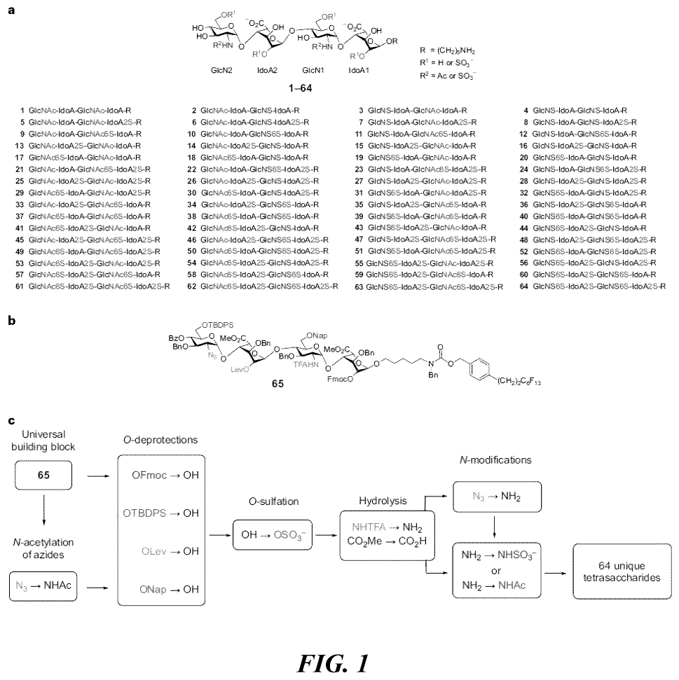

Provided herein includes a method for the expedient solution-phase synthesis of sulfated glycosaminoglycans (e.g., HS GAGs) displaying comprehensive arrays of diverse sulfation motifs. Disaccharide synthons derived from natural heparin are employed and a universal tetrasaccharide building block is designed to substantially reduce the total number of synthetic steps. Diversification of this building block provided a comprehensive library of 64 sulfated tetrasaccharides (1-64; FIG. 1A) representing all possible products from differential sulfation of six functional groups (the 2-O-, 6-O- and N-positions) within the tetrasaccharide. To simplify the late-stage modification steps required for HS library synthesis, a traceless, fluorous tagging method is developed to allow for rapid purification of the highly charged, sulfated intermediates by fluorous solid-phase extraction (FSPE). The comprehensive HS library enabled systematic investigations into the unique sulfation dependencies of HS-binding proteins and identified position-dependent, sequence-specific modifications critical for HS recognition by growth factors and chemokines important for morphogenesis, cell growth and inflammatory responses. HS compounds disclosed herein can bind to growth factors and chemokines, and be used to agonize (activate) or antagonized (inactivate) those proteins for treating diseases such as, inflammation, immune diseases, neurological diseases, and cancer.

Disclosed herein include heparan sulfate (HS) disaccharide, tetrasaccharide and oligosaccharide compound libraries synthesized using the method described herein. Disclosed herein also include chondroitin sulfate disaccharide, tetrasaccharide and polysaccharide compound libraries.

In some embodiments, a disaccharide can have a formula of

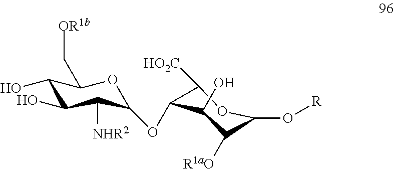

wherein R^{1a}, R^{1b} and R² are independently H, methyl, ethyl, acetyl (Ac), SO₃H, PO2(OCH₃)H or PO₃H₂; and R is H, a C1-C12 1 R group linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a} and/or R^{1b} is a hydroxyl protecting group.

Disclosed herein also includes a polysaccharide having a

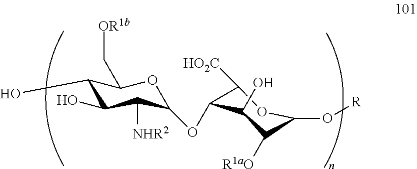

wherein n is any integer ranging from 2 to 200; R^{1a}, R^{1b} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

Disclosed herein also includes a method of treating neurological disease or disorder using sulfated glycosaminoglycans. In some embodiment, the method can comprise administrating to the subject a therapeutically effective amount of a composition comprising an effective amount of a 4-O-sulfated chondroitin sulfate polysaccharide, thereby treating the neurological disease or disorder in the subject. In some embodiments, the 4-O-sulfated chondroitin sulfate polysaccharide has a general formula of 108a, 108b or 108c. In some embodiments, the neurological disease or disorder is Alzheimer's disease, bipolar disorder, schizophrenia, autism, fragile X syndrome, Rett syndrome, anxiety or anxiety-related disorder, and/or social memory dysfunction.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

The term “polysaccharide” as used herein indicates a polymeric saccharide having different lengths and dimensions. In particular, polysaccharides in the sense of the disclosure encompass polymeric carbohydrate molecules composed of chains of monosaccharide units bound together by glycosidic linkages, which on hydrolysis give the constituent monosaccharides and/or oligosaccharides. A “glycosidic linkage” as used herein indicates an oxygen atom that joins an anomeric carbon atom of one monosaccharide to a designated carbon atom on an adjacent monosaccharide and is typically designed as an (L) as will be understood by a skilled person. The glycosidic linkage is denoted by α or β (m→n), wherein α or β represents a configuration of an anomeric carbon m of a monosaccharide and n represents the corresponding carbon on an adjacent monosaccharide to which the glycosidic linkage is connected. Polysaccharide in the sense of the disclosure range in structure from linear to highly branched and are often quite heterogeneous, containing slight modifications of the repeating unit. Polysaccharides, have a general formula of Cₓ(H₂O)_{y} where x can be a number between 200 or lower and 2500 or higher. The term “oligosaccharide” refers to a polymeric saccharide having a few sugars, typically 3-15 linked together with glycosidic bonds.

The term “disaccharide” as used herein refers to a moiety having two monosaccharide units joined by a glycosidic linkage. The term “tetrasaccharide” as used herein refers to a moiety having four monosaccharide units joined sequentially by a glycosidic linkage.

The terms “alkyl” refer to a straight or branched chain saturated hydrocarbon moiety. including but not limited to C₁-C₁₀ straight-chain alkyl groups or C₁-C₁₀ branched-chain alkyl groups. In some embodiments, the “alkyl” group refers to C₁-C₆ straight-chain alkyl groups or C₁-C₆ branched-chain alkyl groups. In some embodiments, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “alkenyl” refers to a straight or branched chain hydrocarbon moiety having one or more carbon-carbon double bonds. In one embodiment, the alkenyl group contains 1, 2, or 3 double bonds and is otherwise saturated. In some embodiments, the “alkenyl” group contains from 2 to 24 carbon atoms. Alkenyl groups include both cis and trans isomers. Representative straight chain and branched alkenyl groups include ethylenyl, propylenyl, 1-butenyl, 2-butenyl, isobutylenyl, 1-pentenyl, 2-pentenyl, 3-methyl-1-butenyl, 2-methyl-2-butenyl, and 2,3-dimethyl-2-butenyl.

The term “alkynyl” refers to a straight or branched chain hydrocarbon moiety having one or more carbon-carbon triple bonds. In some embodiments, the “alkynyl” group contains from 2 to 24 carbon atoms. Representative straight chain and branched alkynyl groups include acetylenyl, propynyl, 1-butynyl, 2-butynyl, 1-pentynyl, 2-pentynyl, and 3-methyl-1-butynyl.

The terms “branched alkyl”, “branched alkenyl”, and “branched alkynyl” refer to an alkyl, alkenyl, or alkynyl group in which one carbon atom in the group (1) is bound to at least three other carbon atoms and (2) is not a ring atom of a cyclic group. For example, a spirocyclic group in an alkyl, alkenyl, or alkynyl group is not considered a point of branching.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfuydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O-.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “hydroxyalkyl” refers to an alkyl group having a hydroxyl attached thereto. Representative hydroxyalkyl groups include hydroxyethyl (—CH₂CH₂OH), hydroxypropyl, hydroxybutyl, hydroxypentyl, and the like.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

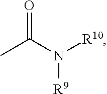

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. If an amide is drawn without depicting a group required by valence on the constituent nitrogen atom (e.g., as in —C(O)—NR⁹), then a hydrogen atom is implied and understood to be present on the nitrogen atom to satisfy the aforementioned valence requirement (e.g., as in —C(O)—NHR⁹).

The terms “amine” and “amino” are art-recognized and refer to both unsubstituted and substituted amines and salts thereof, e.g., a moiety that can be represented by

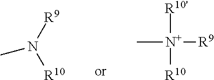

wherein R⁹, R¹⁰, and R¹⁰ each independently represent a hydrogen or a hydrocarbyl group, or R⁹ and R¹⁰ taken together with the N atom to which they are attached complete a heterocycle having from 4 to 8 atoms in the ring structure.

The term “amino alkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

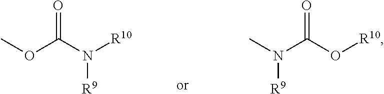

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bi cyclic rings. Each ring of a bi cyclic carbocycle may be selected from saturated, unsaturated and aromatic rings. Carbocycle includes bi cyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O-. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “sulfate” is art-recognized and refers to the group —OSO₃H, or a pharmaceutically acceptable salt thereof.

The term “sulfonamide” is art-recognized and refers to the group represented by the general formula:

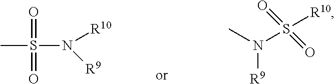

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “thioalkyl”, as used herein, refers to an alkyl group substituted with a thiol group.

The term “thioester”, as used herein, refers to a group —C(O)SR⁹ or —SC(O)R⁹, wherein R⁹ represents a hydrocarbyl.

The term “thioether”, as used herein, is equivalent to an ether, wherein the oxygen is replaced with a sulfur.

The term “urea” is art-recognized and may be represented by the general formula

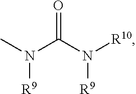

wherein R⁹ and R¹⁰ independently represent hydrogen or a hydrocarbyl.

The term “bind”, “binding”, “conjugation” as used herein indicates an attractive interaction between two elements which results in a stable association of the element in which the elements are in close proximity to each other. If each element is comprised in a molecule the result of binding is typically formation of a molecular complex. Attractive interactions in the sense of the present disclosure includes both non-covalent binding and, covalent binding. Non-covalent binding as used herein indicates a type of chemical bond, such as protein protein interaction, that does not involve the sharing of pairs of electrons, but rather involves more dispersed variations of electromagnetic interactions. Non-covalent bonding includes ionic bonds, hydrophobic interactions, electrostatic interactions, hydrogen bonds, and dipole-dipole bonds. Electrostatic interactions include association between two oppositely charged entities. An example of an electrostatic interaction includes using a charged lipid as the functional membrane lipid and binding an oppositely charged target molecule through electrostatic interactions.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented. A treatment is considered “effective treatment” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

As used herein, the terms “effective amount” or “pharmaceutically effective amount” or “therapeutically effective amount” refer to an amount sufficient to effect beneficial or desirable biological and/or clinical results.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipients can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. In some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human. In some embodiments, the subject has or is suspected of having a neurological disease or disorder.

### Chemical Synthesis and an Automated Synthesis Platform

Disclosed herein also includes a method of synthesizing sulfated glycosaminoglycans (GAGs) such as heparan sulfate (HS) and chondroitin sulfate (CS) including disaccharides, tetrasaccharides, and oligosaccharides. Disclosed herein also includes a novel automated solution-phase synthesis method for heparan sulfate (HS) oligosaccharide libraries encompassing a diverse range of sulfation motifs. Existing methods for automated glycosaminoglycan (GAG) synthesis have relied on solid supports, which require excess reagents and often suffer from solvent incompatibility and limited scalability. Moreover, the synthesis of GAGs is notoriously challenging, often requiring 40-50 steps for each oligosaccharide. As such, new strategies for the solution-phase, automated synthesis of glycans, particularly large GAG oligosaccharide libraries, represent an important advance.

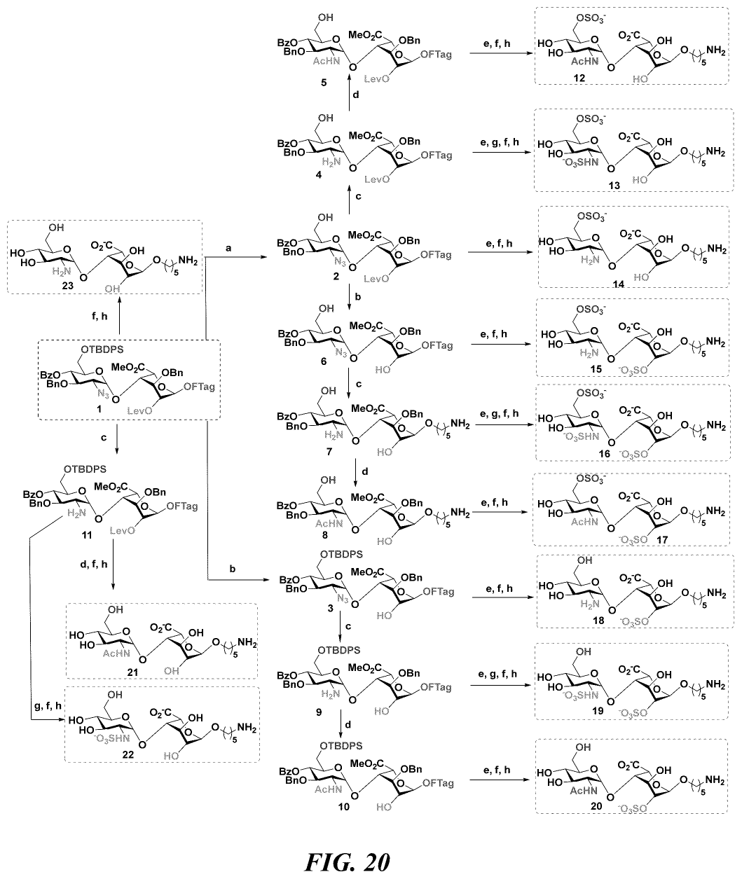

The method presented herein provides a short, highly efficient route which differs from traditional methods that typically employ monosaccharide building blocks and utilize manual synthesis (e.g., Org. Lett. 2006, 8, 26, 5995-5998). By instead employing disaccharide synthons obtained through controlled acidic hydrolysis of natural heparin, the total number of chemical steps required is reduced by more than one-half. The synthesis route also eliminates the need to synthesize an L-iduronic acid acceptor and circumvents the challenges of selective 1,2-cis glycosylation in the D-glucosamine donor. Notably, a universal building block (1) with four orthogonal protecting groups was developed to distinguish specific hydroxyl functionalities, thereby significantly streamlining the overall synthetic process (FIG. 20). Diversification of universal building block 1 enabled the construction of a comprehensive library of 12 HS disaccharides, encompassing all possible 2-O-, 6-O-, and N-sulfation patterns (compounds 12-23, FIG. 20). To enhance the efficiency of the process further, a traceless fluorous tagging strategy was implemented, which avoided the laborious, multi-step purifications of the highly charged, sulfated intermediates, enabling their rapid purification via fluorous solid-phase extraction (FSPE). Methods were developed to perform this FSPE purification on the ChemSpeed automation platform, ultimately affording the HS disaccharides in high purity and good overall yield.

The synthesis begins with the selective unmasking of orthogonal protecting groups, followed by O-sulfation to introduce sulfate groups at the targeted 2-O- and 6-O-positions. Subsequent treatment with LiOH (e.g., at 1M) saponifies the methyl esters, after which Staudinger reduction converts the azide into an amine. At this stage, the amino group can be left alone or further modified by either N-sulfation or N-acetylation. Following these different amino group modifications, global deprotection via hydrogenation removes the benzyl groups, yielding the desired disaccharide analogs. This approach demonstrates how a single universal building block can be leveraged to efficiently generate a diverse library of HS disaccharides using our novel solution-phase automated synthesis platform. The HS libraries including HS disaccharides, tetrasaccharides, and oligosaccharides, can be expanded to include other sulfation motifs and longer oligosaccharide lengths.

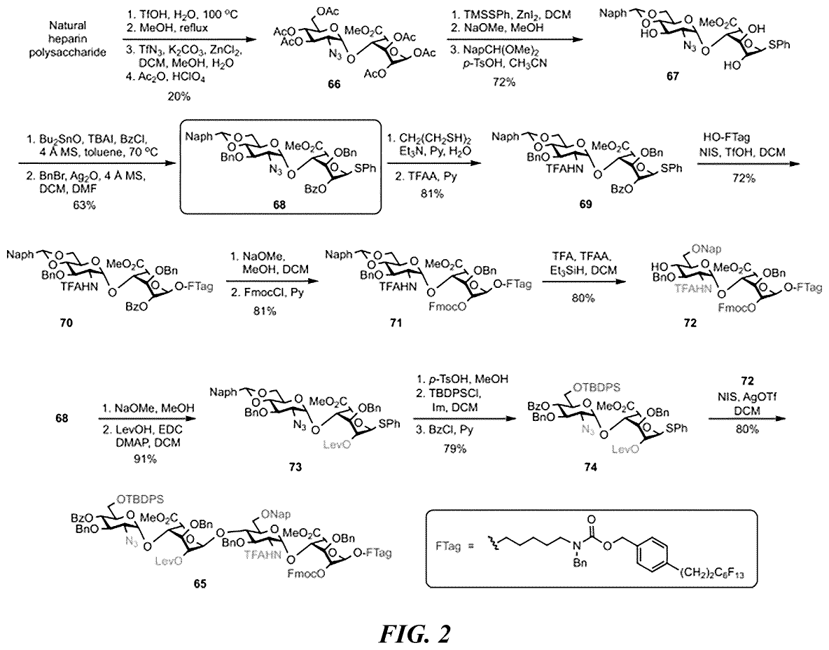

The exemplary embodiment shown in FIG. 20 illustrates the divergent synthesis of HS disaccharide library. The exemplary embodiment shown in Example 3 and FIG. 2 describes the synthesis of the orthogonally protected tetrasaccharide building block based on the use of natural heparin. Preparation of orthogonally protected tetrasaccharide (65) starts with acidic hydrolysis of natural heparin to form a core disaccharide, which is converted to versatile intermediate (68). Installation of the fluorous tag (69 to 70) and subsequent protecting group manipulations give disaccharide acceptor 72. Conversion of key intermediate 68 along another route yields disaccharide donor 74, which, upon glycosylation with 72, affords universal building block 65 in good yield.

### Heparan Sulfate (HS) Glycosaminoglycans JX

Provided herein are HS glycosaminoglycans (GAGs) and HS disaccharide, tetrasaccharide, and oligosaccharide compound libraries synthesized using the methods described herein. This comprehensive library of HS disaccharide, tetrasaccharide, oligosaccharides contain a variety of 2-O, 6-O- and N-sulfation sequences and represents a diverse collection of sulfated HS GAGs synthesized.

Heparan sulfate (HS) is a member of glycosaminoglycan (GAG) family of carbohydrates and is structurally related to heparin. HS is a linear polysaccharide composed of a variety of sulfated repeating disaccharide units of alternating N-substituted glucosamine and hexuronic acid (glucuronic or iduronic acid). The most common disaccharide unit within heparin sulfate is a glucuronic acid (GlcA) linked to N-acetylglucosamine (GlcNAc). In some embodiments, heparin sulfate described herein comprise GlcA, GlcNAc, and/or L-iduronic acid (IdoA) with all possible 2-O-, 6-O- and N-sulfation. HS is typically found in the form of heparan sulfate proteoglycans (HSPGs), which are glycoproteins composed of HS chains. HSPGs are found on the cell surface, basement membrane, and extracellular matrix, and can participate in a variety of activities at the local level, including cell migration, cell adhesion, synaptogenesis and as receptors for cytokines. HSPGs are present in the basement membrane where they interact with other matrix components to establish the structure of the basement membrane and to provide cell migration routes. HSPGs can function as receptors for proteases and protease inhibitors, thereby regulating their distribution and activity. Membrane proteoglycans interact with cell adhesion receptors to facilitate cell-ECM attachment, cell-cell interactions, and cell motility. HSPGs can also bind chemokines, cytokines, growth factors, and morphogens, thereby protecting them from proteolysis. This also facilitates the formation of morphogen gradients essential for cell specification during development.

The HS disaccharides, tetrasaccharides and oligosaccharides described herein can comprise any one of the monosaccharides described herein. A monosaccharides can have anomeric carbon present in α or β isomeric form. Monosaccharides 97a, 97b, are D-glucosamine in α, β forms respectively, while 97c represents D-glucosamine in either α or β form. Monosaccharides 98a, 98b, are L-Iduronic acid in α, β forms respectively, while 98c represents L-Iduronic acid in either α or β form. Monosaccharides 9a, 99b are D-glucuronic acid in β, α forms respectively, while 99c represents L-Iduronic acid in either α or β form. Oligosaccharides can be made based on any combination of these monosaccharide isomers with different substitutions including those from differential sulfation of six functional groups (the 2-O-, 6-O- and N-positions). Other substitution or derivatization includes but is not limited to methylation, ethylation, acetylation (Ac), sulfation (SO₃H), phosphation (PO2(OCH₃)H or PO₃H₂) and functionalization with C1-C12 linear or branched, substituted or unsubstituted aliphatic primary or secondary amine, a C1-C12 linear or branched, substituted or unsubstituted aliphatic primary or secondary amine. The oligosaccharides as described herein include any permutation of the monosaccharides (97a to 99c) in any substitution or derivatization forms. In some embodiments, a glycosidic bond is formed between an α or β nomeric carbon of one monosaccharide and a hydroxyl group of another monosaccharide moiety in an oligosaccharide as described herein.

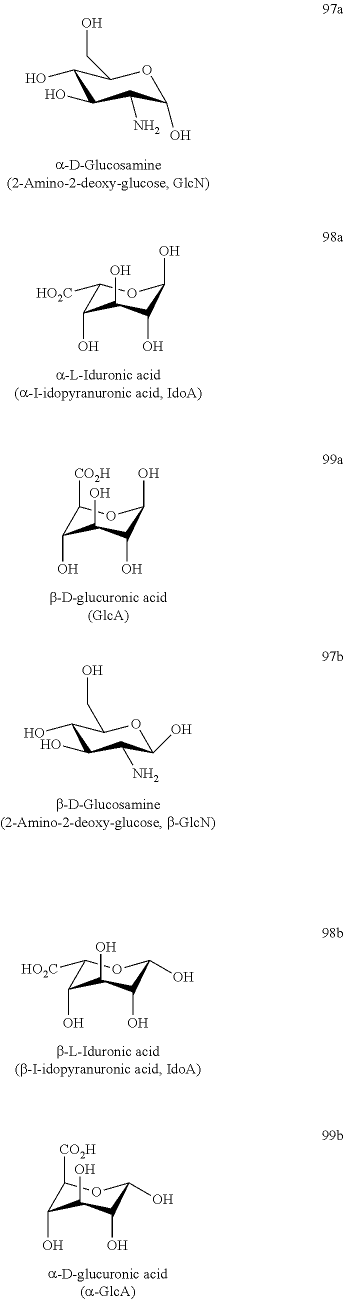

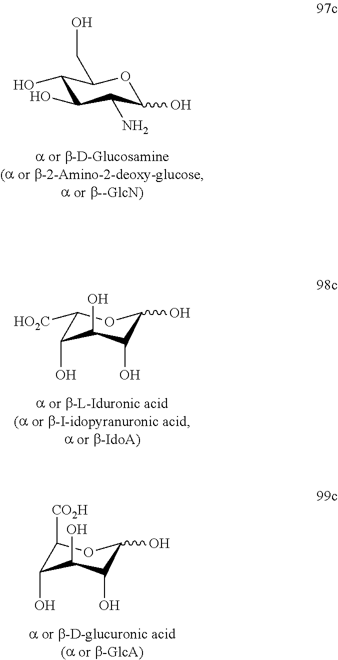

Disclosed herein includes a HS disaccharide such as GlcN-GlcA disaccharide. In some embodiments, GlcN-GlcA disaccharides are represented by formula 95:

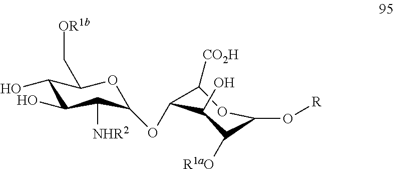

wherein R^{1a}, R^{1b} and R² are independently H, methyl, ethyl, acetyl (Ac), SO₃H, PO₂(OCH₃)H or PO₃H₂; and R is H, a C1-C12 1 R group linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a}, R^{1b} is a hydroxyl protecting group.

In some embodiments, a GlcN-GlcA disaccharide is represented by formula 95, wherein R² is SO₃H. In some embodiments, R group of the GlcN-GlcA disaccharide of formula 95 is a C1-C12 linear aliphatic primary amine. In some embodiments, R group of the GlcN-GlcA disaccharide of formula 95 is a C2-C8 linear aliphatic primary amine. In some embodiments, R group of the GlcN-GlcA disaccharide of formula 95 is a C5 linear aliphatic primary amine (—(CH₂)₅NH₂).

In some embodiments, a GlcN-GlcA disaccharide is represented by formula 95, wherein R² is an acetyl group (Ac). In some embodiments, R group of the GlcN-GlcA disaccharide of formula 95 is a C1-C12 linear aliphatic primary amine. In some embodiments, R group of the GlcN-GlcA disaccharide of formula 95 is a C2-C8 linear aliphatic primary amine. In some embodiments, R group of the GlcN-GlcA disaccharide of formula 95 is a C5 linear aliphatic primary amine (—(CH₂)₅NH₂).

In some embodiments, in an O-sulfated GlcN-GlcA disaccharide is represented by formula 95, wherein R^{1a} and R^{1b} are H or SO₃H with the proviso that at least one of R^{1a} and R^{1b} is SO₃H. In some embodiments, an O-sulfated GlcN-GlcA disaccharide is represented by formula 95, wherein R^{1a} and R^{1b} are SO₃H. In some embodiments, R group of the O-sulfated GlcN-GlcA disaccharide of formula 95 is a C1-C12 linear aliphatic primary amine. In some embodiments, R group of GlcN-GlcA disaccharide of formula 95 is a C2-C8 linear aliphatic primary amine. In some embodiments, R group of the O-sulfated GlcN-GlcA disaccharide of formula 95 is a C5 linear aliphatic primary amine (—(CH₂)₅NH₂).

In some embodiments, the N-sulfated, O-sulfated GlcN-GlcA disaccharide is represented by formula 95, wherein R² is SO₃H, and wherein R^{1a} and R^{1b} are SO₃H.

In some embodiments, R group of the GlcN-GlcA disaccharide of formula 95 is a C1-C12 linear aliphatic primary amine. In some embodiments, R group of the GlcN-GlcA disaccharide of formula 95 is a C2-C8 linear aliphatic primary amine. In some embodiments, R group of GlcN-GlcA disaccharide of formula 95 is a C5 linear aliphatic primary amine (—(CH₂)₅NH₂).

Disclosed herein includes a HS disaccharide such as GlcN-IdoA disaccharide. In some embodiments, GlcN-IdoA disaccharides are represented by formula 96:

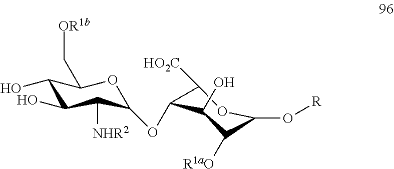

wherein R^{1a}, R^{1b} and R² are independently H, methyl, ethyl, acetyl (Ac), SO₃H, PO₂(OCH₃)H or PO₃H₂; and R is H, a C1-C12 1 R group linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a}, R^{1b} is a hydroxyl protecting group.

In some embodiments, a GlcN-IdoA disaccharide is represented by formula 96, wherein R² is SO₃H. In some embodiments, R group of the GlcN-IdoA disaccharide of formula 96 is a C1-C12 linear aliphatic primary amine. In some embodiments, R group of the GlcN-IdoA disaccharide of formula 96 is a C2-C8 linear aliphatic primary amine. In some embodiments, R group of the GlcN-IdoA disaccharide of formula 96 is a C5 linear aliphatic primary amine (—(CH₂)₅NH₂).

In some embodiments, a GlcN-IdoA disaccharide is represented by formula 96, wherein R² is an acetyl group (Ac). In some embodiments, R group of the GlcN-IdoA disaccharide of formula 96 is a C1-C12 linear aliphatic primary amine. In some embodiments, R group of the GlcN-IdoA disaccharide of formula 96 is a C2-C8 linear aliphatic primary amine. In some embodiments, R group of the GlcN-IdoA disaccharide of formula 96 is a C5 linear aliphatic primary amine (—(CH₂)₅NH₂).

In some embodiments, in an O-sulfated GlcN-IdoA disaccharide is represented by formula 96, wherein R^{1a} and R^{1b} are H or SO₃H with the proviso that at least one of R^{1a} and R^{1b} is SO₃H. In some embodiments, an O-sulfated GlcN-IdoA disaccharide is represented by formula 96, wherein R^{1a} and R^{1b} are SO₃H. In some embodiments, R group of the O-sulfated GlcN-IdoA disaccharide of formula 96 is a C1-C12 linear aliphatic primary amine. In some embodiments, R group of GlcN-IdoA disaccharide of formula 96 is a C2-C8 linear aliphatic primary amine. In some embodiments, R group of the O-sulfated GlcN-IdoA disaccharide of formula 96 is a C5 linear aliphatic primary amine (—(CH₂)₅NH₂).

In some embodiments, the N-sulfated, O-sulfated GlcN-IdoA disaccharide is represented by formula 96, wherein R² is SO₃H, R^{1a} is SO₃H, and/or R^{1b} are SO₃H.

In some embodiments, R group of the GlcN-IdoA disaccharide of formula 96 is a C1-C12 linear aliphatic primary amine. In some embodiments, R group of the GlcN-IdoA disaccharide of formula 96 is a C2-C8 linear aliphatic primary amine. In some embodiments, R group of GlcN-IdoA disaccharide of formula 96 is a C5 linear aliphatic primary amine (—(CH₂)₅NH₂).

In some embodiments, the GlcN-IdoA disaccharide is one of the HS disaccharides shown in FIG. 20. Specifically, the GlcN-IdoA disaccharide can one of the following formulas shown as compounds 12-23 in FIG. 20.

To further increase the structural diversity of the oligosaccharide, stereoisomers and their derivatives including any suitable salts (e.g. sodium salt) based on the disaccharides and tetrasaccharide building blocks can also be synthesized.

In some embodiments, the oligosaccharides synthesized herein are represented by formula 100:

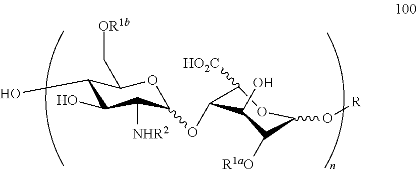

wherein n is any integer ranging from 2 to 200; R^{1a}, R^{1b} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

In some embodiments, the oligosaccharides of GlcN-IdoA disaccharide are represented by formula 101:

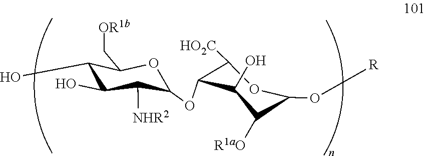

wherein n is any integer ranging from 2 to 200; R^{1a}, R^{1b} and R² are independently H, methyl, ethyl, acetyl (Ac), SO₃K, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

Disclosed herein also includes a GlcN-IdoA-GlcN-IdoA tetrasaccharide. In some embodiments, a GlcN-IdoA-GlcN-IdoA tetrasaccharide is represented by formula 101a:

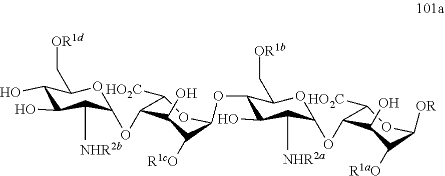

wherein R^{1a}, R^{1b}, R^{1c}, R^{1d}, R^{2a} and R^{2b} are independently H, methyl, ethyl, acetyl (Ac), S03K PO2(OCH₃)H or PO₃O₂; and R is H, a C--C12 linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a}, R^{1b}, R^{2a}, R^{2b}, R^{1c}, and/or R^{1d} is an orthogonal protecting group (e.g, hydroxyl or amine protecting group).

In some embodiments, a GlcN-IdoA-GlcN-IdoA tetrasaccharide is represented by formula 101a, wherein at least one of R^{2a} and R^{2b} is an acetyl group (Ac). In some embodiments, R^{2a} and R^{2b} are an acetyl group (Ac).

In some embodiments, an N-sulfated GlcN-IdoA-GlcN-IdoA tetrasaccharide is represented by formula 101a, wherein R^{2a} and R^{2b} are H or SO₃H with the proviso that at least one of R^{2a} and R^{2b} is SO₃H. In some embodiments, an N-sulfated GlcN-IdoA-GlcN-IdoA tetrasaccharide is represented by formula 101a, wherein R^{2a} and R^{2b} are SO₃H. In some embodiments, R group of the N-sulfated GlcN-IdoA-GlcN-IdoA tetrasaccharide of formula 101a is a C1-C12 linear aliphatic primary amine. In some embodiments, R group of the N-sulfated GlcN-IdoA-GlcN-IdoA tetrasaccharide of formula 101a is a C2-C8 linear aliphatic primary amine. In some embodiments, R group of the N-sulfated GlcN-IdoA-GlcN-IdoA tetrasaccharide of formula 101a is a C5 linear aliphatic primary amine (—(CH₂)₅NH₂).

In some embodiments, an O-sulfated GlcN-IdoA-GlcN-IdoA tetrasaccharide is represented by formula 101a, wherein R^{1a}, R^{1b}, R^{1c} and R^{1d} are H or SO₃H with the proviso that at least one of R^{1a}, R^{1b}, R^{1c} and R^{1d} is SO₃H. In some embodiments, an O-sulfated GlcN-IdoA-GlcN-IdoA tetrasaccharide is represented by formula 101a, wherein R^{1a}, R^{1b}, R^{1c} and R^{1d} are SO₃H. In some embodiments, R group of the O-sulfated GlcN-IdoA-GlcN-IdoA tetrasaccharide of formula 101a is a C1-C12 linear aliphatic primary amine. In some embodiments, R group of the N-sulfated GlcN-IdoA-GlcN-IdoA tetrasaccharide of formula 101a is a C2-C8 linear aliphatic primary amine. In some embodiments, R group of the N-sulfated GlcN-IdoA-GlcN-IdoA tetrasaccharide of formula 101a is a C5 linear aliphatic primary amine (—(CH₂)₅NH₂).

In some embodiments, an N-sulfated, O-sulfated GlcN-IdoA-GlcN-IdoA tetrasaccharide is represented by formula 101a, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated GlcN-IdoA-GlcN-IdoA tetrasaccharide is represented by formula 101a, wherein R^{2a} and R^{2b} are SO₃H. In some embodiments, the N-sulfated, O-sulfated GlcN-IdoA-GlcN-IdoA tetrasaccharide is represented by formula 101a, wherein R^{1a}, R^{1b}, R^{1c} and R^{1d} are SO₃H. In some embodiments, R group of the N-sulfated, O-sulfated GlcN-IdoA-GlcN-IdoA tetrasaccharide of formula 101a is a C1-C12 linear aliphatic primary amine. In some embodiments, R group of the N-sulfated GlcN-IdoA-GlcN-IdoA tetrasaccharide of formula 101a is a C2-C8 linear aliphatic primary amine. In some embodiments, R group of the N-sulfated GlcN-IdoA-GlcN-IdoA tetrasaccharide of formula 101a is a C5 linear aliphatic primary amine (—(CH₂)₅NH₂).

In some embodiments, the oligosaccharides of GlcN-IdoA-GlcN-IdoA tetrasaccharide are represented by formula 102:

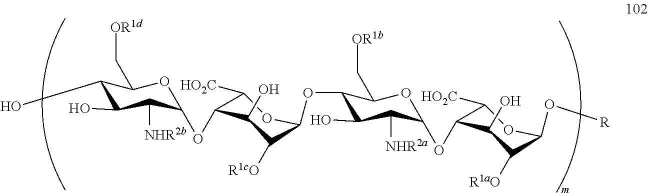

wherein m is any integer ranging from 1 to 100; R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

In some embodiments, an oligosaccharide of GlcN-IdoA-GlcN-IdoA tetrasaccharide is represented by formula 102, wherein at least one of R^{2a} and R^{2b} in 1-100 monomeric tetrasaccharide unit of formula 102 is an acetyl group (Ac). In some embodiments, R^{2a} and R^{2b} in 1-100 monomeric tetrasaccharide unit of formula 102 are an acetyl group (Ac).

In some embodiments, an N-sulfated oligosaccharide of GlcN-IdoA-GlcN-IdoA tetrasaccharide is represented by formula 102, wherein R^{2a} and R^{2b} each monomeric tetrasaccharide unit of formula 102 are each independently H or SO₃H with the proviso that at least one of R^{2a} and R^{2b} is SO₃H.

In some embodiments, an N-sulfated oligosaccharide of GlcN-IdoA-GlcN-IdoA tetrasaccharide is represented by formula 102, wherein R^{2a} and R^{2b} in 1 to 100 of monomeric units of formula 102 are SO₃H.

In some embodiments, R group of the N-sulfated oligosaccharide of GlcN-IdoA-GlcN-IdoA tetrasaccharide of formula 102 is a C1-C12 linear aliphatic primary amine. In some embodiments, R group of the oligosaccharide of GlcN-IdoA-GlcN-IdoA tetrasaccharide of formula 102 is a C2-C8 linear aliphatic primary amine. In some embodiments, R group of the oligosaccharide of GlcN-IdoA-GlcN-IdoA tetrasaccharide of formula 102 is a C5 linear aliphatic primary amine (—(CH₂)₅NH₂).

In some embodiments, an O-sulfated oligosaccharide of GlcN-IdoA-GlcN-IdoA tetrasaccharide is represented by formula 102, wherein R^{1a}, R^{1b}, R^{1c} and R^{1d} in each monomeric tetrasaccharide unit of formula 102 are each independently are H or SO₃H with the proviso that at least one of R^{1a}, R^{1b}, R^{1c} and R^{1d} is SO₃H. In some embodiments, an O-sulfated oligosaccharide of GlcN-IdoA-GlcN-IdoA tetrasaccharide is represented by formula 102, wherein R^{1a}, R^{1b}, R^{1c} and R^{1d} are SO₃H.

In some embodiments, R group of the oligomer of GlcN-IdoA-GlcN-IdoA tetrasaccharide of formula 102 is a C1-C12 linear aliphatic primary amine. In some embodiments, R group of the oligomer of GlcN-IdoA-GlcN-IdoA tetrasaccharide of formula 102 is a C2-C8 linear aliphatic primary amine. In some embodiments, R group of the oligomer of GlcN-IdoA-GlcN-IdoA tetrasaccharide of formula 102 is a C5 linear aliphatic primary amine (—(CH₂)₅NH₂).

In some embodiments, an oligomer of N-sulfated, O-sulfated GlcN-IdoA-GlcN-IdoA tetrasaccharide is represented by formula 102, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated oligomer of GlcN-IdoA-GlcN-IdoA tetrasaccharide is represented by formula 102, wherein R^{2a} and R^{2b} in 1-100 monomeric tetrasaccharide unit of formula 102 are SO₃H. In some embodiments, the N-sulfated, O-sulfated oligomer of GlcN-IdoA-GlcN-IdoA tetrasaccharide is represented by formula 102, wherein R^{1a}, R^{1b}, R^{1c} and R^{1d} in 1-100 monomeric tetrasaccharide unit of formula 102 are SO₃H. In some embodiments, R group of the N-sulfated, O-sulfated oligomer of GlcN-IdoA-GlcN-IdoA tetrasaccharide of formula 102 is a C1-C12 linear aliphatic primary amine. In some embodiments, R group of the N-sulfated oligomer of GlcN-IdoA-GlcN-IdoA tetrasaccharide of formula 102 is a C2-C8 linear aliphatic primary amine. In some embodiments, R group of the N-sulfated oligomer of GlcN-IdoA-GlcN-IdoA tetrasaccharide of formula 102 is a C5 linear aliphatic primary amine (—(CH₂)₅NH₂).

In some embodiments, the oligosaccharides of GlcN-GlcA disaccharide are represented by formula 103:

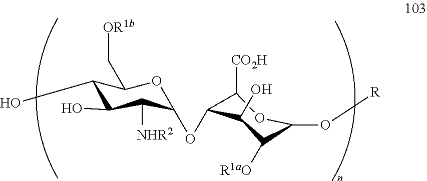

wherein n is any integer ranging from 2 to 200; R^{1a}, R^{1b} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

In some embodiments, a GlcN-GlcA-GlcN-GlcA tetrasaccharide is represented by formula 103a:

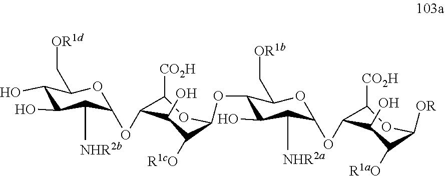

wherein R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a}, R^{1b}, R^{2a}, R^{2b}, R^{1c}, and/or R^{1d} is an orthogonal protecting group (e.g, hydroxyl or amine protecting group).

In some embodiments, a GlcN-GlcA-GlcN-GlcA tetrasaccharide is represented by formula 103a, wherein at least one of R^{2a} and R^{2b} is an acetyl group (Ac). In some embodiments, R^{2a} and R^{2b} are an acetyl group (Ac).

In some embodiments, an N-sulfated GlcN-GlcA-GlcN-GlcA tetrasaccharide is represented by formula 103a, wherein R^{2a} and R^{2b} are H or SO₃H with the proviso that at least one of R^{2a} and R^{2b} is SO₃H. In some embodiments, an N-sulfated GlcN-GlcA-GlcN-GlcA tetrasaccharide is represented by formula 103a, wherein R^{2a} and R^{2b} are SO₃H. In some embodiments, R group of the N-sulfated GlcN-GlcA-GlcN-GlcA tetrasaccharide of formula 103a is a C1-C12 linear aliphatic primary amine. In some embodiments, R group of the N-sulfated GlcN-GlcA-GlcN-GlcA tetrasaccharide of formula 103a is a C2-C8 linear aliphatic primary amine. In some embodiments, R group of the N-sulfated GlcN-GlcA-GlcN-GlcA tetrasaccharide of formula 103a is a C5 linear aliphatic primary amine (—(CH₂)₅NH₂).

In some embodiments, an O-sulfated GlcN-GlcA-GlcN-GlcA tetrasaccharide is represented by formula 103a, wherein R^{1a}, R^{1b}, R^{1c} and R^{1d} are H or SO₃H with the proviso that at least one of R^{1a}, R^{1b}, R^{1c} and R^{1d} is SO₃H. In some embodiments, an O-sulfated GlcN-GlcA-GlcN-GlcA tetrasaccharide is represented by formula 103a, wherein R^{1a}, R^{1b}, R^{1c} and R^{1d} are SO₃H. In some embodiments, R group of the O-sulfated GlcN-GlcA-GlcN-GlcA tetrasaccharide of formula 103a is a C1-C12 linear aliphatic primary amine. In some embodiments, R group of the N-sulfated GlcN-GlcA-GlcN-GlcA tetrasaccharide of formula 103a is a C2-C8 linear aliphatic primary amine. In some embodiments, R group of the N-sulfated GlcN-GlcA-GlcN-GlcA tetrasaccharide of formula 103a is a C5 linear aliphatic primary amine (—(CH₂)₅NH₂).

In some embodiments, an N-sulfated, O-sulfated GlcN-GlcA-GlcN-GlcA tetrasaccharide is represented by formula 103a, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated GlcN-GlcA-GlcN-GlcA tetrasaccharide is represented by formula 103a, wherein R^{2a} and R^{2b} are SO₃H. In some embodiments, the N-sulfated, O-sulfated GlcN-GlcA-GlcN-GlcA tetrasaccharide is represented by formula 103a, wherein R^{1a}, R^{1b}, R^{1c} and R^{1d} are SO₃H. In some embodiments, R group of the N-sulfated, O-sulfated GlcN-GlcA-GlcN-GlcA tetrasaccharide of formula 103a is a C1-C12 linear aliphatic primary amine. In some embodiments, R group of the N-sulfated GlcN-GlcA-GlcN-GlcA tetrasaccharide of formula 103a is a C2-C8 linear aliphatic primary amine. In some embodiments, R group of the N-sulfated GlcN-GlcA-GlcN-GlcA tetrasaccharide of formula 103a is a C5 linear aliphatic primary amine (—(CH₂)₅NH₂).

In some embodiments, the oligosaccharides of GlcN-GlcA-GlcN-GlcA tetrasaccharide are represented by formula 104:

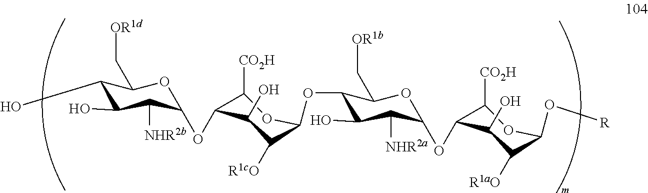

wherein m is any integer ranging from 1 to 100; R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

In some embodiments, an oligosaccharide of GlcN-GlcA-GlcN-GlcA tetrasaccharide is represented by formula 104, wherein at least one of R^{2a} and R^{2b} in 1-100 monomeric tetrasaccharide unit of formula 104 is an acetyl group (Ac). In some embodiments, R^{2a} and R^{2b} in 1-100 monomeric tetrasaccharide unit of formula 104 are an acetyl group (Ac).

In some embodiments, an N-sulfated oligosaccharide of GlcN-GlcA-GlcN-GlcA tetrasaccharide is represented by formula 104, wherein R^{2a} and R^{2b} each monomeric tetrasaccharide unit of formula 104 are each independently H or SO₃H with the proviso that at least one of R^{2a} and R^{2b} is SO₃H.

In some embodiments, an N-sulfated oligosaccharide of GlcN-GlcA-GlcN-GlcA tetrasaccharide is represented by formula 104, wherein R^{2a} and R^{2b} in 1 to 100 of monomeric units of formula 104 are SO₃H.

In some embodiments, R group of the N-sulfated oligosaccharide of GlcN-GlcA-GlcN-GlcA tetrasaccharide of formula 104 is a C1-C12 linear aliphatic primary amine. In some embodiments, R group of the oligosaccharide of GlcN-GlcA-GlcN-GlcA tetrasaccharide of formula 104 is a C2-C8 linear aliphatic primary amine. In some embodiments, R group of the oligosaccharide of GlcN-GlcA-GlcN-GlcA tetrasaccharide of formula 104 is a C5 linear aliphatic primary amine (—(CH₂)₅NH₂).

In some embodiments, an O-sulfated oligosaccharide of GlcN-GlcA-GlcN-GlcA tetrasaccharide is represented by formula 104, wherein R^{1a}, R^{1b}, R^{1c} and R^{1d} in each monomeric tetrasaccharide unit of formula 104 are each independently are H or SO₃H with the proviso that at least one of R^{1a}, R^{1b}, R^{1c} and R^{1d} is SO₃H. In some embodiments, an O-sulfated oligosaccharide of GlcN-GlcA-GlcN-GlcA tetrasaccharide is represented by formula 104, wherein R^{1a}, R^{1b}, R^{1c} and R^{1d} are SO₃H.

In some embodiments, R group of the oligomer of GlcN-GlcA-GlcN-GlcA tetrasaccharide of formula 104 is a C1-C12 linear aliphatic primary amine. In some embodiments, R group of the oligomer of GlcN-GlcA-GlcN-GlcA tetrasaccharide of formula 104 is a C2-C8 linear aliphatic primary amine. In some embodiments, R group of the oligomer of GlcN-GlcA-GlcN-GlcA tetrasaccharide of formula 104 is a C5 linear aliphatic primary amine (—(CH₂)₅NH₂).

In some embodiments, an oligomer of N-sulfated, O-sulfated GlcN-GlcA-GlcN-GlcA tetrasaccharide is represented by formula 104, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated oligomer of GlcN-GlcA-GlcN-GlcA tetrasaccharide is represented by formula 104, wherein R^{2a} and R^{2b} in 1-100 monomeric tetrasaccharide unit of formula 102 are SO₃H. In some embodiments, the N-sulfated, O-sulfated oligomer of GlcN-GlcA-GlcN-GlcA tetrasaccharide is represented by formula 104, wherein R^{1a}, R^{1b}, R^{1c} and R^{1d} in 1-100 monomeric tetrasaccharide unit of formula 104 are SO₃H. In some embodiments, R group of the N-sulfated, O-sulfated oligomer of GlcN-GlcA-GlcN-GlcA tetrasaccharide of formula 104 is a C1-C12 linear aliphatic primary amine. In some embodiments, R group of the N-sulfated oligomer of GlcN-GlcA-GlcN-GlcA tetrasaccharide of formula 104 is a C2-C8 linear aliphatic primary amine. In some embodiments, R group of the N-sulfated oligomer of GlcN-GlcA-GlcN-GlcA tetrasaccharide of formula 104 is a C5 linear aliphatic primary amine (—(CH₂)₅NH₂).

In some embodiments, a GlcN-GlcA-GlcN-IdoA tetrasaccharide represented by formula 100a:

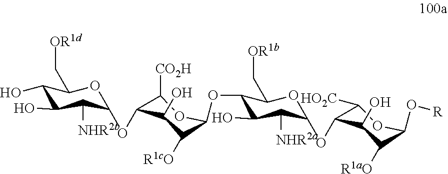

wherein R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a}, R^{1b}, R^{2a}, R^{2b}, R^{1c}, and/or R^{1d} is a protecting group (e.g, hydroxyl or amine protecting group).

In some embodiments, a GlcN-GlcA-GlcN-IdoA tetrasaccharide is represented by formula 100a, wherein at least one of R^{2a} and R^{2b} is an acetyl group (Ac). In some embodiments, R^{2a} and R^{2b} are an acetyl group (Ac).

In some embodiments, an N-sulfated GlcN-GlcA-GlcN-IdoA tetrasaccharide is represented by formula 100a, wherein R^{2a} and R^{2b} are H or SO₃H with the proviso that at least one of R^{2a} and R^{2b} is SO₃H. In some embodiments, an N-sulfated GlcN-GlcA-GlcN-IdoA tetrasaccharide is represented by formula 100a, wherein R^{2a} and R^{2b} are SO₃H. In some embodiments, R group of the N-sulfated GlcN-GlcA-GlcN-IdoA tetrasaccharide of formula 100a is a C1-C12 linear aliphatic primary amine. In some embodiments, R group of the N-sulfated GlcN-GlcA-GlcN-IdoA tetrasaccharide of formula 100a is a C2-C8 linear aliphatic primary amine. In some embodiments, R group of the N-sulfated GlcN-GlcA-GlcN-IdoA tetrasaccharide of formula 100a is a C5 linear aliphatic primary amine (—(CH₂)₅NH₂).

In some embodiments, an O-sulfated GlcN-GlcA-GlcN-IdoA tetrasaccharide is represented by formula 100a, wherein R^{1a}, R^{1b}, R^{1c} and R^{1d} are H or SO₃H with the proviso that at least one of R^{1a}, R^{1b}, R^{1c} and R^{1d} is SO₃H. In some embodiments, an O-sulfated GlcN-GlcA-GlcN-IdoA tetrasaccharide is represented by formula 100a, wherein R^{1a}, R^{1b}, R^{1c} and R^{1d} are SO₃H. In some embodiments, R group of the O-sulfated GlcN-GlcA-GlcN-IdoA tetrasaccharide of formula 100a is a C1-C12 linear aliphatic primary amine. In some embodiments, R group of the N-sulfated GlcN-GlcA-GlcN-IdoA tetrasaccharide of formula 100a is a C2-C8 linear aliphatic primary amine. In some embodiments, R group of the N-sulfated GlcN-GlcA-GlcN-IdoA tetrasaccharide of formula 100a is a C5 linear aliphatic primary amine (—(CH₂)₅NH₂).

In some embodiments, an N-sulfated, O-sulfated GlcN-GlcA-GlcN-IdoA tetrasaccharide is represented by formula 100a, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated GlcN-GlcA-GlcN-IdoA tetrasaccharide is represented by formula 100a, wherein R^{2a} and R^{2b} are SO₃H. In some embodiments, the N-sulfated, O-sulfated GlcN-GlcA-GlcN-IdoA tetrasaccharide is represented by formula 100a, wherein R^{1a}, R^{1b}, R^{1c} and R^{1d} are SO₃H. In some embodiments, R group of the N-sulfated, O-sulfated GlcN-GlcA-GlcN-IdoA tetrasaccharide of formula 100a is a C1-C12 linear aliphatic primary amine. In some embodiments, R group of the N-sulfated GlcN-GlcA-GlcN-IdoA tetrasaccharide of formula 100a is a C2-C8 linear aliphatic primary amine. In some embodiments, R group of the N-sulfated GlcN-GlcA-GlcN-IdoA tetrasaccharide of formula 100a is a C5 linear aliphatic primary amine (—(CH₂)₅NH₂).

In some embodiments, the oligosaccharides of GlcN-GlcA-GlcN-IdoA tetrasaccharide are represented by formula 105:

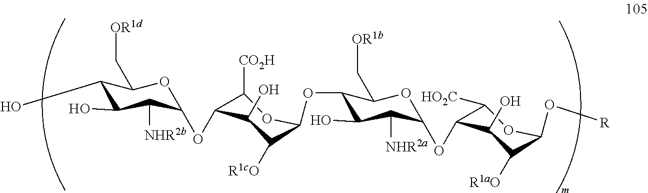

wherein m is any integer ranging from 1 to 100; R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

In some embodiments, an oligosaccharide of GlcN-GlcA-GlcN-IdoA tetrasaccharide is represented by formula 105, wherein at least one of R^{2a} and R^{2b} in 1-100 monomeric tetrasaccharide unit of formula 105 is an acetyl group (Ac). In some embodiments, R^{2a} and R^{2b} in 1-100 monomeric tetrasaccharide unit of formula 105 are an acetyl group (Ac).

In some embodiments, an N-sulfated oligosaccharide of GlcN-GlcA-GlcN-IdoA tetrasaccharide is represented by formula 105, wherein R^{2a} and R^{2b} each monomeric tetrasaccharide unit of formula 105 are each independently H or SO₃H with the proviso that at least one of R^{2a} and R^{2b} is SO₃H.

In some embodiments, an N-sulfated oligosaccharide of GlcN-GlcA-GlcN-IdoA tetrasaccharide is represented by formula 105, wherein R^{2a} and R^{2b} in 1 to 100 of monomeric units of formula 105 are SO₃H.

In some embodiments, R group of the N-sulfated oligosaccharide of GlcN-GlcA-GlcN-IdoA tetrasaccharide of formula 105 is a C1-C12 linear aliphatic primary amine. In some embodiments, R group of the oligosaccharide of GlcN-GlcA-GlcN-IdoA tetrasaccharide of formula 105 is a C2-C8 linear aliphatic primary amine. In some embodiments, R group of the oligosaccharide of GlcN-GlcA-GlcN-IdoA tetrasaccharide of formula 105 is a C5 linear aliphatic primary amine (—(CH₂)₅NH₂).

In some embodiments, an O-sulfated oligosaccharide of GlcN-GlcA-GlcN-IdoA tetrasaccharide is represented by formula 105, wherein R^{1a}, R^{1b}, R^{1c} and R^{1d} in each monomeric tetrasaccharide unit of formula 105 are each independently are H or SO₃H with the proviso that at least one of R^{1a}, R^{1b}, R^{1c} and R^{1d} is SO₃H. In some embodiments, an O-sulfated oligosaccharide of GlcN-GlcA-GlcN-IdoA tetrasaccharide is represented by formula 105, wherein R^{1a}, R^{1b}, R^{1c} and R^{1d} are SO₃H.

In some embodiments, R group of the oligomer of GlcN-GlcA-GlcN-IdoA tetrasaccharide of formula 105 is a C1-C12 linear aliphatic primary amine. In some embodiments, R group of the oligomer of GlcN-GlcA-GlcN-IdoA tetrasaccharide of formula 105 is a C2-C8 linear aliphatic primary amine. In some embodiments, R group of the oligomer of GlcN-GlcA-GlcN-IdoA tetrasaccharide of formula 105 is a C5 linear aliphatic primary amine (—(CH₂)₅NH₂).

In some embodiments, an oligomer of N-sulfated, O-sulfated GlcN-GlcA-GlcN-IdoA tetrasaccharide is represented by formula 105, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated oligomer of GlcN-GlcA-GlcN-IdoA tetrasaccharide is represented by formula 105, wherein R^{2a} and R^{2b} in 1-100 monomeric tetrasaccharide unit of formula 105 are SO₃H. In some embodiments, the N-sulfated, O-sulfated oligomer of GlcN-GlcA-GlcN-IdoA tetrasaccharide is represented by formula 105, wherein R^{1a}, R^{1b}, R^{1c} and R^{1d} in 1-100 monomeric tetrasaccharide unit of formula 105 are SO₃H. In some embodiments, R group of the N-sulfated, O-sulfated oligomer of GlcN-GlcA-GlcN-IdoA tetrasaccharide of formula 105 is a C1-C12 linear aliphatic primary amine. In some embodiments, R group of the N-sulfated oligomer of GlcN-GlcA-GlcN-IdoA tetrasaccharide of formula 105 is a C2-C8 linear aliphatic primary amine. In some embodiments, R group of the N-sulfated oligomer of GlcN-GlcA-GlcN-IdoA tetrasaccharide of formula 105 is a C5 linear aliphatic primary amine (—(CH₂)₅NH₂).

In some embodiments, a GlcN-IdoA-GlcN-GlcA tetrasaccharide is represented by formula 100b:

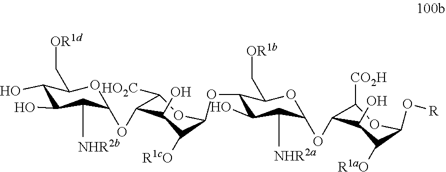

wherein R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine. In some embodiments, R is a fluorous tag. In some embodiments, R^{1a}, R^{1b}, R^{2a}, R^{2b}, R^{1c}, and/or R^{1d} is a protecting group (e.g, hydroxyl or amine protecting group).

In some embodiments, a GlcN-IdoA-GlcN-GlcA tetrasaccharide is represented by formula 100b, wherein at least one of R^{2a} and R^{2b} is an acetyl group (Ac). In some embodiments, R^{2a} and R^{2b} are an acetyl group (Ac).

In some embodiments, an N-sulfated GlcN-IdoA-GlcN-GlcA tetrasaccharide is represented by formula 100b, wherein R^{2a} and R^{2b} are H or SO₃H with the proviso that at least one of R^{2a} and R^{2b} is SO₃H. In some embodiments, an N-sulfated GlcN-IdoA-GlcN-GlcA tetrasaccharide is represented by formula 100b, wherein R^{2a} and R^{2b} are SO₃H. In some embodiments, R group of the N-sulfated GlcN-IdoA-GlcN-GlcA tetrasaccharide of formula 100b is a C1-C12 linear aliphatic primary amine. In some embodiments, R group of the N-sulfated GlcN-IdoA-GlcN-GlcA tetrasaccharide of formula 100b is a C2-C8 linear aliphatic primary amine. In some embodiments, R group of the N-sulfated GlcN-IdoA-GlcN-GlcA tetrasaccharide of formula 100b is a C5 linear aliphatic primary amine (—(CH₂)₅NH₂).

In some embodiments, an O-sulfated GlcN-IdoA-GlcN-GlcA tetrasaccharide is represented by formula 100b, wherein R^{1a}, R^{1b}, R^{1c} and R^{1d} are H or SO₃H with the proviso that at least one of R^{1a}, R^{1b}, R^{1c} and R^{1d} is SO₃H. In some embodiments, an O-sulfated GlcN-IdoA-GlcN-GlcA tetrasaccharide is represented by formula 100b, wherein R^{1a}, R^{1b}, R^{1c} and R^{1d} are SO₃H. In some embodiments, R group of the O-sulfated GlcN-IdoA-GlcN-GlcA tetrasaccharide of formula 100b is a C1-C12 linear aliphatic primary amine. In some embodiments, R group of the N-sulfated GlcN-IdoA-GlcN-GlcA tetrasaccharide of formula 100b is a C2-C8 linear aliphatic primary amine. In some embodiments, R group of the N-sulfated GlcN-IdoA-GlcN-GlcA tetrasaccharide of formula 100b is a C5 linear aliphatic primary amine (—(CH₂)₅NH₂).

In some embodiments, an N-sulfated, O-sulfated GlcN-IdoA-GlcN-GlcA tetrasaccharide is represented by formula 100b, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated GlcN-IdoA-GlcN-GlcA tetrasaccharide is represented by formula 100b, wherein R^{2a} and R^{2b} are SO₃H. In some embodiments, the N-sulfated, O-sulfated GlcN-IdoA-GlcN-GlcA tetrasaccharide is represented by formula 100b, wherein R^{1a}, R^{1b}, R^{1c} and R^{1d} are SO₃H. In some embodiments, R group of the N-sulfated, O-sulfated GlcN-IdoA-GlcN-GlcA tetrasaccharide of formula 100b is a C1-C12 linear aliphatic primary amine. In some embodiments, R group of the N-sulfated GlcN-IdoA-GlcN-GlcA tetrasaccharide of formula 100b is a C2-C8 linear aliphatic primary amine. In some embodiments, R group of the N-sulfated GlcN-IdoA-GlcN-GlcA tetrasaccharide of formula 100b is a C5 linear aliphatic primary amine (—(CH₂)₅NH₂).

In some embodiments, the oligosaccharides of GlcN-IdoA-GlcN-GlcA tetrasaccharide are represented by formula 106:

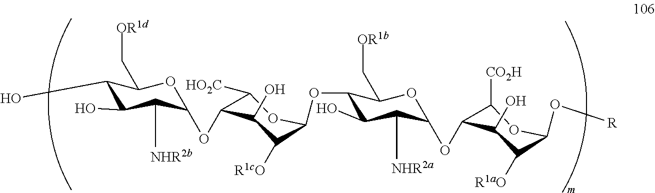

wherein m is any integer ranging from 1 to 100; R^{1a}, R^{1b}, R^{1c}, R^{1d}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine. Also disclosed herein include HS oligosaccharides (e.g., HS oligosaccharide with n=2-100) attached in multivalent form to a polymeric scaffold such as a ROMP polymer, peptide backbone and/or aptamer.

In some embodiments, an oligosaccharide of GlcN-IdoA-GlcN-GlcA tetrasaccharide is represented by formula 106, wherein at least one of R^{2a} and R^{2b} in 1-100 monomeric tetrasaccharide unit of formula 106 is an acetyl group (Ac). In some embodiments, R^{2a} and R^{2b} in 1-100 monomeric tetrasaccharide unit of formula 106 are an acetyl group (Ac).

In some embodiments, an N-sulfated oligosaccharide of GlcN-IdoA-GlcN-GlcA tetrasaccharide is represented by formula 106, wherein R^{2a} and R^{2b} each monomeric tetrasaccharide unit of formula 106 are each independently H or SO₃H with the proviso that at least one of R^{2a} and R^{2b} is SO₃H.

In some embodiments, an N-sulfated oligosaccharide of GlcN-IdoA-GlcN-GlcA tetrasaccharide is represented by formula 106, wherein R^{2a} and R^{2b} in 1 to 100 of monomeric units of formula 106 are SO₃H.

In some embodiments, R group of the N-sulfated oligosaccharide of GlcN-IdoA-GlcN-GlcA tetrasaccharide of formula 106 is a C1-C12 linear aliphatic primary amine. In some embodiments, R group of the oligosaccharide of GlcN-IdoA-GlcN-GlcA tetrasaccharide of formula 106 is a C2-C8 linear aliphatic primary amine. In some embodiments, R group of the oligosaccharide of GlcN-IdoA-GlcN-GlcA tetrasaccharide of formula 106 is a C5 linear aliphatic primary amine (—(CH₂)₅NH₂).

In some embodiments, an O-sulfated oligosaccharide of GlcN-IdoA-GlcN-GlcA tetrasaccharide is represented by formula 106, wherein R^{1a}, R^{1b}, R^{1c} and R^{1d} in each monomeric tetrasaccharide unit of formula 106 are each independently are H or SO₃H with the proviso that at least one of R^{1a}, R^{1b}, R^{1c} and R^{1d} is SO₃H. In some embodiments, an O-sulfated oligosaccharide of GlcN-IdoA-GlcN-GlcA tetrasaccharide is represented by formula 106, wherein R^{1a}, R^{1b}, R^{1c} and R^{1d} are SO₃H.

In some embodiments, R group of the oligomer of GlcN-IdoA-GlcN-GlcA tetrasaccharide of formula 106 is a C1-C12 linear aliphatic primary amine. In some embodiments, R group of the oligomer of GlcN-IdoA-GlcN-GlcA tetrasaccharide of formula 106 is a C2-C8 linear aliphatic primary amine. In some embodiments, R group of the oligomer of GlcN-IdoA-GlcN-GlcA tetrasaccharide of formula 106 is a C5 linear aliphatic primary amine (—(CH₂)₅NH₂).

In some embodiments, an oligomer of N-sulfated, O-sulfated GlcN-IdoA-GlcN-GlcA tetrasaccharide is represented by formula 106, wherein R^{2a} and R^{2b} are H or SO₃H with the proviso that at least one of R^{2a} and R^{2b} is SO₃H, and wherein R^{1a}, R^{1b}, R^{1c} and R^{1d} are H or SO₃H with the proviso that at least one of R^{1a}, R^{1b}, R^{1c} and R^{1d} is SO₃H. In some embodiments, the N-sulfated, O-sulfated oligomer of GlcN-GlcA-GlcN-IdoA tetrasaccharide is represented by formula 106, wherein R^{2a} and R^{2b} in 1-100 monomeric tetrasaccharide unit of formula 106 are SO₃H. In some embodiments, the N-sulfated, O-sulfated oligomer of GlcN-IdoA-GlcN-GlcA tetrasaccharide is represented by formula 106, wherein R^{1a}, R^{1b}, R^{1c} and R^{1d} in 1-100 monomeric tetrasaccharide unit of formula 106 are SO₃H. In some embodiments, R group of the N-sulfated, O-sulfated oligomer of GlcN-IdoA-GlcN-GlcA tetrasaccharide of formula 106 is a C1-C12 linear aliphatic primary amine. In some embodiments, R group of the N-sulfated oligomer of GlcN-IdoA-GlcN-GlcA tetrasaccharide of formula 106 is a C2-C8 linear aliphatic primary amine. In some embodiments, R group of the N-sulfated oligomer of GlcN-IdoA-GlcN-GlcA tetrasaccharide of formula 106 is a C5 linear aliphatic primary amine (—(CH₂)₅NH₂).

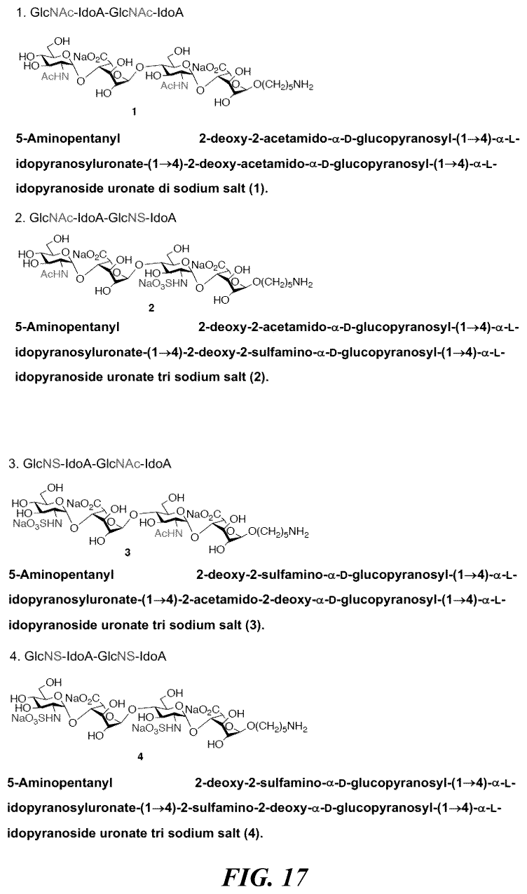

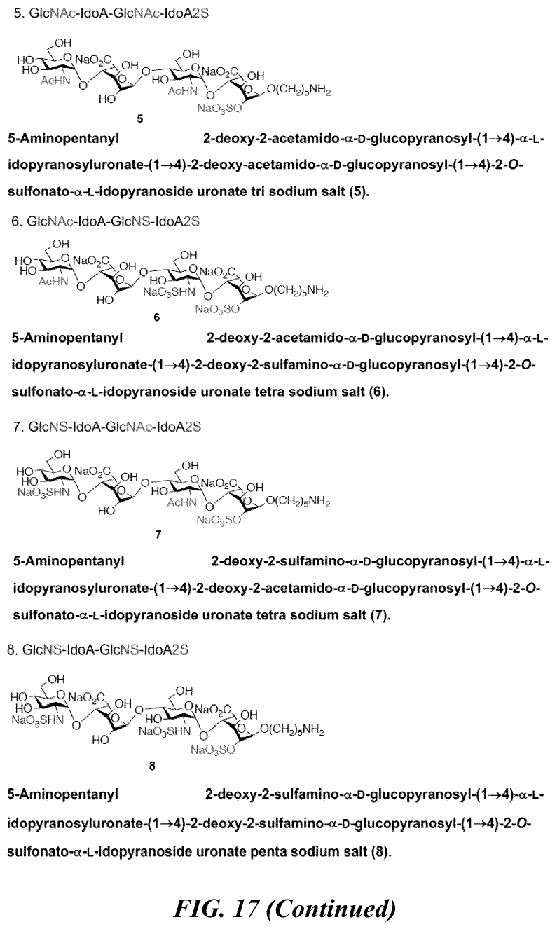

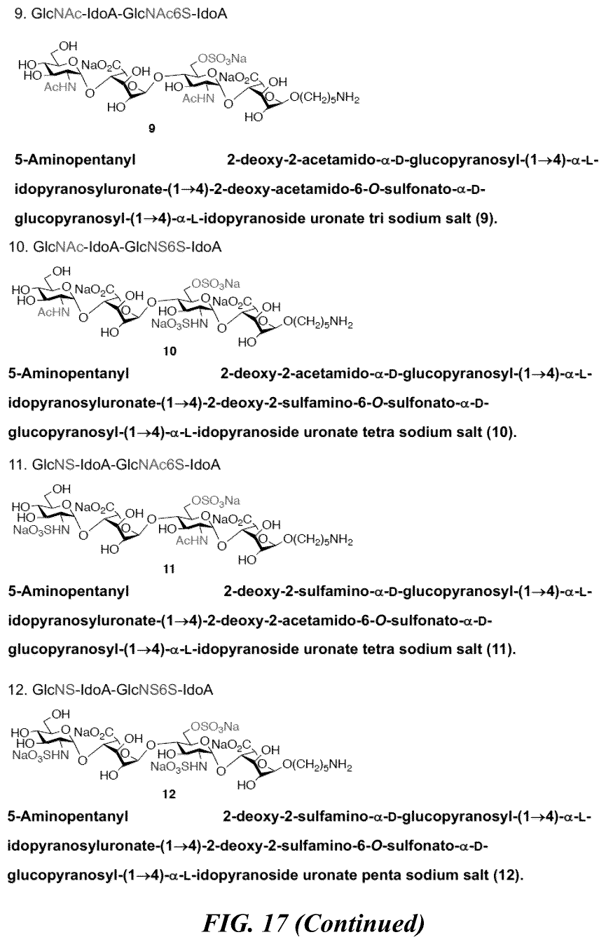

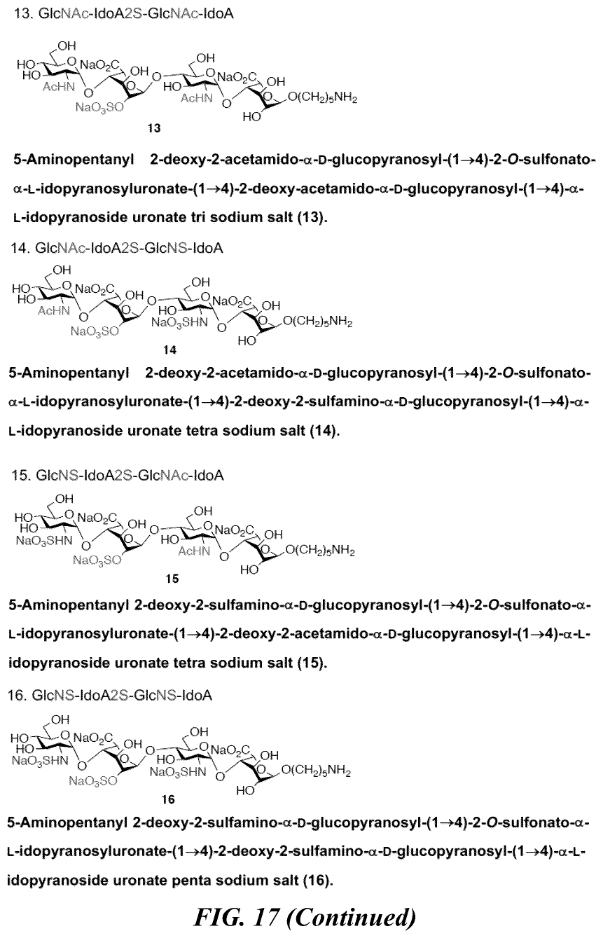

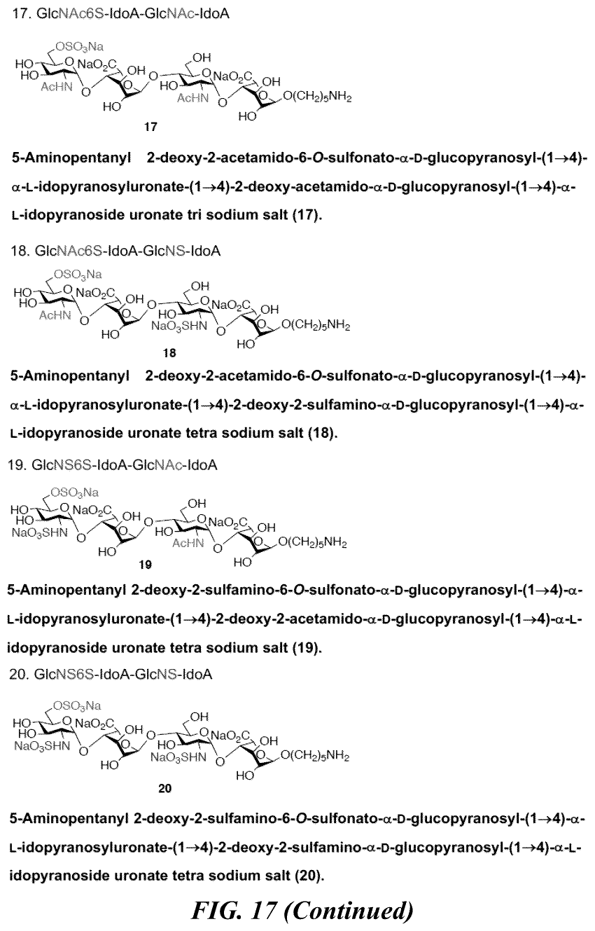

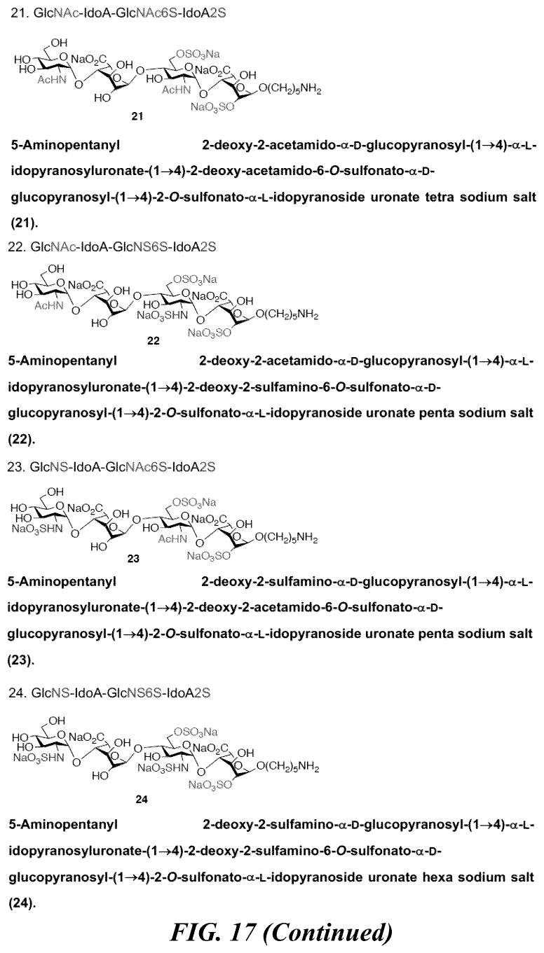

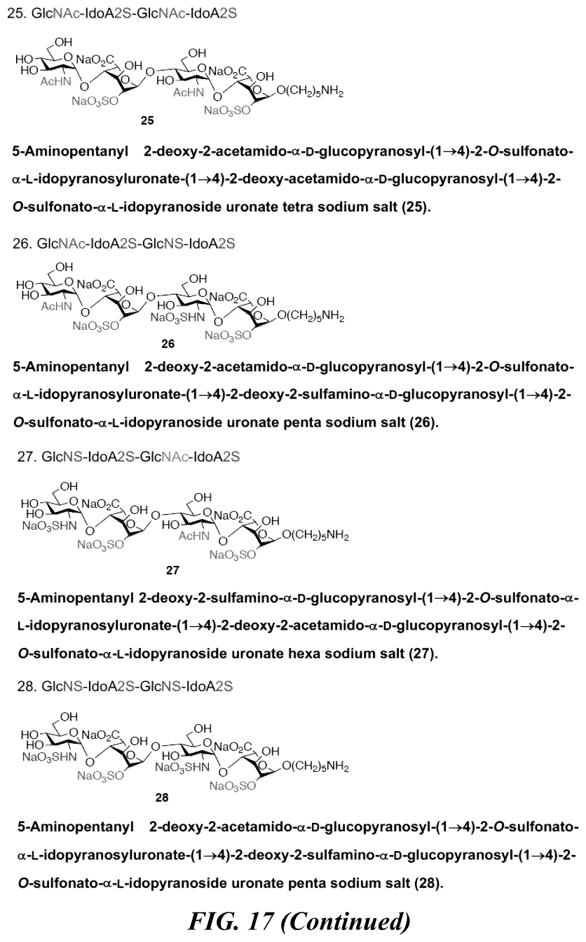

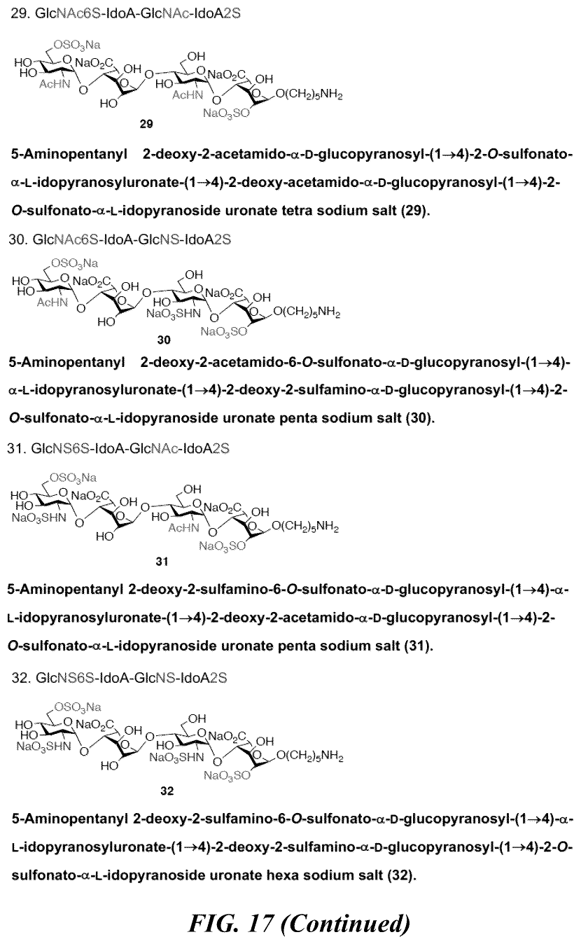

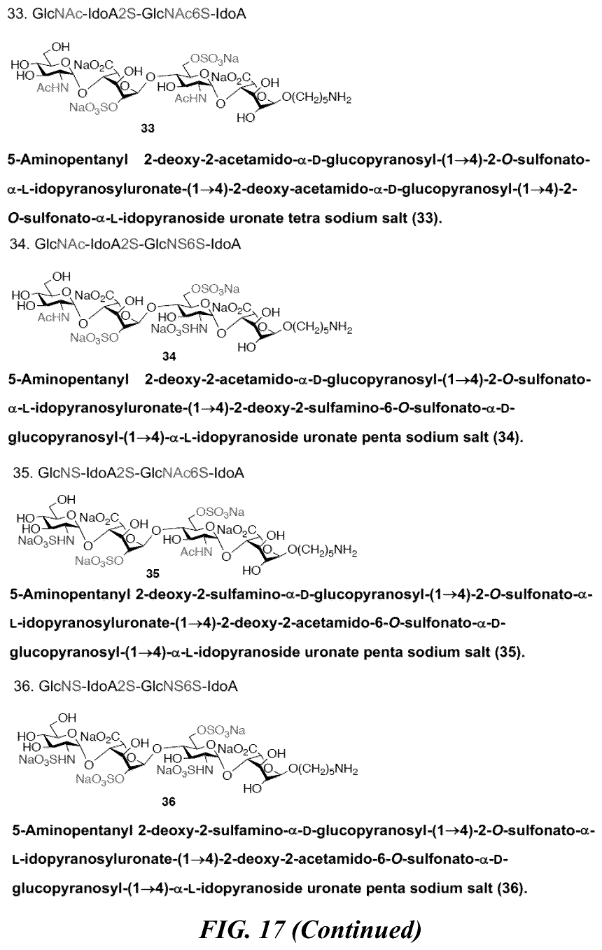

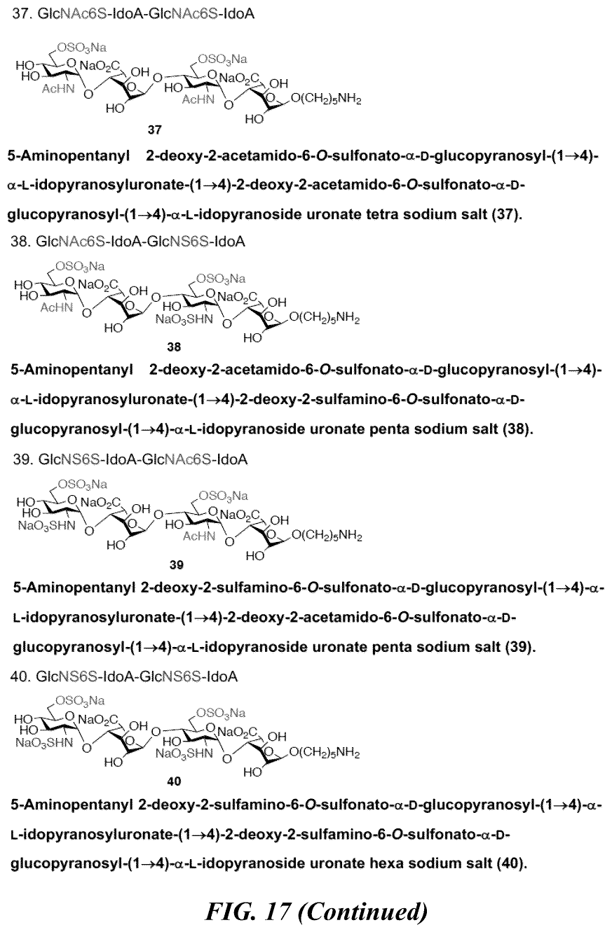

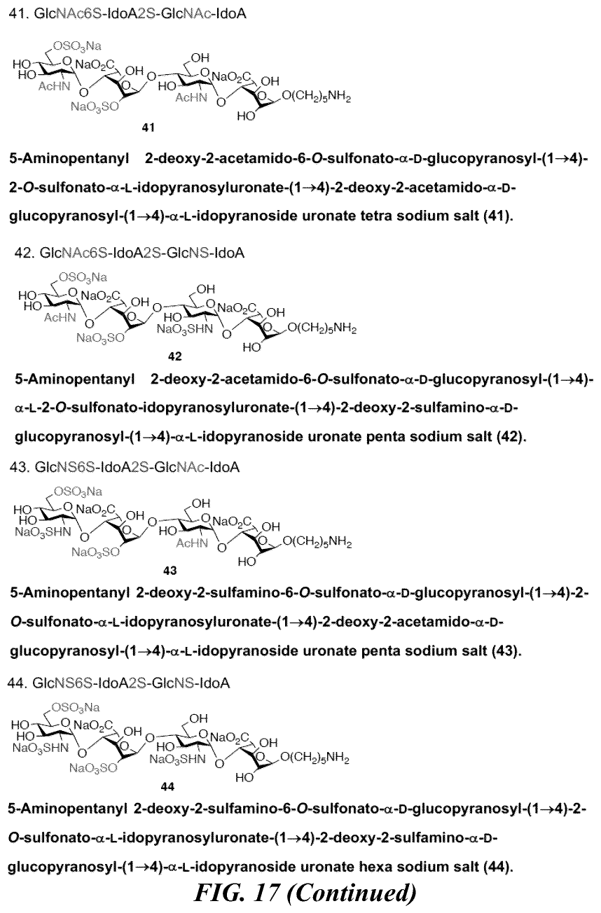

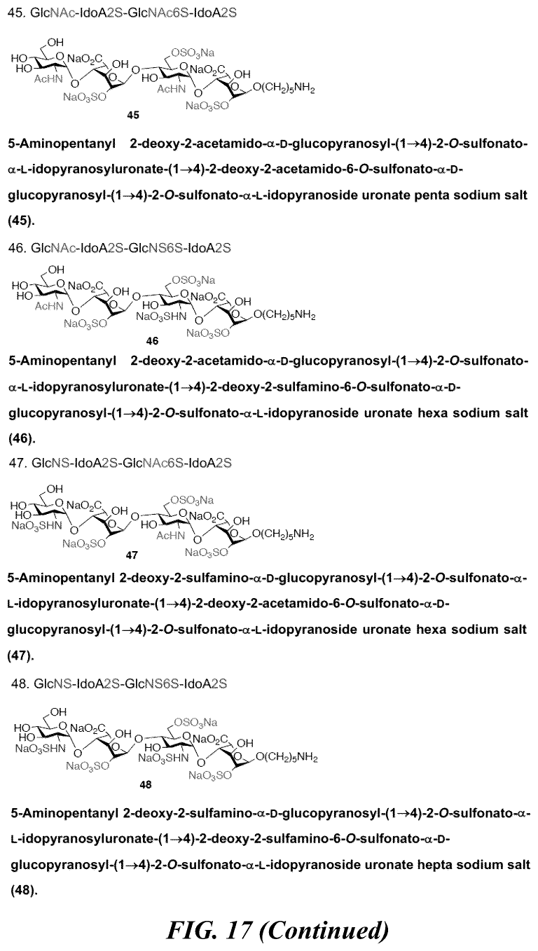

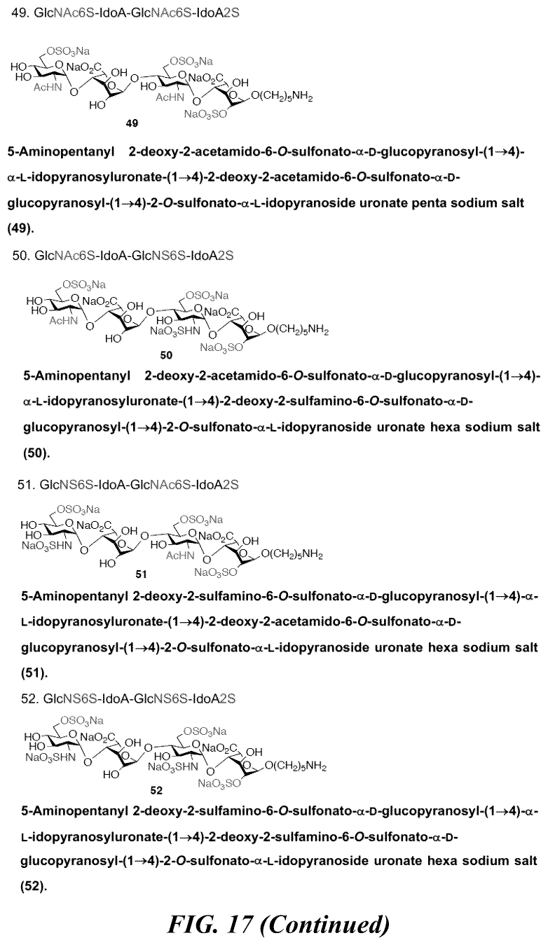

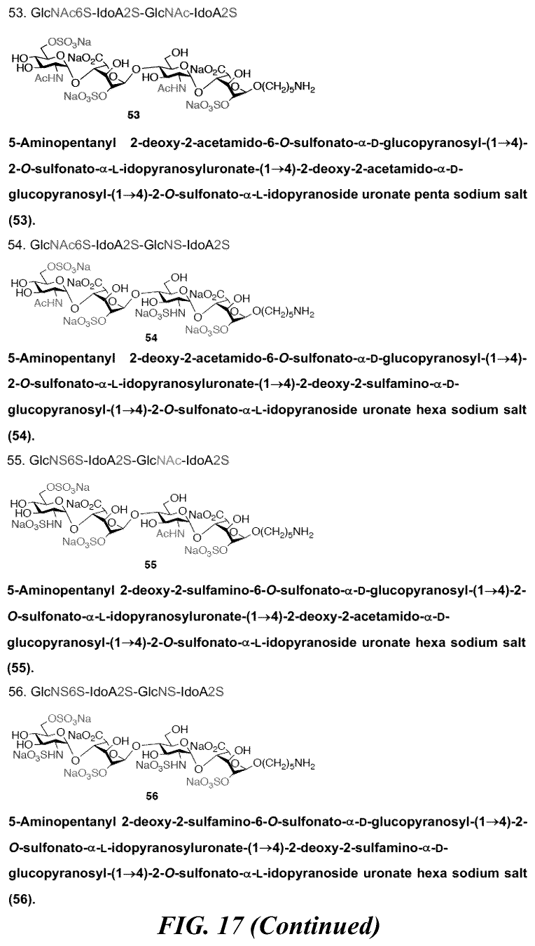

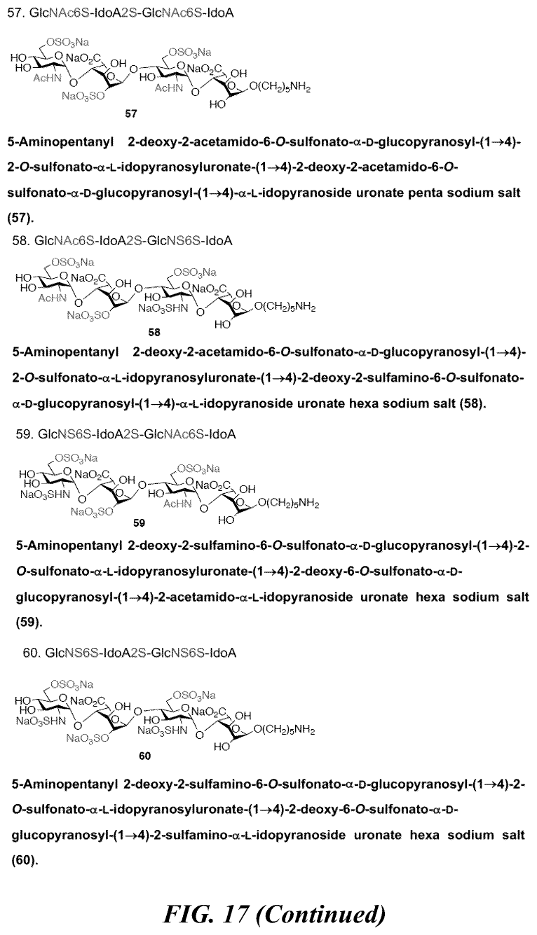

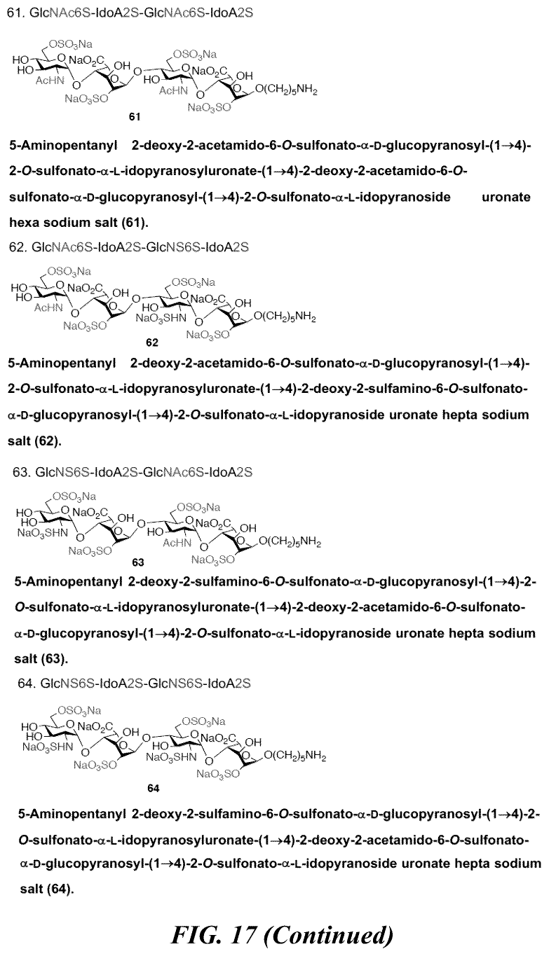

In some embodiments, the HS tetrasaccharide is one of the 64 HS tetrasaccharides illustrated in FIG. 1, panel a. Specifically, the HS tetrasaccharide can be GlcNAc-IdoA-GlcNAc-IdoA-R (1), GlcNAc-IdoA-GlcNS-IdoA-R (2), GlcNS-IdoA-GlcNAc-IdoA-R (3), GlcNS-IdoA-GlcNS-IdoA-R (4), GlcNAc-IdoA-GlcNAc-IdoA2S-R (5), GlcNAc-IdoA-GlcNS-IdoA2S-R (6), GlcNS-IdoA-GlcNAc-IdoA2S-R (7), GlcNS-IdoA-GlcNS-IdoA2S-R (8), GlcNAc-IdoA-GlcNAc6S-IdoA-R (9), GlcNAc-IdoA-GlcNS6S-IdoA-R (10), GlcNS-IdoA-GlcNAc6S-IdoA-R (11), GlcNS-IdoA-GlcNS6S-IdoA-R (12), GlcNAc-IdoA2S-GlcNAc-IdoA-R (13), GlcNAc-IdoA2S-GlcNS-IdoA-R (14), GlcNS-IdoA2S-GlcNAc-IdoA-R (15), GlcNS-IdoA2S-GlcNS-IdoA-R (16), GlcNAc6S-IdoA-GlcNAc-IdoA-R (17), GlcNAc6S-IdoA-GlcNS-IdoA-R (18), GlcNS6S-IdoA-GlcNAc-IdoA-R (19), GlcNS6S-IdoA-GlcNS-IdoA-R (20), GlcNAc-IdoA-GlcNAc6S-IdoA2S-R (21), GlcNAc-IdoA-GlcNS6S-IdoA2S-R (22), GlcNS-IdoA-GlcNAc6S-IdoA2S-R (23), GlcNS-IdoA-GlcNS6S-IdoA2S-R (24), GlcNAc-IdoA2S-GlcNAc-IdoA2S-R (25), GlcNAc-IdoA2S-GlcNS-IdoA2S-R (26), GlcNS-IdoA2S-GlcNAc-IdoA2S-R (27), GlcNS-IdoA2S-GlcNS-IdoA2S-R (28), GlcNAc6S-IdoA-GlcNAc-IdoA2S-R (29), GlcNAc6S-IdoA-GlcNS-IdoA2S-R (30), GlcNS6S-IdoA-GlcNAc-IdoA2S-R (31), GlcNS6S-IdoA-GlcNS-IdoA2S-R (32), GlcNAc-IdoA2S-GlcNAc6S-IdoA-R (33), GlcNAc-IdoA2S-GlcNS6S-IdoA-R (34), GlcNS-IdoA2S-GlcNAc6S-IdoA-R (35), GlcNS-IdoA2S-GlcNS6S-IdoA-R (36), GlcNAc6S-IdoA-GlcNAc6S-IdoA-R (37), GlcNAc6S-IdoA-GlcNS6S-IdoA-R (38), GlcNS6S-IdoA-GlcNAc6S-IdoA-R (39), GlcNS6S-IdoA-GlcNS6S-IdoA-R (40), GlcNAc6S-IdoA2S-GlcNAc-IdoA-R (41), GlcNAc6S-IdoA2S-GlcNS-IdoA-R (42), GlcNS6S-IdoA2S-GlcNAc-IdoA-R (43), GlcNS6S-IdoA2S-GlcNS-IdoA-R (44), GlcNAc-IdoA2S-GlcNAc6S-IdoA2S-R (45), GlcNAc-IdoA2S-GlcNS6S-IdoA2S-R (46), GlcNS-IdoA2S-GlcNAc6S-IdoA2S-R (47), GlcNS-IdoA2S-GlcNS6S-IdoA2S-R (48), GlcNAc6S-IdoA-GlcNAc6S-IdoA2S-R (49), GlcNAc6S-IdoA-GlcNS6S-IdoA2S-R (50), GlcNS6S-IdoA-GlcNAc6S-IdoA2S-R (51), GlcNS6S-IdoA-GlcNS6S-IdoA2S-R (52), GlcNAc6S-IdoA2S-GlcNAc-IdoA2S-R (53), GlcNAc6S-IdoA2S-GlcNS-IdoA2S-R (54), GlcNS6S-IdoA2S-GlcNAc-IdoA2S-R (55), GlcNS6S-IdoA2S-GlcNS-IdoA2S-R (56), GlcNAc6S-IdoA2S-GlcNAc6S-IdoA-R (57), GlcNAc6S-IdoA2S-GlcNS6S-IdoA-R (58), GlcNS6S-IdoA2S-GlcNAc6S-IdoA-R (59), GlcNS6S-IdoA2S-GlcNS6S-IdoA-R (60), GlcNAc6S-IdoA2S-GlcNAc6S-IdoA2S-R (61), GlcNAc6S-IdoA2S-GlcNS6S-IdoA2S-R (62), GlcNS6S-IdoA2S-GlcNAc6S-IdoA2S-R (63), and GlcNS6S-IdoA2S-GlcNS6S-IdoA2S-R (64), wherein IdoA is L-iduronic acid, GlcN is d-glucosamine, GlcNAc is N-acetyl-d-glucosamine, NS refers to N-sulfate modification, 2S refers to 2-O-sulfate modification, and 6S refers to 6-O-sulfate modification. The HS tetrasaccharide can have one of the formulas illustrated in FIG. 17.

The chemical synthesis described herein for making the HS disaccharide, tetrasaccharide, and oligosaccharide compounds uses orthogonal protection strategies. Orthogonal protecting groups are temporary protecting groups that are complementary to each other, such that each protecting group is independently removable. Orthogonal protecting groups can be cleaved under different reaction conditions without affecting the other functions present. Orthogonal protecting group strategies and conditions are reviewed in the textbooks, Greene and Wicks, Protecting Groups in Organic Synthesis, Third Edition, Wiley, New York, 1999, and Kocienski, Protecting Groups, Third Edition, Thieme, New York, 2004. A compound of the invention, such as a tetrasaccharide building block, that is “orthogonally protected” contains six different protecting groups that are orthogonal to each other, i.e., that can be removed independently without affecting the other orthogonal protecting group or any permanent protecting groups that are present on the compound. Orthogonal protecting groups used in the present invention are preferably hydroxyl protecting groups and/or amine protecting groups. The use of orthogonal protecting groups allows for controlled sulfation at selected sites, because selected sites can be deprotected and sulfated without disturbing the protecting groups at other sites which are removed at a later point in the oligosaccharide synthesis. Orthogonal cleavage in the context of the present invention is selective cleavage of a temporary hydroxy or amino protecting group from a saccharide, in which the cleavage conditions do not compromise the stability of the other protecting or functional groups on the molecule. Cleavages of protecting groups that are orthogonal to each other can be effected in any order of priority.

The orthogonally protected tetrasaccharide building block exemplified in FIG. 1, panel b and FIG. 20 includes one or more preferred protecting groups utilized in the methods and compounds described herein, but many other protecting groups can be used.

Exemplary hydroxyl protecting groups include, but are not limited to, esters such as Acetyl (Ac), Benzoyl (Bz), Chloroacetyl (ClAc), Pivaloyl (Piv), Levulinoyl (Lev), Pivaloyl levulinoyl (PivLev), Pivaloyl acetyl (PivAc), Pivaloyl benzoyl (PivBz), Difluorobenzoyl (dfBz), Bromoacetyl (BrAc); alkoxyalkyl ethers such as Methoxymethyl (MOM), (2-Methoxyethoxy) methyl (MEM) Benzyloxymethyl (BOM), p-Methyl benzyloxymethyl (pMBOM), Trimethylsilylethoxymethyl (SEM); carbonates such as 9-Fluorenylmethyl carbonyl (Fmoc), [2,2,2-Trichloroethoxycarbonyl](Troc), Allyloxycarbonyl (Alloc); ethers such as Naphthylmethyl (Nap) (which includes both 2-naphthylmethyl, 2-methyl naphthyl, NAP, and 1-naphthylmethyl, 1-NAP, and Nap ethers; Nap can be used as either a temporary or permanent protecting group), p-Methoxybenzyl (pMB), Trityl (Tr), Tetrahydro-2-pyranyl (THP), Methoxytrityl (MTr), Dimethoxytrityl (DMTr), Allyl (All); and Tosylates such as p-toluene sulfonyl (Tos), methanesulfonyl (Ms), tert-butyldiphenylsilyl (TBDPS).

Exemplary amine protecting groups include, but are not limited to, Azide (N₃), Phthalimido (Phthal), Tetrachlorophthaloyl (TCP), N-dithiasuccinyl (Dts) (this protecting group could be cleaved orthogonally in presence of an azide using propane-1,3-dithiol (PDT) and DIPEA), N—R₂, R=Acetyl or other permanent or temporary protecting group, [2,2,2-Trichloroethoxycarbonyl](Troc), Acetyl (Ac), Levulinoyl (Lev), Pivaloyl acetyl chloride (PivAc), Pivaloyl levulinoyl chloride (PivLev), Tosyl (Tos), Nosyl (Nos), Allyloxycarbonyl (Alloc), Trichloroacetyl (TCA), Trifluoroacetyl (TFA), Trityl (Tr), Benzylideneamine, Tert-butyloxycarbonyl (Boc), Benzyloxy carbonyl (Cbz), Oxazolidine, Dimethylmaleoyl (DMM), Thiodiglycolyl (TDG), Diphenylmaleoyl (DPM), Diglycolyl (DG), and Dimethylglutamyl (DMG).

### Chondroitin Sulfate (CS) Glycosaminoglycans

Provided herein are chondroitin sulfate (CS) glycosaminoglycans including CS disaccharides and polysaccharides. In some embodiment, the chondroitin sulfate is a GlcA-GalN disaccharide. In some embodiments, the GlcA-GalN disaccharide is represented by formula 107:

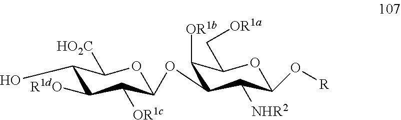

wherein R^{1a}, R^{1b}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine. In some embodiments, R is a fluorous tag. In some embodiments, R^{1a}, R^{1b}, R^{1c}, R^{1d} and/or R² is a protecting group (e.g, hydroxyl or amine protecting group).

In some embodiments, a GlcA-GalN disaccharide is represented by formula 107, wherein R² is SO₃H. In some embodiments, R group of the GlcA-GalN disaccharide of formula 107 is a C1-C12 linear aliphatic primary amine. In some embodiments, R group of the GlcA-GalN disaccharide of formula 107 is a C2-C8 linear aliphatic primary amine. In some embodiments, R group of the GlcA-GalN disaccharide of formula 107 is a C5 linear aliphatic primary amine (—(CH₂)₅NH₂).

In some embodiments, a GlcA-GalN disaccharide is represented by formula 107, wherein R² is an acetyl group (Ac). In some embodiments, R group of the GlcA-GalN disaccharide of formula 107 is a C1-C12 linear aliphatic primary amine. In some embodiments, R group of the GlcA-GalN disaccharide of formula 107 is a C2-C8 linear aliphatic primary amine. In some embodiments, R group of the GlcA-GalN disaccharide of formula 107 is a C5 linear aliphatic primary amine (—(CH₂)₅NH₂).

In some embodiments, in an O-sulfated GlcA-GalN disaccharide is represented by formula 107, wherein R^{1a}, R^{1b}, R^{1c} and R^{1d} are H or SO₃H with the proviso that at least one of R^{1a}, R^{1b}, R^{1c} and R^{1d} is SO₃H. In some embodiments, an O-sulfated GlcA-GalN disaccharide is represented by formula 107, wherein R^{1a}, R^{1b}, R^{1c} and R^{1d} are SO₃H. In some embodiments, R group of the O-sulfated GlcA-GalN disaccharide of formula 107 is a C1-C12 linear aliphatic primary amine. In some embodiments, R group of GlcA-GalN disaccharide of formula 107 is a C2-C8 linear aliphatic primary amine. In some embodiments, R group of the O-sulfated GlcA-GalN disaccharide of formula 107 is a C5 linear aliphatic primary amine (—(CH₂)₅NH₂).

In some embodiments, the N-sulfated, O-sulfated GlcA-GalN disaccharide is represented by formula 107, wherein R² is SO₃H, and wherein R^{1a}, R^{1b}, R^{1c} and R^{1d} are SO₃H.

In some embodiments, R group of GlcA-GalN disaccharide of formula 107 is a C1-C12 linear aliphatic primary amine. In some embodiments, R group of the GlcA-GalN disaccharide of formula 107 is a C2-C8 linear aliphatic primary amine. In some embodiments, R group of GlcA-GalN disaccharide of formula 107 is a C5 linear aliphatic primary amine (—(CH₂)₅NH₂).

In some embodiments, the oligosaccharides of GlcA-GalN disaccharide are represented by formula 108:

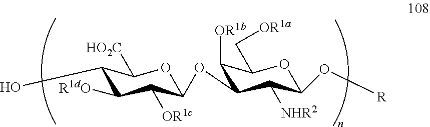

wherein n is any integer ranging from 2 to 200, preferably 20-200; R^{1a}, R^{1b}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine.

In some embodiments, a GlcA-GalN-GlcA-GalN tetrasaccharide is represented by formula 109:

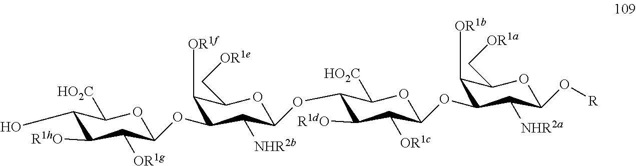

wherein R^{1a}, R^{1b}, R^{1c}, R^{1d}, R^{1e}, R^{1f}, R^{1g}, R^{1h}, R^{2a} and R^{2b} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine. In some embodiments, R is a fluorous tag. In some embodiments, R^{1a}, R^{1b}, R^{1c}, R^{1d}, R^{1e}, R^{1f}, R^{1g}, R^{1h}, R^{2a} and/or R^{2b} is a protecting group (e.g, hydroxyl or amine protecting group).

In some embodiments, a GlcA-GalN-GlcA-GalN tetrasaccharide is represented by formula 109, wherein at least one of R^{2a} and R^{2b} is an acetyl group (Ac). In some embodiments, R^{2a} and R^{2b} are an acetyl group (Ac).

In some embodiments, an N-sulfated GlcA-GalN-GlcA-GalN tetrasaccharide is represented by formula 109, wherein R^{2a} and R^{2b} are H or SO₃H with the proviso that at least one of R^{2a} and R^{2b} is SO₃H. In some embodiments, an N-sulfated GlcA-GalN-GlcA-GalN tetrasaccharide is represented by formula 109, wherein R^{2a} and R^{2b} are SO₃H. In some embodiments, R group of the N-sulfated GlcA-GalN-GlcA-GalN tetrasaccharide of formula 109 is a C1-C12 linear aliphatic primary amine. In some embodiments, R group of the N-sulfated GlcA-GalN-GlcA-GalN tetrasaccharide of formula 109 is a C2-C8 linear aliphatic primary amine. In some embodiments, R group of the N-sulfated GlcA-GalN-GlcA-GalN tetrasaccharide of formula 109 is a C5 linear aliphatic primary amine (—(CH₂)₅NH₂).

In some embodiments, an O-sulfated GlcA-GalN-GlcA-GalN tetrasaccharide is represented by formula 109, wherein R^{1a}, R^{1b}, R^{1c}, R^{1d}, R^{1e}, R^{1f}, R^{1g} and R^{1h}, are H or SO₃H with the proviso that at least one of R^{1a}, R^{1b}, R^{1c}, R^{1d}, R^{1e}, R^{1f}, R^{1g} and R^{1h} is SO₃H. In some embodiments, an O-sulfated GlcA-GalN-GlcA-GalN tetrasaccharide is represented by formula 109, wherein R^{1a}, R^{1b}, R^{1c}, R^{1d}, R^{1e}, R^{1f}, R^{1g} and R^{1h} are SO₃H. In some embodiments, R group of the O-sulfated GlcA-GalN-GlcA-GalN tetrasaccharide of formula 109 is a C1-C12 linear aliphatic primary amine. In some embodiments, R group of the N-sulfated GlcA-GalN-GlcA-GalN tetrasaccharide of formula 109 is a C2-C8 linear aliphatic primary amine. In some embodiments, R group of the N-sulfated GlcA-GalN-GlcA-GalN tetrasaccharide of formula 109 is a C5 linear aliphatic primary amine (—(CH₂)₅NH₂).

In some embodiments, an N-sulfated, O-sulfated GlcA-GalN-GlcA-GalN tetrasaccharide is represented by formula 109, wherein R^{2a} and R^{2b} are H or SO₃H with the proviso that at least one of R^{2a} and R^{2b} is SO₃H, and wherein R^{1a}, R^{1b}, R^{1c}, R^{1d}, R^{1e}, R^{1f}, R^{1g} and R^{1h} are H or SO₃H with the proviso that at least one of R^{1a}, R^{1b}, R^{1c}, R^{1d}, R^{1e}, R^{1f}, R^{1g} and R^{1h} is SO₃H. In some embodiments, the N-sulfated, O-sulfated GlcA-GalN-GlcA-GalN tetrasaccharide is represented by formula 109, wherein R^{2a} and R^{2b} are SO₃H. In some embodiments, the N-sulfated, O-sulfated GlcA-GalN-GlcA-GalN tetrasaccharide is represented by formula 109, wherein R^{1a}, R^{1b}, R^{1c}, R^{1d}, R^{1e}, R^{1f}, R^{1g} and R^{1h} are SO₃H. In some embodiments, R group of the N-sulfated, O-sulfated GlcA-GalN-GlcA-GalN tetrasaccharide of formula 109 is a C1-C12 linear aliphatic primary amine. In some embodiments, R group of the N-sulfated GlcA-GalN-GlcA-GalN tetrasaccharide of formula 109 is a C2-C8 linear aliphatic primary amine. In some embodiments, R group of the N-sulfated GlcA-GalN-GlcA-GalN tetrasaccharide of formula 109 is a C5 linear aliphatic primary amine (—(CH₂)₅NH₂).

In some embodiments, the oligosaccharides of GlcA-GalN-GlcA-GalN tetrasaccharide are represented by formula 110:

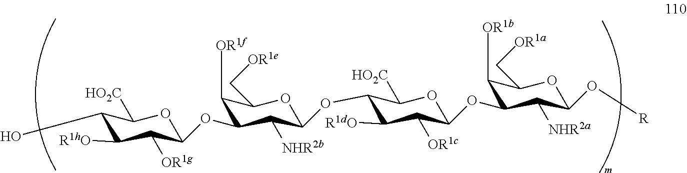

wherein m is any integer ranging from 1 to 100; R^{1a}, R^{1b}, R^{1c}, R^{1d}, R^{1e}, R^{1f}, R^{1g} and R^{1h} are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

In some embodiments, an oligosaccharide of GlcA-GalN-GlcA-GalN tetrasaccharide is represented by formula 110, wherein at least one of R^{2a} and R^{2b} in 1-100 monomeric tetrasaccharide unit of formula 110 is an acetyl group (Ac). In some embodiments, R^{2a} and R^{2b} in 1-100 monomeric tetrasaccharide unit of formula 110 are an acetyl group (Ac).

In some embodiments, an N-sulfated oligosaccharide of GlcA-GalN-GlcA-GalN tetrasaccharide is represented by formula 110, wherein R^{2a} and R^{2b} each monomeric tetrasaccharide unit of formula 110 are each independently H or SO₃H with the proviso that at least one of R^{2a} and R^{2b} is SO₃H.

In some embodiments, an N-sulfated oligosaccharide of GlcA-GalN-GlcA-GalN tetrasaccharide is represented by formula 110, wherein R^{2a} and R^{2b} in 1 to 100 of monomeric units of formula 110 are SO₃H.

In some embodiments, R group of the N-sulfated oligosaccharide of GlcA-GalN-GlcA-GalN tetrasaccharide of formula 110 is a C1-C12 linear aliphatic primary amine. In some embodiments, R group of the oligosaccharide of GlcA-GalN-GlcA-GalN tetrasaccharide of formula 110 is a C2-C8 linear aliphatic primary amine. In some embodiments, R group of the oligosaccharide of GlcA-GalN-GlcA-GalN tetrasaccharide of formula 110 is a C5 linear aliphatic primary amine (—(CH₂)₅NH₂).

In some embodiments, an O-sulfated oligosaccharide of GlcA-GalN-GlcA-GalN tetrasaccharide is represented by formula 110, wherein R^{1a}, R^{1b}, R^{1c}, R^{1d}, R^{1e}, R^{1f}, R^{1g} and R^{1h} in each monomeric tetrasaccharide unit of formula 110 are each independently are H or SO₃H with the proviso that at least one of R^{1a}, R^{1b}, R^{1c}, R^{1d}, R^{1e}, R^{1f}, R^{1g} and R^{1h} is SO₃H. In some embodiments, an O-sulfated oligosaccharide of GlcA-GalN-GlcA-GalN tetrasaccharide is represented by formula 110, wherein R^{1a}, R^{1b}, R^{1c}, R^{1d}, R^{1e}, R^{1f}, R^{1g} and R^{1h} are SO₃H.

In some embodiments, R group of the oligomer of GlcA-GalN-GlcA-GalN tetrasaccharide of formula 110 is a C1-C12 linear aliphatic primary amine. In some embodiments, R group of the oligomer of GlcA-GalN-GlcA-GalN tetrasaccharide of formula 110 is a C2-C8 linear aliphatic primary amine. In some embodiments, R group of the oligomer of GlcA-GalN-GlcA-GalN tetrasaccharide of formula 110 is a C5 linear aliphatic primary amine (—(CH₂)₅NH₂).

In some embodiments, an oligomer of N-sulfated, O-sulfated GlcA-GalN-GlcA-GalN tetrasaccharide is represented by formula 110, wherein R^{2a} and R^{2b} are H or SO₃H with the proviso that at least one of R^{2a} and R^{2b} is SO₃H, and wherein R^{1a}, R^{1b}, R^{1c}, R^{1d}, R^{1e}, R^{1f}, R^{1g} and R^{1h} are H or SO₃H with the proviso that at least one of R^{1a}, R^{1b}, R^{1c}, R^{1d}, R^{1e}, R^{1f}, R^{1g} and R^{1h} is SO₃H. In some embodiments, the N-sulfated, O-sulfated oligomer of GlcA-GalN-GlcA-GalN tetrasaccharide is represented by formula 110, wherein R^{2a} and R^{2b} in 1-100 monomeric tetrasaccharide unit of formula 110 are SO₃H. In some embodiments, the N-sulfated, O-sulfated oligomer of GlcA-GalN-GlcA-GalN tetrasaccharide is represented by formula 110, wherein R^{1a}, R^{1b}, R^{1c}, R^{1d}, R^{1e}, R^{1f}, R^{1g} and R^{1h} in 1-100 monomeric tetrasaccharide unit of formula 110 are SO₃H. In some embodiments, R group of the N-sulfated, O-sulfated oligomer of GlcA-GalN-GlcA-GalN tetrasaccharide of formula 110 is a C1-C12 linear aliphatic primary amine. In some embodiments, R group of the N-sulfated oligomer of GlcA-GalN-GlcA-GalN tetrasaccharide of formula 110 is a C2-C8 linear aliphatic primary amine. In some embodiments, R group of the N-sulfated oligomer of GlcA-GalN-GlcA-GalN tetrasaccharide of formula 110 is a C5 linear aliphatic primary amine (—(CH₂)₅NH₂).

In some embodiments, the CS disaccharides and polysaccharides are 4-O-sulfated. In some embodiments, the 4-O-GlcA-GalN disaccharide is represented by formula 107a:

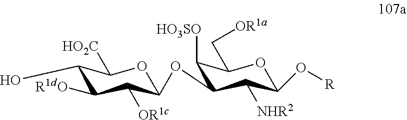

wherein R^{1a}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine.

In some embodiments, the 4-O-GlcA-GalN disaccharide is represented by formula 107b:

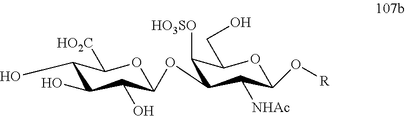

wherein R is H, a C1-C12 linear or branched, substituted or unsubstituted aliphatic primary or secondary amine.

In some embodiments, the 4-O-GlcA-GalN disaccharide is represented by formula 107c:

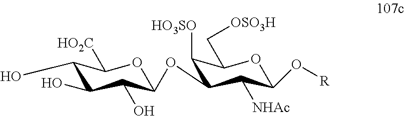

wherein R is H, a C1-C12 linear or branched, substituted or unsubstituted aliphatic primary or secondary amine.

In some embodiments, the 4-O-GlcA-GalN disaccharide is represented by formula 107d:

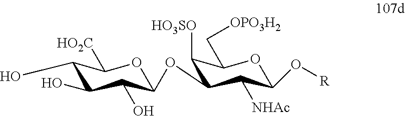

wherein R is H, a C1-C12 linear or branched, substituted or unsubstituted aliphatic primary or secondary amine.

In some embodiments, the oligosaccharides of 4-O-sulfated GlcA-GalN disaccharide are represented by formula 108a:

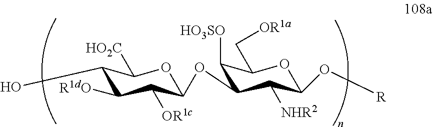

wherein n is any integer ranging from 2 to 200, preferably 20-200; R^{1a}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine.

In some embodiments, the 4-O-GlcA-GalN disaccharide is represented by formula 108b:

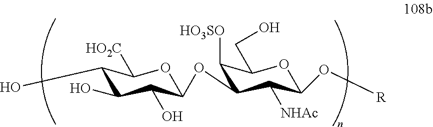

wherein R is H, a C1-C12 linear or branched, substituted or unsubstituted aliphatic primary or secondary amine; n is any integer ranging from 20 to 200.

In some embodiments, the 4-O-GlcA-GalN disaccharide is represented by formula 108c:

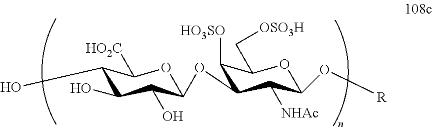

wherein R is H, a C1-C12 linear or branched, substituted or unsubstituted aliphatic primary or secondary amine; n is any integer ranging from 20 to 200.

In some embodiments, the 4-O-GlcA-GalN disaccharide is represented by formula 108d:

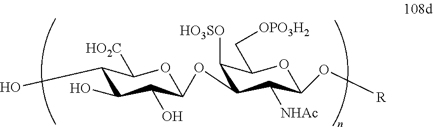

wherein R is H, a C1-C12 linear or branched, substituted or unsubstituted aliphatic primary or secondary amine; n is any integer ranging from 20 to 200.
Treating Neurological Diseases with 4-O-Sulfated Chondroitin Sulfate

Disclosed herein also includes methods and compositions of treating a neuropsychiatric and neurodegenerative disease using the sulfated glycosaminoglycans described herein. In some embodiments, the method comprises administrating to a subject in need a therapeutically effective amount of a composition comprising an effective amount of a 4-O-sulfated chondroitin sulfate polysaccharide, thereby treating the neurological disease or disorder in the subject.

### Overview

Glycans, such as nucleic acids and proteins, are ubiquitous in nature and play crucial roles in biological processes such as development, host-pathogen interactions, and immune regulation. The mammalian central and peripheral nervous systems provide a rich source of diverse glycans. Large-scale analyses have revealed spatial and temporal variations in glycan expression across the brain and identified over 4,000 N-glycosylation sites on more than 1,500 glycoproteins. Despite being the most structurally diverse and rapidly evolving class of macromolecules, glycans remain understudied, and their biological functions in the nervous system are insufficiently understood.

In the brain, a complex meshwork of interwoven glycans and proteins in the extracellular matrix (ECM) provides structural support and mediates important neural functions. Lattice-like ECM structures known as perineuronal nets (PNNs) have been proposed to act as extracellular scaffolds for ligands and to stabilize synapses, thereby modulating brain plasticity and physiological processes. Emerging evidence suggests that PNNs contribute not only to critical period plasticity during development but also to learning and memory processing, psychiatric diseases, drug addiction, and neurodegeneration in adulthood. PNNs predominantly surround parvalbumin-expressing (PV⁺), fast-spiking inhibitory interneurons in cortical areas. They also enwrap excitatory pyramidal neurons in brain regions important for emotional learning and memory, such as the amygdala, entorhinal cortex, and area cornu ammonis 2 (CA2).

The CA2 subregion of the hippocampus has unique molecular, synaptic, and morphological characteristics and is essential for social recognition memory. Consistent with this function, alterations in the cellular structure and circuitry in the area CA2 have been observed in neuropsychiatric disorders associated with cognitive and social dysfunction. Although recent studies suggest that PNNs in the CA2 can contribute to both PV⁺ interneuron and excitatory pyramidal neuron plasticity, the molecules and mechanisms that regulate PNNs and CA2-dependent functions such as social memory remain unclear.

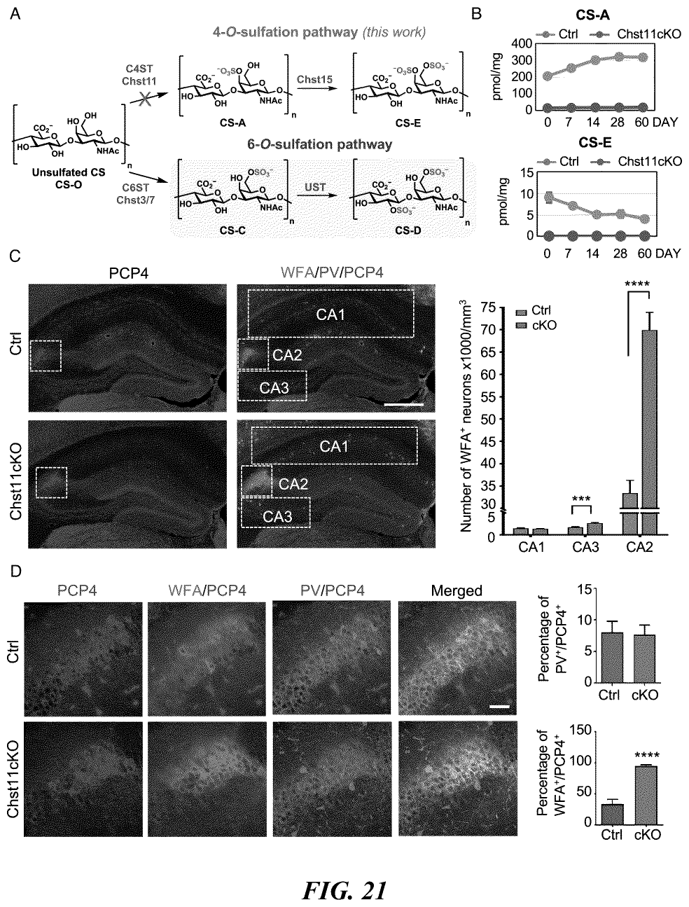

PNNs are highly enriched in chondroitin sulfate proteoglycans (CSPGs), a series of core proteins decorated with chondroitin sulfate (CS) polysaccharides. The repeating D-glucuronic acid (GlcA) and N-acetyl-D-galactosamine (GalNAc) disaccharide units of CS polysaccharide chains display a variety of sulfation motifs (see, for example, FIG. 21, panel A) that dynamically change throughout development into adulthood. Notably, increased expression of the 4-O-sulfated motif CS-A coincides with PNN maturation and the end of critical period plasticity. By adulthood, the CS-A motif represents nearly 90% of the total CS in the brain. The 4-O-sulfated CS-A and CS-E motifs have also been reported to play important roles in axon regeneration after central nervous system (CNS) injury. These motifs are up-regulated in the glial scar upon injury and limit axon regeneration by interacting with protein receptors such as the tyrosine phosphatase receptor a (PTPa) and Nogo receptor. Loss of 4-O-sulfated CS motifs via knockdown of the chondroitin-4-O-sulfotransferase Chst11 in zebrafish enhanced regeneration after spinal cord injury. Based on these and other observations, chondroitin 4-O-sulfation has traditionally been viewed as a “molecular brake” that inhibits neuroplasticity. However, its functions in PNNs and in the uninjured adult brain have not been directly investigated.

Studies aimed at understanding PNNs have employed either chondroitinase ABC (ChABC) to enzymatically digest CS polysaccharides or transgenic mice lacking PNN proteins such as tenascin-R and link protein (HAPLN1). While these approaches have revealed important insights, they also drastically disrupt PNNs and render them indistinguishable from the diffuse ECM. Modulation of the sulfation patterns on CSPGs provides a less perturbative, complementary approach to manipulate and study PNNs. For example, overexpression of chondroitin-6-O-sulfotransferase-1 (C6ST-1) in transgenic mice modulated 6-O-sulfation and PNN formation in the developing visual cortex (VC), leading to persistent ocular dominance plasticity. However, the role of CS sulfation in PNNs has been examined primarily in this context of 6-O-sulfotransferase overexpression.

Presented herein includes the disclosure related to the impact of CS 4-O-sulfation, the dominant form of sulfation and the most abundant glycosaminoglycan structure in the adult mammalian brain, on PNNs, plasticity, and higher-order brain functions such as mood and cognition. The data presented herein demonstrate that brain-specific deletion of the chondroitin 4-O-sulfotransferase gene Chst11 in mice significantly perturbed PNN levels surrounding excitatory CA2 pyramidal neurons. The resulting increase in PNNs led to reduced CREB activation, an imbalance of excitatory and inhibitory synapses, as well as anxiety and social memory dysfunction—phenotypes that were rescued by treatment with ChABC or 4-O-sulfated CS polysaccharides. In agreement with these findings, a chemical inhibitor used to reduce CS 4-O-sulfation levels recapitulated the malformation of PNNs and synaptic defects in hippocampal neurons. Moreover, viral-mediated CA2 region-specific deletion of Chst11 in adult mice also increased PNN densities, inhibited CREB activity, and impaired social memory. Together, these data reveal important roles for CS 4-O-sulfation in adult brain plasticity, social memory, and anxiety regulation, and they suggest CS polysaccharides as targets for the study and potential treatment of neurological diseases characterized by mood disorders and social dysfunction.

4-O-sulfated chondroitin sulfate (CS)

Disclosed herein includes methods and compositions using 4-O-sulfated CS for the treatment of a neurological disease or disorder. In some embodiments, the 4-O-sulfated CS has a general formula represented by formula 108a:

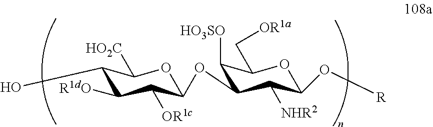

wherein n is any integer ranging from 2 to 200, preferably 20-200; R^{1a}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine.

In some embodiments, the 4-O-GlcA-GalN disaccharide is represented by formula 108b:

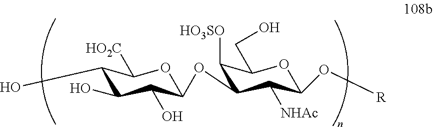

wherein R is H, a C1-C12 linear or branched, substituted or unsubstituted aliphatic primary or secondary amine; n is any integer ranging from 20 to 200.

In some embodiments, the 4-O-GlcA-GalN disaccharide is represented by formula 108c:

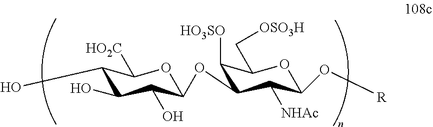

wherein R is H, a C1-C12 linear or branched, substituted or unsubstituted aliphatic primary or secondary amine; n is any integer ranging from 20 to 200.

### Compositions and Administration

Some embodiments provided herein are directed to an effective amount of a pharmaceutical composition comprising the sulfated polysaccharides (e.g., 4-O-sulfated CS polysaccharides) and at least one pharmaceutically acceptable carrier or excipient. In some embodiments, the pharmaceutical composition comprises a 4-O-sulfated CS polysaccharide having a formula of 108a, 108b, or 108c and at least on pharmaceutically acceptable carrier or excipient.

Carriers or excipients can be used to produce compositions. The carriers or excipients can be chosen to facilitate administration of the compound or composition. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

Suitable dosage forms, in part, depend upon the use or the route of administration, for example, oral, transdermal, transmucosal, inhalant, orby injection (parenteral). Such dosage forms should allow the compound to reach target cells. Other factors include considerations such as toxicity and dosage forms that retard the compound or composition from exerting its effects.

The compound or composition can be administered by different routes including intravenous, intraperitoneal, subcutaneous, intramuscular, oral, transmucosal, rectal, transdermal, or inhalant. In some embodiments, the composition can be administered by oral administration. For oral administration, for example, the compound or composition can be formulated into conventional oral dosage forms such as capsules, tablets, and liquid preparations such as syrups, elixirs, and concentrated drops.

In some embodiments, the compositions or compounds described herein can be administrated to the central nervous system of a subject via, for example, intrathecal injection, intracerebroventricular injection, intraparenchymal injection, perispinal injection, intravenous injection, intraocular injection.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C12). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

Injection (parenteral administration) may be used, e.g., intramuscular, intravenous, intraperitoneal, and/or subcutaneous. For injection, the compound or composition can be formulated in sterile liquid solutions, such as in physiologically compatible buffers or solutions, such as saline solution, Hank's solution, or Ringer's solution. In addition, the compound or composition may be formulated in solid form and redissolved or suspended immediately prior to use. Lyophilized forms can also be produced. In some embodiments, the composition is administered to the subject by intravenous administration. In some embodiments, the composition is formulated for intravenous administration. In some embodiments, the composition is administered intravenously as a bolus, injection, infusion, or prolonged infusion.

### Neurological Disorders

Also provided herein is a method of treating a neurological disease or disorder using the sulfated oligosaccharides such as 4-O-sulfated chondroitin sulfate (CS) polysaccharides described herein. The method can comprise administering the 4-O-sulfated CS polysaccharides described herein to a subject in need thereof. In some embodiments, the disease or disorder is a neurological disease or disorder, a neurodegenerative disease or disorder, and/or a disease or disorder of the central nervous system (CNS).

Various diseases and disorders can be treated with the sulfated polysaccharides provided herein. In some embodiments, the disease or disorder can be a neurological disease or disorder. Neurological diseases or disorders are diseases or disorders of the central and peripheral nervous system including the brain, spinal cord, cranial nerves, peripheral nerves, nerve roots, autonomic nervous system, neuromuscular junction, and muscles. Neurological disorders can include epilepsy, Alzheimer's disease and other dementias, cerebrovascular diseases including stroke, migraine and other headache disorders, multiple sclerosis, Parkinson's disease, neuroinfections, brain tumors, and traumatic disorders of the nervous system due to head trauma.

Exemplary neurological diseases or disorders include, but are not limited to, Absence of the Septum Pellucidum, Acid Lipase Disease, Acute Disseminated Encephalomyelitis, Adrenoleukodystrophy, Agenesis of the Corpus Callosum, Agnosia, Aicardi Goutieres Syndrome Disorder, Aicardi Syndrome, Alexander Disease, Alpers Disease, ALS Amyotrophic Lateral Sclerosis, Alternating Hemiplegia, Alzheimer's Disease, Amyotrophic Lateral Sclerosis ALS, Anencephaly, Angelman Syndrome, Antiphospholipid Syndrome, Aphasia, Apraxia, Arachnoid Cysts, Arachnoiditis, Arteriovenous Malformation, Asperger Syndrome, Ataxia Telangiectasia, Ataxias and Cerebellar or Spinocerebellar Degeneration, Atrial Fibrillation and Stroke, Attention Deficit Hyperactivity Disorder, Autism, Autism Spectrum Disorder, Back Pain, Barth Syndrome, Batten Disease, Behcet's Disease, Bell's Palsy, Benign Essential Blepharospasm, Binswanger's Disease, Brachial Plexus Injuries, Brain and Spinal Tumors, Brown Sequard Syndrome, CADASIL, Canavan Disease, Carpal Tunnel Syndrome, Central Cord Syndrome, Central Pain Syndrome, Central Pontine Myelinolysis, Cephalic Disorders, Cerebellar Degeneration, Cerebellar Hypoplasia, Cerebral Aneurysms, Cerebral Arteriosclerosis, Cerebral Atrophy, Cerebral Cavernous Malformation, Cerebral Hypoxia, Cerebral Palsy, Cerebro Oculo Facio Skeletal Syndrome COFS, Charcot Marie Tooth Disease, Chiari Malformation, Chorea, Chronic Inflammatory Demyelinating Polyneuropathy CIDP, Chronic Pain, Coffin Lowry Syndrome, Colpocephaly, Coma, Complex Regional Pain Syndrome, Congenital Myasthenia, Congenital Myopathy, Corticobasal Degeneration, Craniosynostosis, Creutzfeldt Jakob Disease, Cushing's Syndrome, Dandy Walker Syndrome, Deep Brain Stimulation for Parkinson s Disease, Dementia, Dementia With Lewy Bodies, Dermatomyositis, Developmental Dyspraxia, Diabetic Neuropathy, Dravet Syndrome, Dysautonomia, Dysgraphia, Dyslexia, Dyssynergia Cerebellaris Myoclonica, Dystonias, Empty Sella Syndrome, Encephalitis Lethargica, Encephaloceles, Encephalopathy, Epilepsy, Erb Duchenne and Dejerine Klumpke Palsies, Essential Tremor, Fabry Disease, Fahr s Syndrome, Familial Periodic Paralyses, Farber's Disease, Febrile Seizures, Fibromuscular Dysplasia, Foot Drop, Friedreich's Ataxia, Frontotemporal Dementia, Gaucher Disease, Generalized Gangliosidoses, Gerstmann's Syndrome, Gerstmann Straussler Scheinker Disease, Giant Axonal Neuropathy, Glossopharyngeal Neuralgia, Guillain Barre Syndrome, Headache, Hemicrania Continua, Hemifacial Spasm, Hereditary Neuropathies, Hereditary Spastic Paraplegia, Herpes Zoster Oticus, Holmes Adie syndrome, Holoprosencephaly, Huntington's Disease, Hydranencephaly, Hydrocephalus, Hydromyelia, Hypersomnia, Hypertonia, Hypotonia, Inclusion Body Myositis, Incontinentia Pigmenti, Infantile Neuroaxonal Dystrophy, Infantile Refsum Disease, Infantile Spasms, Inflammatory Myopathies, Iniencephaly, Isaac's Syndrome, Kearns Sayre Syndrome, Kennedy's Disease, Kleine Levin Syndrome, Klippel Feil Syndrome, Klippel Trenaunay Syndrome KTS, Krabbe Disease, Lambert Eaton Myasthenic Syndrome, Landau Kleffner Syndrome, Learning Disabilities, Leigh's Disease, Lennox Gastaut Syndrome, Lesch Nyhan Syndrome, Leukodystrophy, Lipid Storage Diseases, Lipoid Proteinosis, Lissencephaly, Locked In Syndrome, Machado Joseph Disease, Megalencephaly, Melkersson Rosenthal Syndrome, Meningitis and Encephalitis, Menkes Disease, Meralgia Paresthetica, Metachromatic Leukodystrophy, Microcephaly, Migraine, Miller Fisher Syndrome, Mitochondrial Myopathies, Mitochondrial Myopathy, Moebius Syndrome, Monomelic Amyotrophy, Motor Neuron Diseases, Moyamoya Disease, Mucolipidoses, Mucopolysaccharidoses, Multi Infarct Dementia, Multifocal Motor Neuropathy, Multiple Sclerosis, Multiple System Atrophy, Multiple System Atrophy with Orthostatic Hypotension, Muscular Dystrophy, Myasthenia Gravis, Myoclonus, Myopathy, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Sequelae Of Lupus, Neuromyelitis Optica, Neuronal Migration Disorders, Neurosarcoidosis, Neurosyphilis, Neurotoxicity, Niemann Pick Disease, Normal Pressure Hydrocephalus, Occipital Neuralgia, Ohtahara Syndrome, Olivopontocerebellar Atrophy, Opsoclonus Myoclonus, Orthostatic Hypotension, Paraneoplastic Syndromes, Paresthesia, Parkinson's Disease, Paroxysmal Choreoathetosis, Paroxysmal Hemicrania, Parry Romberg, Pelizaeus Merzbacher Disease, Peripheral Neuropathy, Periventricular Leukomalacia, Pervasive Developmental Disorders, Pinched Nerve, Piriformis Syndrome, Pituitary Tumors, Polymyositis, Pompe Disease, Porencephaly, Post Polio Syndrome, Postural Tachycardia Syndrome, Primary Lateral Sclerosis, Progressive Multifocal Leukoencephalopathy, Progressive Supranuclear Palsy, Prosopagnosia, Pseudotumor Cerebri, Psychogenic Movement, Rasmussen's Encephalitis, Refsum Disease, Repetitive Motion Disorders, Restless Legs Syndrome, Rett Syndrome, Reye's Syndrome, Sandhoff Disease, Schilder's Disease, Schizencephaly, Septo Optic Dysplasia, Shaken Baby Syndrome, Shingles, Sjögren's Syndrome, Sleep Apnea, Sotos Syndrome, Spasticity, Spina Bifida, Spinal Cord Infarction, Spinal Cord Injury, Spinal Muscular Atrophy, Stiff Person Syndrome, Striatonigral Degeneration, Stroke, Sturge Weber Syndrome, Subacute Sclerosing Panencephalitis, SUNCT Headache, Swallowing Disorders, Sydenham Chorea, Syncope, Syringomyelia, Tabes Dorsalis, Tardive Dyskinesia, Tarlov Cysts, Tay Sachs Disease, Tethered Spinal Cord Syndrome, Thoracic Outlet Syndrome, Thyrotoxic Myopathy, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis Syndromes of the Central and Peripheral Nervous Systems, Von Hippel Lindau Disease VHL, Wallenberg s Syndrome, Wernicke Korsakoff Syndrome, Whiplash, Whipple's Disease, Williams Syndrome, Wilson Disease, and Zellweger Syndrome.

In some embodiments, a disease or a disorder is a neurodegenerative disease or disorder. Neurodegenerative diseases or disorders are a heterogeneous group of disorders that are characterized by the progressive degeneration of the structure and function of the central nervous system or peripheral nervous system. In some embodiments, neurodegenerative diseases are diseases marked by continuous and progressive deterioration of the function of neural cells which are not caused by any underlying trauma or infection. Exemplary neurodegenerative diseases or disorders include, but are not limited to, Alzheimer's disease (AD), Huntington's disease (HD), Parkinson's disease (PD), amyotrophic lateral sclerosis (ALS), and prion diseases.

In some embodiments, a disease or a disorder is a disease or condition of the central nervous system (CNS). Exemplary disease or a condition of the CNS include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome.

In some embodiments, the CNS disease is a movement disorder, a memory disorder, addiction, attention deficit/hyperactivity disorder (ADHD), autism, bipolar disorder, depression, encephalitis, epilepsy/seizure, migraine, multiple sclerosis, a neurodegenerative disorder, a psychiatric disease, a neuroinflammatory disease, Alzheimer's disease, Huntington's disease, Parkinson's disease, Tourette syndrome, dystonia, or a combination thereof. In some embodiments, the disease is a neuroinflammatory disease. For example, the neuroinflammatory disease is Parkinson's disease, Alzheimer's disease, multiple sclerosis, or a combination thereof. In some embodiments, the CNS disease is Huntington's disease.

In some embodiments, the disease or disorder is Alzheimer's disease, bipolar disorder, schizophrenia, autism, fragile X syndrome, Rett syndrome, anxiety or anxiety-related disorders, and social memory dysfunction. In some embodiments, the subject is diagnosed with mild cognitive impairment or dementia associated with Alzheimer's disease.

### EXAMPLES

Some aspects of the embodiments discussed above are disclosed in further detail in the following examples, which are not in any way intended to limit the scope of the present disclosure.

### Example 1

### Materials and Methods

This Example describes general materials, methods, and synthetic procedures used in Examples 2-8 below.

Unless stated otherwise, reactions were performed in oven-dried glassware under an argon atmosphere using freshly dried solvents. Solvents were dried via passage through an activated alumina column under argon. All other commercially obtained reagents were used as received unless otherwise noted. Thin-layer chromatography (TLC) was performed using E. Merck silica gel 60 F254 pre-coated plates (0.25 mm). Visualization of the chromatogram was accomplished by UV, cerium ammonium molybdate, or ninhydrin staining as necessary. ICN silica gel (particle size 0.032-0.063 mm) was used for column chromatography. ¹H NMR (Varian Inova-500 MHz and Bruker-400 MHz), and ¹³C NMR (Varian Inova-125 MHz and Bruker-100 MHz) spectra were recorded using CDC₃, CD3COCD3, CD30D, or D2O as solvent(s). Data for ¹H are reported as follows: chemical shift (d ppm), multiplicity (s=singlet, bs=broad singlet, d=doublet, t=triplet, q=quartet, m=multiplet), coupling constant in Hz, and integration. When necessary, proton and carbon assignments were made by means of ¹H-¹H gCOSY, ¹H-¹³C gHSQCAD and ¹H-¹³C gHMBCAD. Mass spectra were obtained using a Waters LCT Premier XE high-resolution mass spectrometer at the California Institute of Technology. Sodium heparinate was obtained from Smithfield Bioscience. Abbreviations: fluorenylmethyloxycarbonyl (Fmoc); benzenethiol (SPh); levulinoyl (Lev); 2-naphthyl (Naph); (2-naphthyl)methyl (Nap); trifluoroacetic anhydride (TFAA); trifluoroacetate (TFA); uronic acid (UA).

### General Synthetic Procedures

General procedure of fluorous solid phase extraction (FSPE) for non-sulfated compounds. The crude residue (less than 0.20 g) was dissolved in 0.80 mL of DMF and loaded onto the cartridge filled with fluorous silica gel (10 g). Non-fluorous compounds were eluted with 20 mL of McOH/H₂O (3:2, v/v) and 10 mL of McOH/H₂O (7:3, v/v). The fluorous compounds were eluted with 20 mL of acetone, and the fluorous fraction was concentrated to obtain the desired product.

General procedure of FSPE for O-sulfation step. The crude reaction mixture (less than 0.20 g) in DMF (0.50 mL) was loaded onto the cartridge filled with fluorous silica gel (10 g). Non-fluorous compounds were eluted with 20 mL of MeOH/H2O (1:1, v/v), and fluorous compounds were eluted with 20 mL of acetone. The fluorous fraction was collected and passed through a column of Dowex® 50W X8 Na⁺ resin. The resulting solution was concentrated to obtain the desired compound as the sodium salt.

General procedure of FSPE for hydrolysis, N3 to NH2, N-acetylation, and N-sulfation steps. The crude reaction mixture (less than 0.20 g) was dissolved in a mixture of MeOH and water (1:1, v/v, 1.0 mL) and loaded onto the cartridge filled with fluorous silica gel (10 g). Non-fluorous compounds were eluted with 20 mL of MeOH/H₂O (3:7, v/v), and fluorous compounds were eluted with 20 mL of MeOH. The fluorous fraction was collected and passed through a column of Dowex® 50W X8 Na⁺ resin. The resulting solution was concentrated to obtain the desired compound as the sodium salt.

N3 to NHAc. To a solution of starting material (0.040 M) in pyridine thioacetic acid (130 eq.) was added at RT. After stirring for 18 h, the mixture was diluted with DCM and washed with saturated aq. NaHCO₃ and then brine. The organic layer was dried over anhydrous Na₂SO₄, filtered, and concentrated. The resulting residue was purified by silica gel column chromatography (elution with 3:2® 1:2 hexane/EtOAc) to obtain the desired compound.

Cleavage of Fmoc group. Piperidine (10 equiv per Fmoc group) was added to a solution of starting material (0.050 M) in DCM at RT. Stirring was continued until TLC (hexane/EtOAc, 2:1, v/v) indicated disappearance of starting material (˜1 h). The solvent was then removed under reduced pressure. The resulting crude material was purified by FSPE for non-sulfated compounds. The fluorous fraction was concentrated to obtain the desired compound.

Cleavage of Lev group. A solution of starting material (0.030 M) and hydrazine acetate (10 equiv per Lev group) in CH2Cl2/CH3OH (9:1, v/v) was stirred at RT. Stirring was continued until TLC (hexane/EtOAc, 2:1, v/v) indicated disappearance of starting material (˜1 h). The solvent was then removed under reduced pressure. The resulting crude material was purified by FSPE for non-sulfated compounds. The fluorous fraction was concentrated to obtain the desired compound.

Cleavage of TBDPS group. To a solution of starting material (0.020 M) in pyridine (1.6 mL) in a plastic conical tube was added hydrogen fluoride pyridine (0.60 mL) at 0° C. After stirring at RT for 2 h, the reaction was quenched with saturated aq. NaHCO₃. The mixture was then extracted with EtOAc, and the organic layer was dried over anhydrous Na₂SO₄, filtered, and concentrated. The resulting crude material was purified by FSPE for nonsulfated compounds. The fluorous fraction was concentrated to obtain the desired compound.

Cleavage of Nap group. DDQ (10 equiv per Nap group) was added to a solution of starting material (8.0 mM) in DCE/MeOH/PBS (16:4:0.25, v/v/v). Stirring was continued until TLC (hexane/EtOAc, 1:1, v/v) indicated disappearance of starting material (˜8 h). The reaction mixture was diluted with DCM, and the organic layer was washed with saturated aq. NaHCO₃, dried over anhydrous Na₂SO₄, filtered, and concentrated. The resulting crude material was purified by FSPE for non-sulfated compounds. The fluorous fraction was concentrated to obtain the desired compound.

O-Sulfation. A solution of the starting material (0.050 M) and SO₃·Et₃N (10 equiv per OH group) in DMF was stirred at 50° C. under argon atmosphere. Stirring was continued until TLC (DCM/MeOH, 7:1, v/v) indicated completion of the reaction (˜8-10 h). A mixture of triethylamine and MeOH (1:1, v/v, 0.20 mL) was added, and stirring was continued for 30 min. The mixture was directly transferred to a fluorous silica column and purified by FSPE.

Hydrolysis. The starting material (0.010 M) was dissolved in a mixture of THF and MeOH (1:1, v/v, 2 mL), and 1.0 M aq. LiOH (1.0 mL) was added at RT. The reaction was stirred at RT for 1 h and then at 40° C. until TLC (EtOAc:pyridine:H₂O:AcOH, 12:5:3:1, v/v) indicated completion of the reaction (˜24-36 h). After completion, 1.0 M aq. NH4Cl (1.0 mL) was added to quench the reaction, and the reaction mixture was concentrated under reduced pressure to remove THF. The resulting solution was transferred to a fluorous silica column and purified by FSPE. The fluorous fraction was collected and passed through a column of Dowex® 50W X8 Na⁺ resin. The resulting solution was concentrated to obtain the desired compound as sodium salt.

N3 to NH2. To a solution of the starting material (0.020 M) in THF (1.0 mL) was added aqueous NaOH (0.10 M, 0.45 mL) and PMe3 in THF (1.0 M solution, 10 equiv per N3 group). The reaction mixture was stirred at RT until completion of the reaction (˜1 h), as determined by MALDI-TOF. The reaction mixture was concentrated to remove THF, and the residue was purified by FSPE to obtain the desired compound.

N-Acetylation. The starting material (0.020 M) was dissolved in a mixture of anhydrous MeOH (1.0 mL) and Et₃N (30 equiv per NH2 group), to which was added acetic anhydride (20 equiv per NH2 group). The reaction mixture was stirred for 3 h at RT and concentrated under reduced pressure. The residue was purified by FSPE to obtain the desired compound.

N-Sulfation. To a solution of the starting material (0.020 M) in MeOH (1.0 mL) and trifluoroethane (0.15 mL) were added trimethylamine (0.15 mL), aqueous NaOH (0.10 M, 0.40 mL) and SO₃·Py (15 equiv per NH2 group). After 1 hour, another batch of S03 Py (15 equiv per NH2 group) was added, and stirring was continued until TLC (EtOAc:pyridine:H₂O:AcOH, 8:5:3:1, v/v) indicated completion of the reaction (˜12 h). The reaction mixture was then concentrated under reduced pressure, and the residue was purified by FSPE to obtain the desired compound.

Hydrogenation. The starting material was dissolved in a mixture of tert-butanol and H₂O (1:1, v/v, 8.0 mL) to which Pd(OH)₂ on carbon (20%, 4 times the weight of starting material) was added. The resulting mixture was placed under an atmosphere of hydrogen and the reaction progress was monitored by Maldi-TOF. After 16 hours, Pd(OH)₂ on carbon (20%, 4 times the weight of starting material) was added. After completion of the reaction, the mixture was filtered, and the residue was washed with a mixture of 1:1 tert-butanol/H₂O (8.0 mL). The combined solvents were concentrated under reduced pressure, concentrated, and the resulting residue was purified by Sephadex G15 (elution with H₂O) to give target HS tetrasaccharide.

### Glycan Microarray

Materials. Dulbecco's phosphate buffered saline (DPBS), 1× was purchased from Corning, bovine serum albumin (BSA) from Fisher BioReagents. Tris-buffered saline with 0.1% Tween-20 (TBST) was prepared by mixing 10×TBS (0.10 L, Fisher BioReagents), Tween-20 (1.0 g, Millipore Sigma) and ddH₂O (0.90 L). Alexa Fluor 647-conjugated streptavidin was purchased from Invitrogen. Recombinant human his-tagged FGF2 protein (>95% purity) was purchased from Sino Biological. 6×-His Tag monoclonal antibody AF647 was purchased from ThermoFisher. Recombinant human FGF4 (>95% purity), biotinylated anti-human FGF4 antibody (>95% purity), recombinant human IL-8 (CXCL8) (77 a.a.) (>95% purity), and biotinylated anti-human IL-8 antibody (CXCL8) (>95% purity) were purchased from Peprotech. Human FGF2, FGF4, and CXCL8 was reconstituted in 0.1% BSA in PBS before use or as recommended by supplier. Schott NEXTERION® 3-D Hydrogel coated microarray slides were purchased from Applied Microarrays Inc.

Heparan sulfate tetrasaccharide microarray fabrication. HS oligosaccharides were dissolved in sodium phosphate buffer (pH 8.5, 50 mM) in 9 concentrations: 200 μM, 100 μM, 50 μM, 25 μM, 12.5 μM, 6.25 μM, 3.13 μM, 1.57 μM, 0.79 μM. The robotic arrayer SX (from Scienion, Berlin, Germany) delivered 414 pL of the solution containing oligosaccharides (9 spots per concentration) to the array slides using dispensing nozzle PDC80, P-2040, under ˜50% relative humidity at 20° C. The array spots had an average diameter of about 70 μm with a distance of 260 μm between the centers of adjacent spots. The slides were incubated in a chamber with saturated (NH₄)₂SO₄ solution (81% relative humidity) for 24 h at 20° C., and unreacted HS oligosaccharides were removed from the slides by rinsing with deionized water. The remaining N-hydroxysuccinimide (NHS) ester groups were blocked by placing slides in a solution containing 50 mM ethanolamine in PBST (137 mM NaCl, 13 mM Na₂HPO₄, 1.6 mM NaH₂PO₄, 2.7 mM KCl, 0.010% Tween-20) at 50° C. for 1.5 h. Slides were rinsed several times with deionized water, dried by centrifugation, and stored before use.

HS tetrasaccharide microarray binding assay and array slide processing. The slide was blocked with 10% BSA in TBST (4.0 mL) with gentle rocking at RT for 1 h, followed by submerging in TBST. This condition was empirically determined to reduce nonspecific binding of proteins to the microarray and to provide optimal signal-to-noise ratios. Next, 5.0 μL of the primary detection protein (2.5 μg, 0.50 μg/mL) solution was transferred to 2.5 mL of TBST w/1% BSA in a chamber and the slide was incubated with gentle shaking for 1 h at RT. The slide was then washed two times for 5 min each in TBST (4.0 mL) while gently rocking. After the washes, the slide was incubated with the appropriate biotinylated antibody (3.0 μg, 0.50 μg/mL) in 1% BSA in TBST solution (3.0 mL) with gentle rocking for 1 h. The slide was then washed two times for 5 min in TBST (5.0 mL) while gently rocking. Next, the slide was incubated with 4.0 mL of Alexa Fluor 647-conjugated streptavidin detection solution (1.0 μg/mL in TBST w/1% BSA) in the dark with gentle rocking for 1 h, washed with TBST (5.0 mL, two times) followed by PBS (45 mL, two times) and ddH₂O (45 mL, two times), and then dried under a gentle stream of 0.2 micron-filtered air. For his-tagged FGF2, detection was accomplished with an AF647-labeled anti-his antibody. All incubations and washes were carried out at RT unless otherwise noted. The microarray was scanned and analyzed at 647 nm using an Agilent G2565CA Microarray Scanner, and fluorescence quantification was performed using ImageJ software with correction for local background. To ensure accurate signal quantification, the average signal intensity was calculated over a fixed area for all spots. Results were plotted with Prism 9 software at a single concentration as background corrected fluorescence values normalized to the compound with the highest signal. Bar graphs represent the mean±SD for each compound (50 μM) in nonuplicate. Each experiment was performed 2-3 times. Heat maps were generated in Excel with columns sorted by sulfation status of the amino positions and rows sorted by sulfation status of the 2-O- and 6-O-hydroxyl groups.

Sulfation Logo Calculations. The sulfation logos were calculated based on methods used to determine DNA consensus sequences for transcription factors that capture enrichment and depletion at each position. Each tetrasaccharide was assigned a 6-letter code representing sulfation (S), acetylation (A) or no modification (H) at each position. To generate the logos, the “relative number of binding events” for each compound was calculated by rounding the normalized microarray binding data from the heatmaps down to the nearest integer. This foreground data set was considered to be the number of times a tetrasaccharide sequence appeared in the list of bound sequences and was used to generate a position frequency matrix, where all bound sequences were coded (as described above), and a sum representing the observed frequency of each modification at each position was obtained. From this, a position weight matrix (PWM) was generated by taking the log of the ratio of the observed frequency to the expected frequency. Because our 64-compound library is comprehensive, the modifications at each position are represented with equal frequency, and the expected frequency for all modifications is 0.5. From the PWMs, sequence logos were visualized as the log likelihood for each modification at each position. The code for generating these logos is publicly available (DOI:10.5281/zenodo.7787616).

### Example 2

### General Synthetic Strategy of the Library

Provided in this Example is a general strategy of synthesizing the HS tetrasaccharide library.

In contrast to peptides and nucleic acids, universal building blocks for the synthesis of GAGs have not been developed. Moreover, the majority of the synthesis is dedicated to preparing suitably protected mono- and disaccharide precursors. For example, starting from commercially available monosaccharides, 18-30 chemical steps are typically required to produce each HS disaccharide used for glycosylation. Ideally, a small set of building blocks would be employed to generate a large number of different sulfation motifs. Towards this end, tetrasaccharide 65 was designed as a versatile building block (FIG. 1B). The IdoA-containing backbone glucosamine (GlcN)-IdoA-GlcN-IdoA was chosen because the conformational flexibility of IdoA is crucial for many HS-protein interactions and often induces a kink in the HS structure that is important for protein binding. A tetrasaccharide was selected because four monosaccharide residues are typically the minimum length required to engage high-affinity binding sites in many proteins, including fibroblast growth factors (FGFs) and chemokines such as CCL5. Furthermore, a comprehensive library of sulfation sequences at the major positions (N-, 2-O- and 6-O-) would consist of 64 tetrasaccharides, an ambitious yet achievable goal for chemical synthesis and biological investigations.

To facilitate library synthesis, the 2-0, 6-0 and N-positions in tetrasaccharide 65 were differentially protected with six orthogonal functionalities (FIG. 1B). The orthogonality of these functional groups allows for their selective removal to unmask each position for sulfation. Site-specific O-deprotection steps, followed by O-sulfation, would install sulfate groups at the desired 2-O- and 6-O-positions (FIG. 1C). Hydrolysis to saponify the methyl esters and selectively remove the N-trifluoroacetyl (N-TFA) groups, followed by N-sulfation or N-acetylation, would provide different amino group modifications in the tetrasaccharide. Thus, a single universal building block would enable access to all 64 sequences (1-64), the largest number of sulfation motifs generated from a single HS precursor so far.

A major challenge to HS library synthesis is the substantial number of late-stage modification steps (274 steps for 64 tetrasaccharides). Each step also requires the labour-intensive, multi-step purification of highly polar, charged intermediates using a combination of silica gel, size-exclusion and/or strong anion-exchange chromatography. Fluorous chemistry was used to overcome these challenges. Although fluorous-assisted synthesis has been elegantly applied to many organic compounds including a limited number of glycans, it has not been extensively exploited for HS synthesis. Fluorous chemistry would greatly accelerate the synthesis of large GAG libraries by reducing the purification of highly charged intermediates to a single FSPE step. Moreover, fluorous-assisted synthesis would afford the efficiency, convenience and flexibility of solution-phase synthesis by permitting easy reaction monitoring using standard spectroscopic methods (for example, thin-layer chromatography (TLC), MS and NMR) and by circumventing challenges associated with solid-phase synthesis (for example, solvent constraints for resin swelling and difficult reaction monitoring). Finally, fluorous-assisted synthesis is amenable to automation, which could facilitate the first automated synthesis of HS oligosaccharides larger than disaccharides. a C₆F₁₃ fluorous tag was thus appended to the reducing end of tetrasaccharide 65 (FIG. 1B) via a benzyloxycarbonyl (Cbz)-like aminopentyl linker, which, upon cleavage, would expose an amine handle for versatile conjugation of the tetrasaccharides to small molecules, polymers, proteins and microarray surfaces.

### Example 3

### Synthesis of Tetrasaccharide 65

Provided in this Example is a general strategy of synthesizing tetrasaccharide 65.

Synthesis of 65 began with the controlled hydrolysis of heparin using aqueous triflic acid, followed by esterification, NH₂-to-N₃ conversion, and acetylation to give the peracetylated disaccharide 66 in 20% overall yield (FIG. 2). The use of disaccharides derived from natural heparin greatly simplified the synthesis by eliminating the need to synthesize IdoA monosaccharides and to perform the challenging 1,2-cis glycosylation reaction. Disaccharide 66 was elaborated to form 68 in a total of only nine steps and five purifications from heparin, about half the number of steps previously required. Compound 68 is a versatile intermediate that was readily converted to glycosyl acceptor 72 and donor 74. Acceptor 72 was obtained in six steps from 68 (38% overall yield) by converting the N3 group to an N-TFA group to form thioglycoside 69, glycosylation to append the fluorous tag, exchange of the 2-OBz group of IdoA with an Fmoc group, and regioselective opening of the Naph acetal of 71. Donor 74 was generated in five steps from 68 (72% overall yield) by exchanging the 2-O-Bz group of IdoA with a Lev group, removal of the Naph acetal, and installation of tert-butyldiphenylsilyl (TBDPS) and benzoyl (Bz) groups at the resulting 6-OH and 4-OH of GlcN, respectively.

Glycosylation of disaccharide donor 74 and acceptor 72 using N-iodosuccinimide (NIS) and silver trifluoromethanesulfonate (AgOTf) at room temperature delivered 65 with exclusively the desired a-stereochemistry (¹J_{C-H}=173.7 Hz) in 80% yield. Importantly, this concise, scalable route furnished the universal building block on a multi-gram scale in only 21 steps, approximately half the number of steps previously reported for HS oligosaccharides of similar complexity.

### Example 4

### Divergent Synthesis of the HS Library

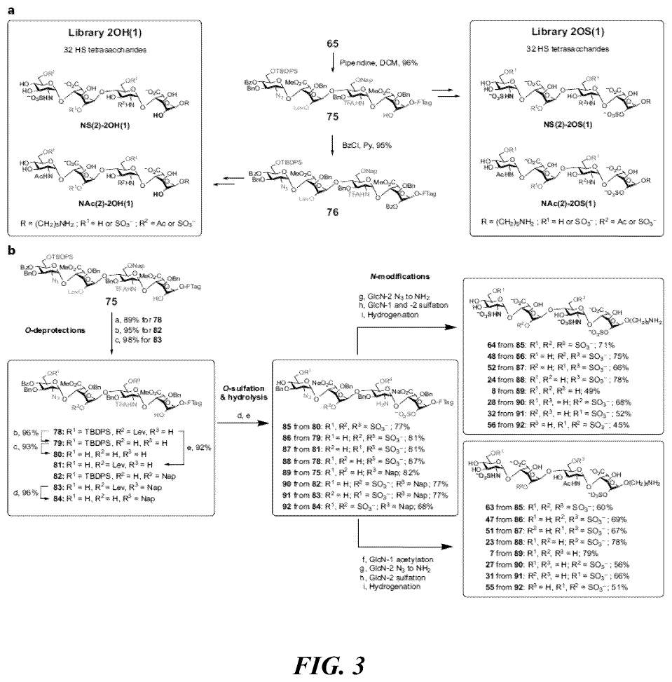

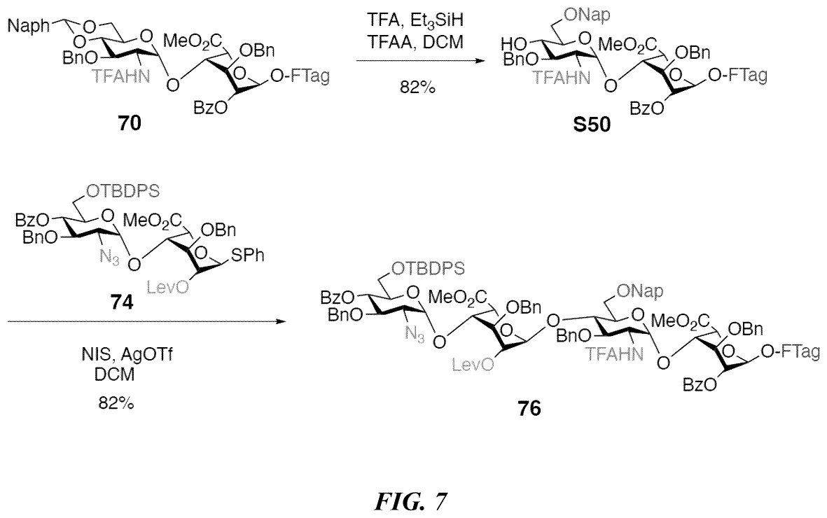

This example describes a divergent strategy for synthesizing the HS library. To access the 64-compound library, 65 was elaborated into two separate pools of 32 compounds via intermediates 75 and 76 (FIG. 3, panel a) and FIG. 7). Intermediate 75, obtained via selective removal of the C2 Fmoc group in 65, served as a precursor for all structures with 2-O-sulfation at the reducing end IdoA-1 (library 2OS(1), 32 compounds). Intermediate 76, obtained via benzoylation of 75, served as a precursor for all structures containing an unsulfated 2-OH at IdoA-1 (Library 2OH(1), 32 compounds).

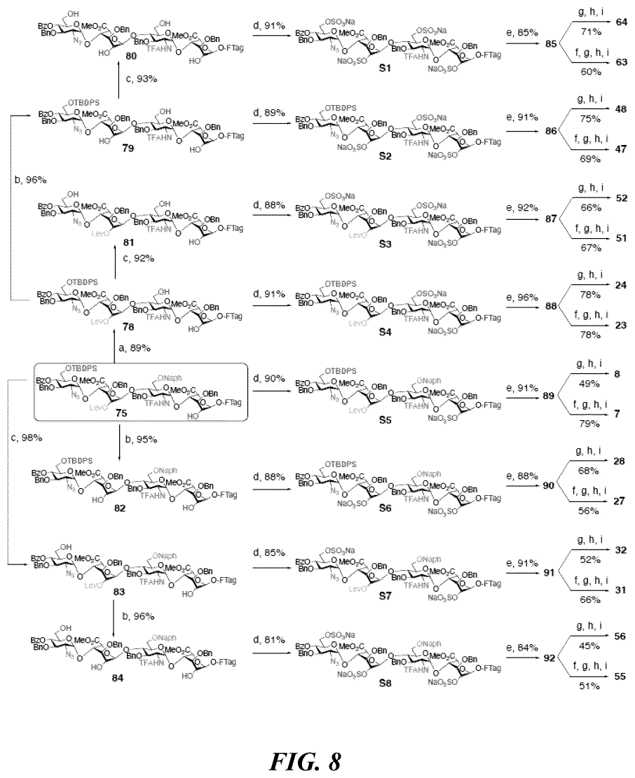

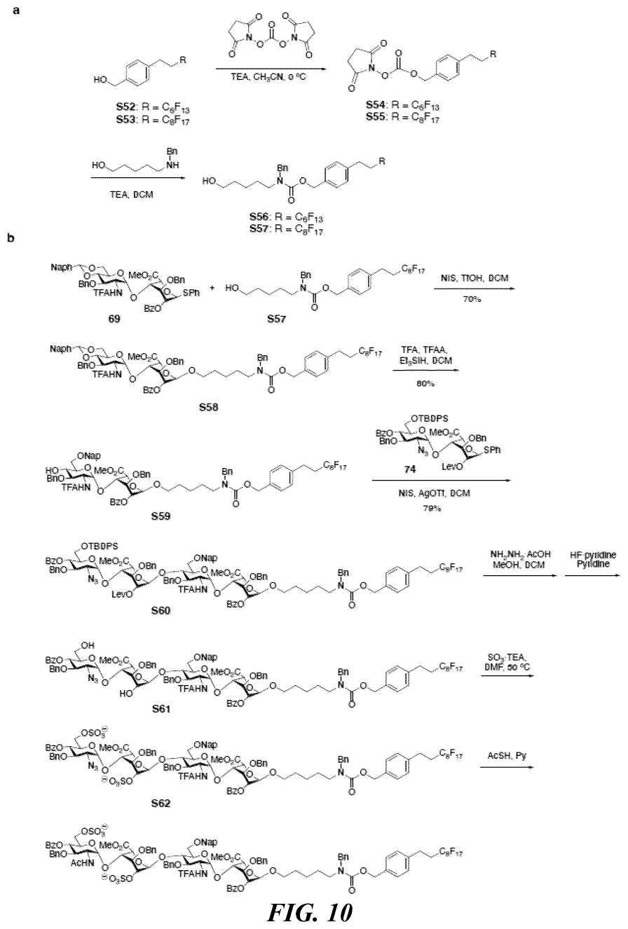

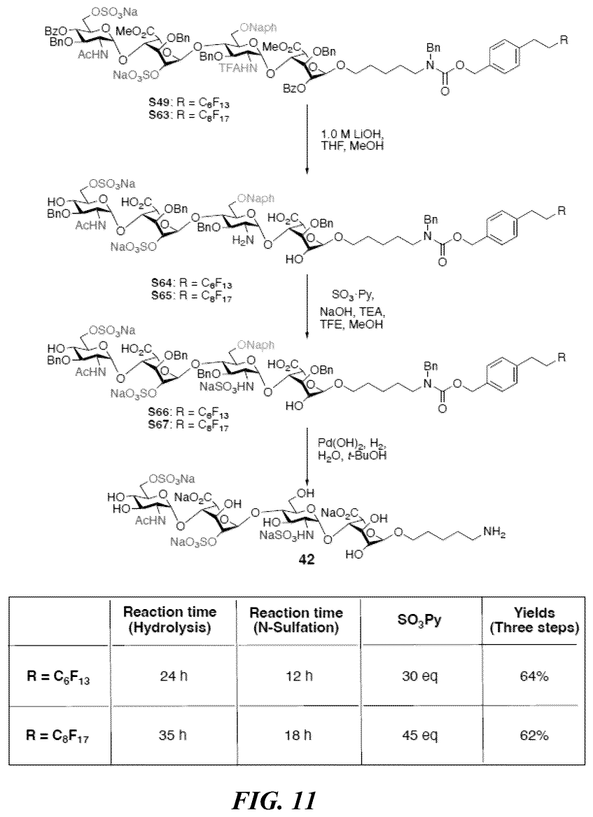

For the 2OS(1) library, two sub-libraries with either N-sulfation (sub-library NS(2)-2OS(1), 16 compounds) or N-acetylation (sub-library NAc(2)-2OS(1), 16 compounds) at the nonreducing end GlcN-2 were obtained from tetrasaccharides 75 and 77, respectively (FIGS. 3, 8, and 9). Treatment of compound 75 with thioacetic acid (AcSH) converted the azide to the corresponding acetamide directly, forming 77 in 83% yield. Chemoselective cleavage of the TBDPS, Lev and/or Nap groups of 75 and 77 was accomplished using hydrogen fluoride in pyridine (HF·Py) or tetra-n-butylammonium fluoride (TBAF), hydrazine acetate (NH2NH2 AcOH) or 2,3-dichloro-5,6-dicyano-1,4-benzoquinone (DDQ), respectively. These various O-deprotections of 75 furnished compounds 78-84 with different combinations of hydroxyl groups unmasked for O-sulfation (FIG. 3, panel b). After O-sulfation using SO₃·Et₃N in DMF, LiOH hydrolysis led to removal of the N-TFA, Bz groups and methyl esters to give compounds 85-92. Under these strongly basic conditions, the TBDPS group was also simultaneously cleaved, which saved one step (TBDPS deprotection) for compounds lacking 6-O-sulfation at GlcN-2 (for example, 75, 78, 79 and 82). Staudinger reduction of the azido group in compounds 85-92, followed by N-sulfation of both amines using SO₃·Py in an aqueous trifluoroethanol (TFE)/MeOH co-solvent mixture, afforded the corresponding hydrogenation-ready tetrasaccharides. However, initial attempts at LiOH hydrolysis and N-sulfation using the fluorous-tagged tetrasaccharides required longer reaction times and more equivalents of SO₃·Py to achieve reaction completion (FIGS. 10-11). After investigating both C₆F₁₃ and C₈F₁₇ tags, it was found that the C₆F₁₃ tag was better suited for GAG synthesis due to its greater solubility in polar solvents. TFE further enhanced the solubility of the fluorous-tagged tetrasaccharides in the aqueous MeOH solvent used for N-sulfation, thereby avoiding aggregation and improving the overall yields.

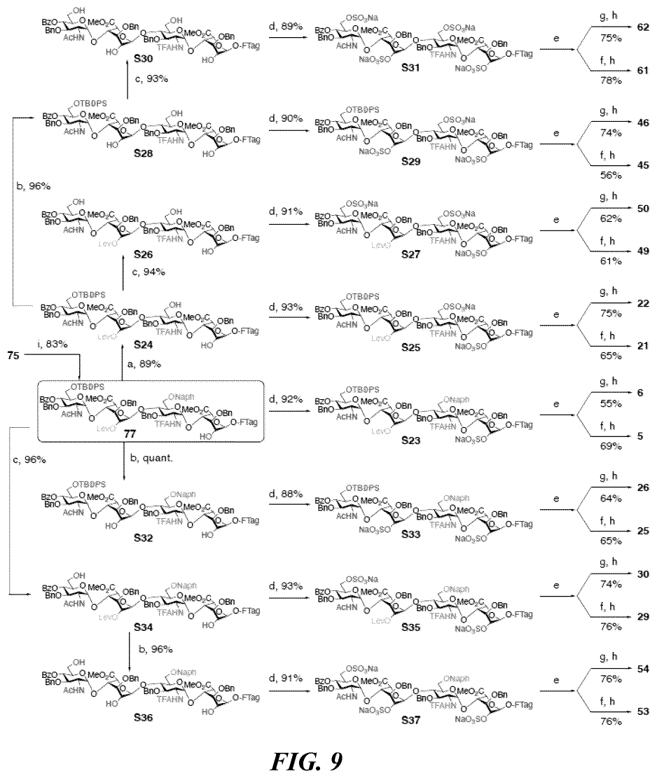

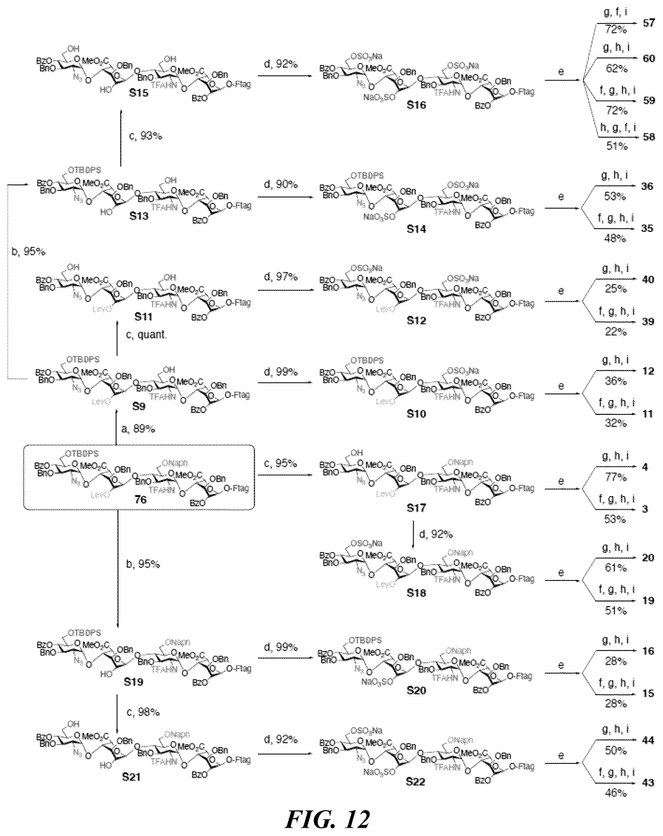

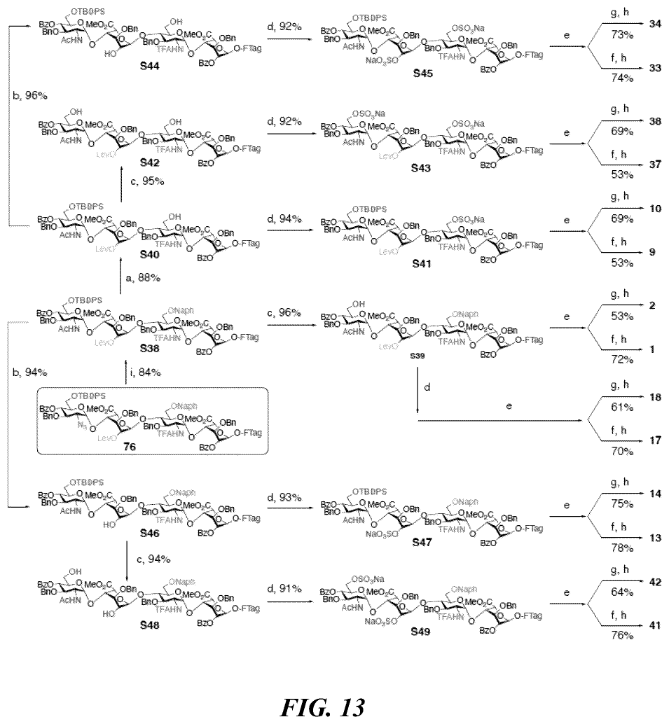

Global hydrogenolysis cleaved all benzyl and Nap groups to generate the eight tetrasaccharides with di-N-sulfation in the NS(2)-2OS(1) sub-library (64, 48, 52, 24, 8, 28, 32 and 56; FIG. 3, panel B). Alternatively, N-acetylation of intermediates 85-92, followed by azide reduction, N-sulfation and hydrogenolysis afforded the other eight mono-N-sulfated tetrasaccharides (63, 47, 51, 23, 7, 27, 31 and 55) in the sub-library. Similar procedures were performed using tetrasaccharide 77 to give the 16-compound NAc(2)-2OS(1) sub-library (FIG. 3, panel a and FIG. 9) and using tetrasaccharide 76 to generate the 32-compound 2OH(1) library (FIGS. 12-13).

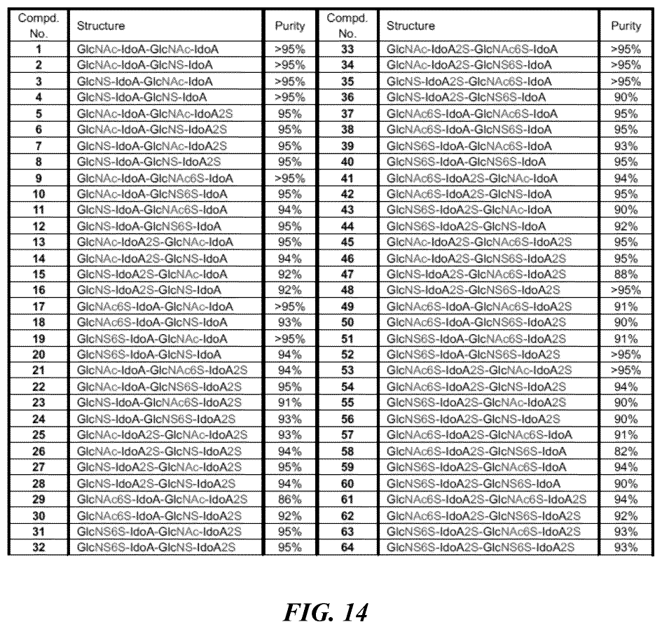

Overall, 64 different HS tetrasaccharides were prepared on a 5-15-mg scale in 86-95% purity from 3.5 g of the universal building block 65 (FIG. 14). This comprehensive library of HS oligosaccharides containing all possible 2-O-, 6-O- and N-sulfation sequences in the tetrasaccharide GlcN-IdoA-GlcN-IdoA represents the most diverse collection of HS tetrasaccharides synthesized so far. Most of the structures are irregular, ‘mixed’ sequences where each disaccharide unit displays a different sulfation motif. Such sequences cannot be obtained through chemoenzymatic synthesis. Our results highlight the power of chemical synthesis to generate both natural and non-natural HS structures with a wide range of regiodefined sulfation patterns.

### Example 5

### Elucidating GAG Structure-Function Relationships

The sulfation preferences of GAG-binding proteins are typically described in general terms (for example, 6-O-sulfation is required), with little information regarding the relative importance of specific modifications at each monosaccharide residue in the sequence. As the library covers the entire chemical space of N-acetyl, N-sulfate, 2-O-sulfate and 6-O-sulfate modifications within the GlcN-IdoA-GlcN-IdoA tetrasaccharide, one can systematically and comprehensively evaluate the impact of modifications at each position and obtain critical, sequence-specific information that was previously unattainable. To assess protein binding to the HS library, glycan microarrays was constructed in which the amine on the reducing end of tetrasaccharides 1-64 was covalently attached to N-hydroxysuccinimide (NHS)-functionalized glass slides. Droplets (<1 nl) of each compound were spotted in nonuplicate at nine different concentrations (0.8-200 μM) using robotic printing technology.

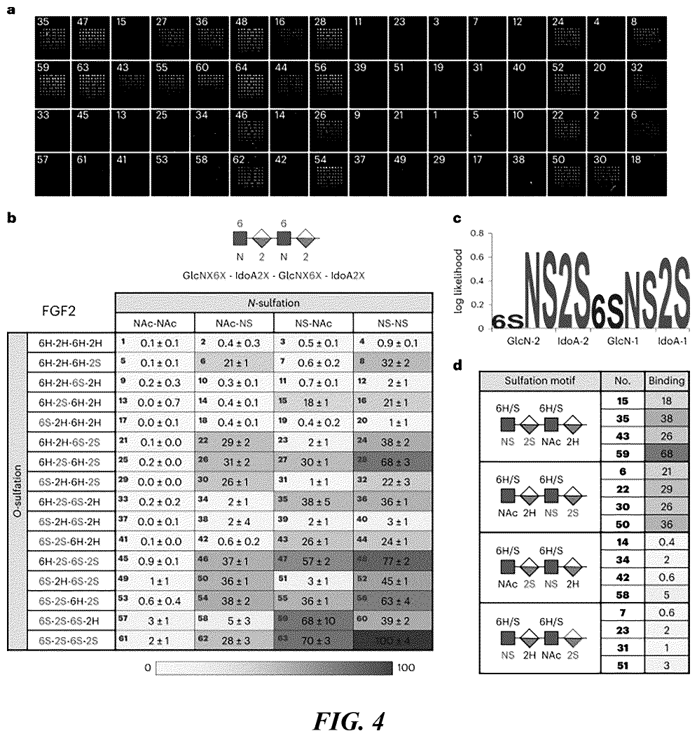

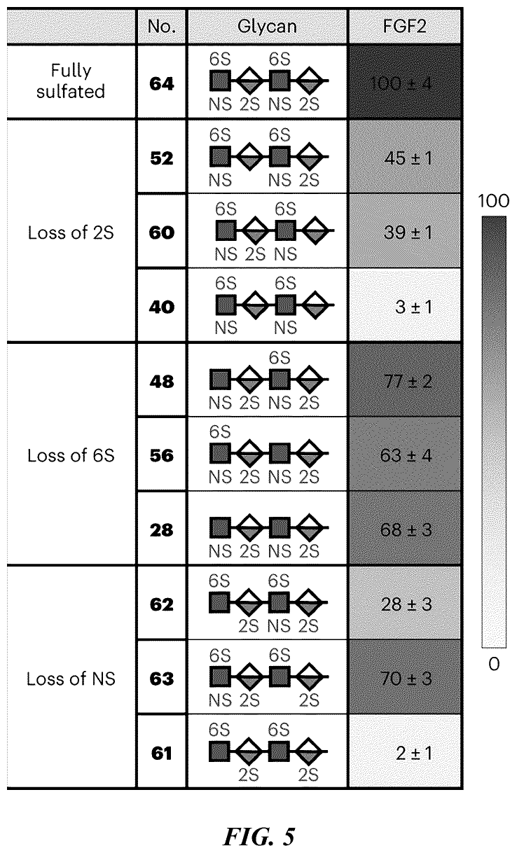

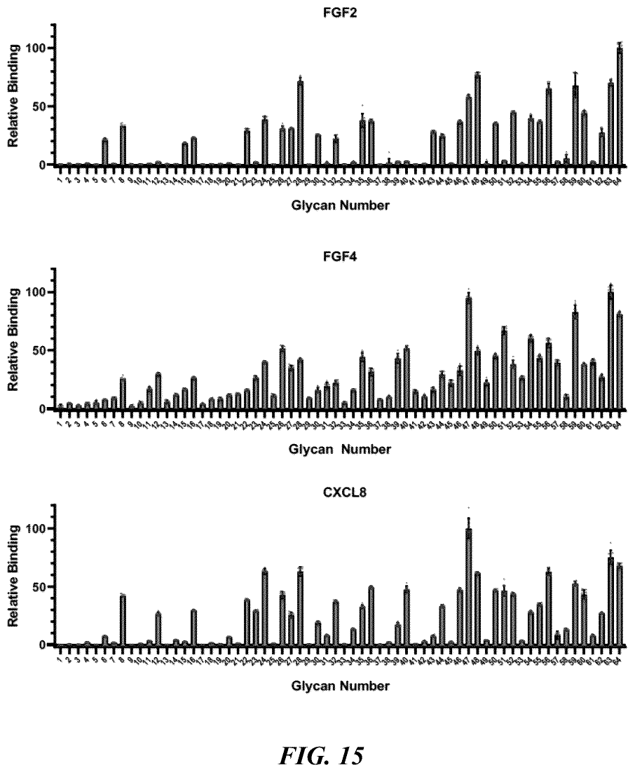

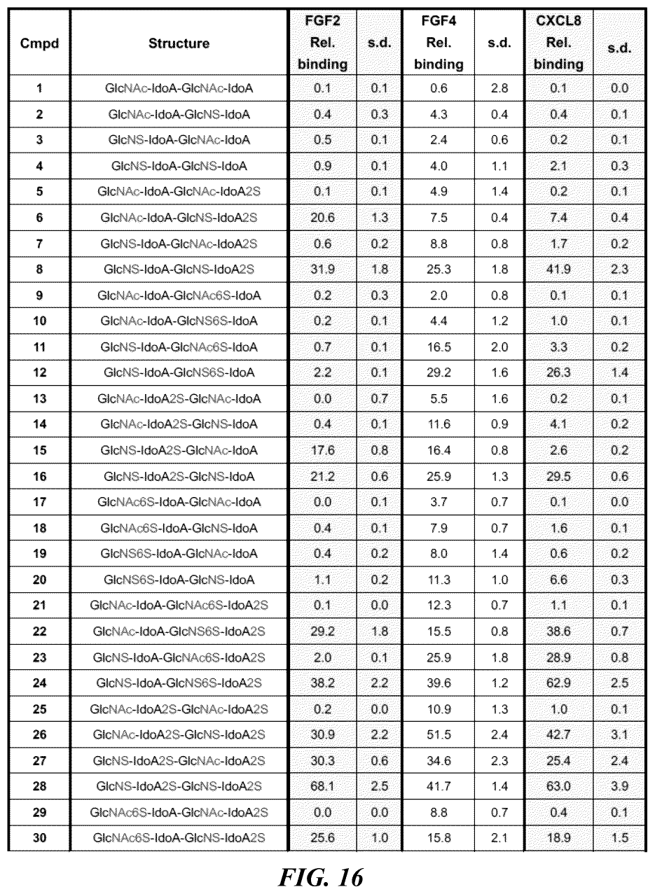

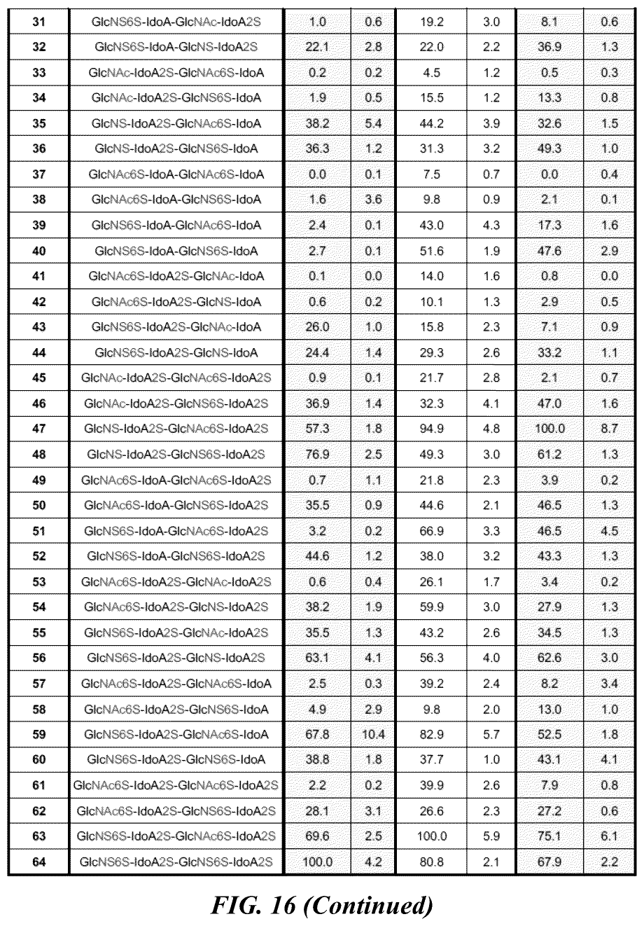

The binding of fibroblast growth factor 2 (FGF2) was first examined, a well-studied HS-binding protein involved in a diverse range of developmental processes and human diseases such as asthma, cancer and cardiovascular diseases. FGF2 binding was detected using an Alexa Fluor 647-labelled anti-His-tag antibody, and relative binding to the 64 tetrasaccharides was represented as both a heatmap (FIG. 4) and bar graph (FIG. 15). Loss of a single 2-O-sulfate group at IdoA-1 or IdoA-2 led to a substantial reduction in FGF2 binding (for example, 64 (100%) versus 60 (39%) and 52 (45%), respectively; FIGS. 4, panel b and 5), whereas loss of both 2-O-sulfate groups abolished binding (40 (3%)). Similar trends were observed across multiple series of compounds (for example, 63 (70%) versus 51(3%) and 39 (2%); 47 (57%) versus 35 (38%), 23 (2%) and 11(0.7%)), indicating that the 2-O-sulfate groups at both IdoA positions are critical for FGF2 recognition. Although both N-sulfate groups were also key contributors (FIG. 5; 64 (100%) versus 61(2%)), N-sulfation was generally more important at GlcN-2 than GlcN-1 (for example, 64 (100%) versus 62 (28%) and 63 (70%), respectively). In contrast, loss of either or both of the 6-O-sulfate groups led to a relatively small reduction in binding (FIG. 5; 64 (100%) versus 48 (77%), 56 (63%) and 28 (68%)), indicating that 6-O-sulfation is not essential for FGF2 recognition.

The above findings highlight an interesting parallel between the molecular recognition of HS GAGs and DNA. DNA-binding transcription factors, like HS-binding proteins, are capable of binding a subset of closely related sequences, and their binding specificities have been characterized using DNA sequence logos. Drawing on these parallels, a general method was developed to quantify and visualize the preferred sulfation sequences of GAG-binding proteins. Briefly, the relative frequencies of each HS modification across the full set of sequences in the library were first determined and weighted by the normalized binding values in the heatmaps. The log ratio of the observed frequency to the expected frequency produced position weight matrices, which were then visualized as ‘sulfation logos’. These logos depict the enrichment (or log-likelihood) of sulfation (S), acetylation (Ac) or no modification (H) at each position on a logarithmic scale within the bound HS sequences, where a positive value indicates a higher probability of occurrence than by chance (FIG. 4, panel c). Sulfation logos thus represent the range of HS modifications bound by a given protein and highlight the extent to which each modification is preferred at each position in the sequence. As GAG-binding proteins often tolerate multiple sequences yet prefer distinct motifs, these logos help to determine and depict the key elements of HS-protein recognition.

In the case of FGF2, the N-sulfate (NS) modification of GlcN-2 is the most overrepresented modification among the bound sequences. The computed sulfation logo reveals that N-sulfation at GlcN-1 is less important overall than at GlcN-2, consistent with the general trends observed. Similarly, 2-O-sulfation (2S) at both IdoA-1 and IdoA-2 is also highly overrepresented. In contrast, 6-O-sulfate (6S) modifications at GlcN-2 and to a lesser extent, GlcN-1, are only slightly more enriched than by chance, indicating that 6-O-sulfation is not critical to FGF2 binding. Together with the heatmap analysis, our results lend further support to a large body of structural and biochemical studies demonstrating the crucial requirements of HS 2-O- and N-sulfation, but not 6-O-sulfation, for FGF2 recognition. Indeed, crystal structures of FGF2 complexed with heparin oligosaccharides show that the 6-O-sulfate groups of heparin point away from the binding site and do not make key contacts with FGF2.

The results also expand an understanding of HS-FGF2 interactions. Interestingly, FGF2 binding was not highly correlated with the overall negative charge of the tetrasaccharides. Tetrasaccharides bearing the same number of sulfate groups displayed a wide range of binding efficiencies. For example, binding to the tetrasulfated compounds 28, 47, 54 and 61 ranged from 2% to 68%, depending on the precise location of the sulfate groups. The pentasulfated tetrasaccharides 60 and 63 also exhibited substantially different interaction with FGF2 (39% and 70%, respectively). These observations reinforce the notion that HS-binding proteins such as FGF2 bind in a sequence-specific manner and that HS-FGF2 interactions are driven by the specific pairing of electrostatic, hydrogen-bonding and van der Waals interactions, rather than cumulative, nonspecific charge effects.

Obtained herein also includes position-resolved, sequence-specific information regarding HS recognition by FGF2 that was previously unknown. Although the importance of the NS and 2S modifications had been shown, it was not known that FGF2 binding required a precise sequence of these modifications. It was found that the NS and 2S modifications must be adjacent to one another within the same GlcN-IdoA disaccharide unit, and the 2-O-sulfated IdoA must be on the reducing end relative to the N-sulfated GlcN (FIG. 4, panel d; GlcNS-2-IdoA2S-2, compounds 15, 35, 43, 59 or GlcNS-1-IdoA2S-1, compounds 6, 22, 30, 50). Separating the NS and 2S modifications (GlcNS-2-IdoA-2-GlcN-1-IdoA2S-1, compounds 7, 23, 31, 51) or changing their reducing end orientation to a nonreducing end orientation (IdoA2S-2-GlcNS-1, compounds 14, 34, 42, 58) abolished FGF2 binding. These unexpected observations suggest that FGF2 binds the HS tetrasaccharides in a preferred orientation, and the FGF2-HS interaction requires a specific sequence of nonreducing-to-reducing end modifications. Thus, our HS libraries enable a deeper understanding of HS-protein interactions, permitting the identification of sequence-specific modifications and specific combinations of modifications critical for interaction. Detailed sequence-specific information has not been previously attainable for HS GAGs, even though it has been vital to the understanding of other major biopolymers such as DNA, RNA and proteins.

### Example 6

### Comparisons Between FGFs

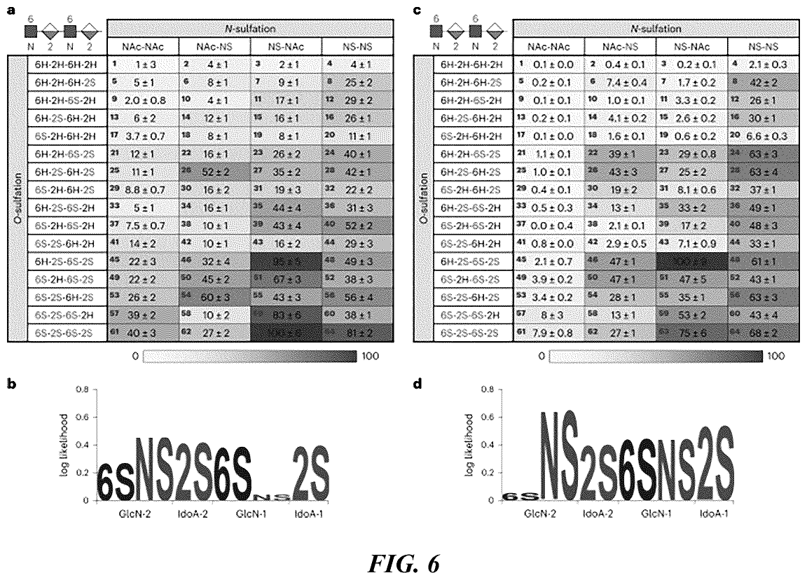

Previous studies have proposed that each FGF family member may recognize a distinct sulfation sequence due to differences in the spatial arrangement of basic residues in their HS-binding sites. The sulfation preferences of another FGF, FGF4, which plays critical roles in biological functions such as angiogenesis and neurogenesis, were chosen for the examination. It was found that the HS-binding heatmaps and sulfation logos for FGF4 and FGF2 are strikingly different (FIG. 4, panels b and c and FIG. 6, panels a and b). In contrast to FGF2, FGF4 showed the highest binding preference for tetrasaccharide 63, which lacks N-sulfation at GlcN-1. Interestingly, loss of the N-sulfate group and N-acetylation at GlcN-1 generally increased FGF4 binding (for example, 64 (81%) versus 63 (100%); 60 (38%) versus 59 (83%); 48 (49%) versus 47 (95%)), and the computed sulfation logo showed no overall preference for N-sulfation or N-acetylation at GlcN-1 in the bound sequences (FIG. 6, panel b). On the other hand, loss of the N-sulfate group at GlcN-2 generally decreased FGF4 binding (for example, 64 (81%) versus 62 (27%); 60(38%) versus 58 (10%); 48 (49%) versus 46 (32%)). Although previous studies have suggested that N-sulfation is important for HS-binding to FGF2 and FGF4, the results presented herein indicate that N-sulfation is not fully required for strong FGF4 binding and reveal the distinct contributions of each N-sulfate group in the tetrasaccharide sequence. Other crucial differences in HS recognition between FGF4 and FGF2 were also identified, including a stronger dependence on 6-O-sulfation for FGF4. Loss of either or both 6-O-sulfate groups substantially reduced FGF4 binding (for example, 63 (100%) versus 55 (43%) and 27 (35%); 64 (81%) versus 56 (56%), 48 (49%) and 28 (42%); 59 (83%) versus 35 (44%), 43 (16%) and 15 (16%)), with 6-O-sulfation generally being more important at GlcN-1 (FIG. 6, panel b), particularly when GlcN-1 lacks N-sulfation (for example, 47 (95%) versus 55 (43%)). Similar to FGF2, loss of 2-O-sulfation at both IdoA-1 and IdoA-2 substantially reduced binding to FGF4 (39 (43%) and 40 (52%)). Overall, the sulfation preferences of FGF4 are remarkably distinct from those of FGF2, suggesting that these FGF family members likely recognize unique HS sequences in vivo. Interestingly, several tetrasaccharides were preferentially recognized by FGF4 but not FGF2 (for example, 51 (67% versus 3%) and 40 (52% versus 3%)), raising the possibility of selective manipulation of FGFs in vivo by endogenous HS expression patterns or exogenous HS mimetics.

### Example 7

### Hs-Chemokine Interactions

HS regulates numerous chemokines important for the immune response, such as CXCL8 (interleukin-8), which is involved in leukocyte migration during bacterial and viral infections, as well as chronic inflammation. In stark contrast to FGF2 and FGF4, the top binding sequence for CXCL8 was compound 47, which lacks 6-O-sulfation at GlcN-2 and N-sulfation at GlcN-1 (FIG. 6, panel c). Contrary to views that greater negative charge on HS leads to higher binding affinity, further increases in charge density were generally detrimental to CXCL8 binding (for example, 47 (100%) versus 63 (75%) and 48 (61%); 24 (63%) versus 52 (43%)), indicating that CXCL8-HS interactions are driven by specific interactions rather than overall negative charge. CXCL8 recognition depended strongly on 2-O-sulfation, as loss of either or both 2S groups substantially decreased binding (47 (100%) versus 23 (29%), 35 (33%) and 11(3.3%); 63 (75%) versus 51(47%), 59 (53%) and 39 (17%)). Although previous studies have reported that 6-O-sulfation and N-sulfation are favourable to CXCL8 binding, our results demonstrate important position- and context-dependent effects for these modifications. For example, 6-O-sulfation at GlcN-1 enhanced CXCL8-HS interactions (27 (25%) versus 47 (100%)), whereas 6-O-sulfation at GlcN-2 was detrimental or neutral to binding (47 (100%) versus 63 (75%); 36 (49%) versus 60 (43%)). Accordingly, the sulfation logo for CXCL8 showed a striking lack of dependence on the 6S modification at GlcN-2 (FIG. 6, panel d). More nuanced effects of 6-O-sulfation were also observed. For example, when GlcN-1 lacked N-sulfation, 6-O-sulfation at GlcN-1 became more important for binding and contributed to the optimal recognition sequence (47 (100%) versus 27 (25%)). On the other hand, when GlcN-1 was N-sulfated, 6-O-sulfation at GlcN-1 had little effect on binding (46 (47%) versus 26 (43%) and 48 (61%) versus 28 (63%)). Notably, N-sulfation was critical for binding, particularly at GlcN-2 (for example, NS-NS column versus NAc-NS column), whereas N-sulfation/N-acetylation at GlcN-1 reduced CXCL8 binding for some highly sulfated sequences (47 (100%) versus 48 (61%); 59 (53%) versus 60 (43%)). Overall, these findings highlight the ability of comprehensive HS oligosaccharide libraries to provide insights into the precise HS modifications required for optimal interaction with CXCL8, a protein that has been challenging to characterize and reported to rely on the domain structure of HS rather than specific modifications.

The disclosure presented herein reveal the different sulfate modification dependencies of CXCL8, FGF4 and FGF2 with residue-specific, sequence resolution that has been previously unattainable. The data suggest that each protein is characterized by a distinct set of HS-binding interactions represented as a heatmap ‘fingerprint’. Furthermore, binding enrichment analyses visualized as sulfation logos highlight the unique features exploited by each protein for molecular recognition of HS.

In summary, a new strategy to prepare large, structurally diverse libraries of HS oligosaccharides was developed. The approach exploits a universal HS building block derived from natural heparin to minimize the number of synthetic transformations and a fluorous tagging method to expedite the purification of charged HS intermediates. This streamlines the late-stage diversification steps and substantially reduces the time and labour required for GAG library synthesis. Overall, this platform greatly accelerates the production of large, comprehensive collections of HS oligosaccharides and represents an important step toward providing broad access to synthetic GAG oligosaccharides on demand.

This approach was then applied to generate a complete library of HS oligosaccharides representing all possible modifications at the most commonly sulfated N-, 2-O- and 6-O-positions in the tetrasaccharide GlcN-IdoA-GlcN-IdoA. This library allowed for detailed, systematic investigations into the structural determinants important for molecular recognition by HS-binding proteins. Methods were also developed to analyze, visualize and compare the HS sequences bound by proteins, and the importance of individual modifications at each position in the oligosaccharide sequence was dissected. The sulfation sequence logos demonstrate that FGFs and chemokines exhibit unique binding preferences across a broad range of HS sequences. Notably, the synthetic approach and sulfation logos presented herein can be readily expanded in the future to incorporate other modifications such as epimerization (that is, GlcA or IdoA) or to represent other GAG classes.

Overall, the streamlined synthesis of comprehensive libraries of defined HS oligosaccharides provides a wide diversity of both natural and non-natural structures for elucidating structure-function relationships. When combined with powerful, high-throughput microarray technologies, these libraries offer an unparalleled view into the sulfation code of GAGs, demonstrating the unique preferences of proteins for specific subsets of closely related sequences and revealing interesting analogies to DNA-binding proteins. Notably, the incorporation of short GAG oligosaccharides into multivalent polymer scaffolds can serve as effective mimetics for GAG polysaccharides. Thus, it is expected that these libraries and the ability to decode GAG structure-function relationships will open new opportunities to target therapeutically important proteins.

### Example 8

### Development of an Automated HS Synthesis Platform

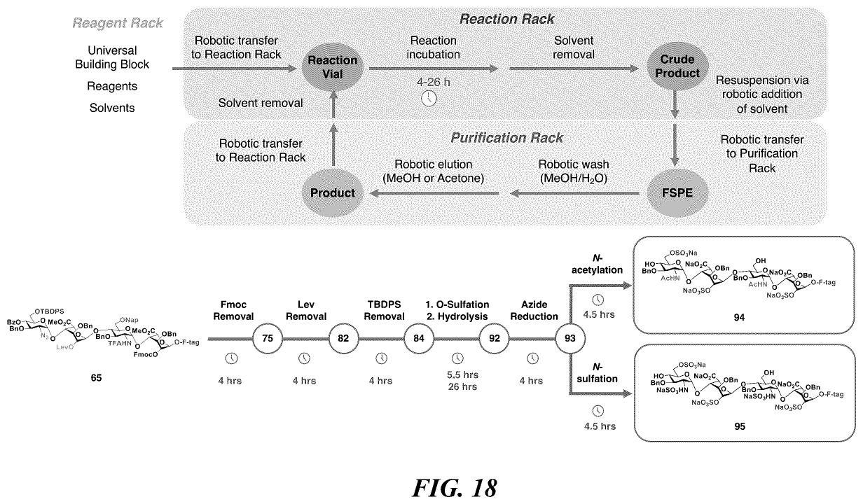

To accelerate access to large collections of compounds, a platform for the automated synthesis of HS libraries was developed. Automating the large number of laborious late-stage modification steps started from the universal F-tagged building block 65. A ChemSpeed Technologies workstation equipped with a robotic arm, 2-channel syringe head, and three separate racks for reagents, reaction vessels, and FSPE cartridges (FIG. 18) was used. To begin the first O-deprotection step, the robotic arm transferred tetrasaccharide 65 in DCM along with other necessary reagents and solvents from the reagent rack into a reaction vessel. The vessel was incubated with shaking at the desired temperature until the reaction was complete. The solvent was then removed under reduced pressure, a suitable solvent was dispensed to resuspend the crude product, and the robotic arm transferred the mixture to a fluorous silica gel cartridge on the purification rack. Automated purification of the product was then performed: the fluorous resin was washed with 1:1 MeOH:H₂O to remove non-fluorous by-products and excess reagents, followed by release of the desired fluorophilic tetrasaccharide product from the resin using MeOH or acetone. The collected eluant was transferred to the reaction rack and dried under reduced pressure to provide the input compound for the next reaction. All steps were performed by the automated system, and the AutoSuite software allowed for easy programming of different late-stage modification workflows in a modular, flexible manner. Although intervention was rarely necessary, the platform could be paused at any stage to allow for efficient reaction monitoring and ensure high product purity.

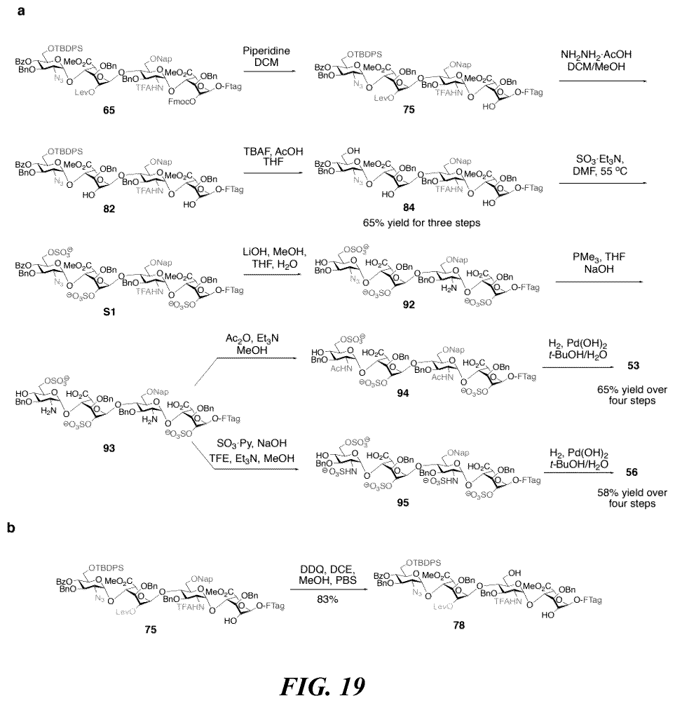

To demonstrate the platform, the synthesis of sulfated tetrasaccharides 53 and 56, representative members of the NAc(1)-2OS(2) and NS(1)-2OS(2) sub-libraries, respectively, that contain a typical number of sulfate groups (FIG. 1, panel a, FIGS. 18-19), was automated. Importantly, these tetrasaccharides enabled all the late-stage modification steps to be verified on the automation platform except for Nap deprotection. The Nap deprotection step was validated separately, with smooth conversion of 75 to 78 demonstrated on the platform in 83% yield (FIG. 19, panel b). To access 53 and 56, tetrasaccharide 65 was first subjected to Fmoc deprotection using the automated platform. After 1 h, the resulting product 75 was purified by automated FSPE, transferred to a new reaction vessel, and subjected to Lev deprotection for 1 h. The resulting Lev-deprotected product was then purified by automated FSPE, transferred back to the reaction rack, and treated with TBAF and acetic acid in THF (as an alternative to the highly corrosive HF) to produce 84 with three unmasked hydroxyl groups. The platform was paused briefly at this stage to ensure the purity and quantify the yield of 84 before subjecting the tetrasaccharide to a round of O-sulfation, hydrolysis, and azide reduction transformations on the automated synthesizer to yield di-amino intermediate 93. At this key branch point between the NAc(1)-2OS(2) and NS(1)-2OS(2) sub-libraries, 93 was split into two aliquots, and N-acetylation or N-sulfation was performed to obtain the desired sulfated tetrasaccharides 94 and 95. The tetrasaccharides were taken directly from the automated platform without further purification and subjected to global hydrogenolysis and size-exclusion chromatography, which delivered the final HS targets 53 (95% purity, 42% overall yield) and 56 (90% purity, 37% overall yield).

Overall, the two sulfated HS tetrasaccharides (94 and 95) were prepared in 8 steps from the universal building block in under 57 hours total, demonstrating the efficiency of this automated approach. For comparison, manual synthesis required approximately 1.5 to 2 weeks. Thus, the platform greatly accelerated the preparation of diverse HS oligosaccharides, enabling their synthesis in a fully automated fashion and in high purity. Notably, this is the first automated platform that has been reported for the synthesis of HS oligosaccharides larger than disaccharides and will facilitate access to large libraries containing a wide diversity of sulfation sequences.

### Example 9

### Materials and Methods

This Example describes general materials and methods used in Examples 10-14 below. Specifically, these examples demonstrate that 4-O-sulfated CS, the dominant sulfation motif on CSPGs in the adult brain, plays a critical role in regulating PNN levels and EI synapse balance in the adult hippocampus.

### Animals

Chst11^{loxP/loxP} and Chst11^{+/-} mice were kindly provided by Prof. Melitta Schachner (Rutgers University). Transgenic mice expressing the Cre recombinase under the control of the human nestin promoter (nestin-Cre^{+/-}) were purchased from Jackson Laboratory. Chst11^{+/-}; nestin-Cre^{+/-} mice, generated by crossing Chst11^{+/-} with nestin-Cre^{+/-} mice, were crossed with Chst11loxP/loxP mice to generate double heterozygous nestin-Cre^{+/-}; Chst11^{loxP/-} mice. These mice were then mated with Chst11^{loxP/loxP} mice to establish animals for the experiments. The resulting nestin-Cre^{-/-}; Chst11^{loxP/-} animals are Ctrl mice, and nestin-Cre^{+/-}; Chst11^{loxP/-} are Chst11cKO mice, as indicated throughout the text. The Chst11cKO mice appeared normal, although they had slightly reduced birth rates (˜2-6 embryos per pregnant mouse). Animals were maintained in the Animal Facility of the California Institute of Technology (Caltech). All animal procedures were performed in accordance with the NIH Guide for the Care and Use of Laboratory Animals and were approved by the Institutional Animal Care and Use Committee of Caltech.

### Antibodies and Chemicals

The following antibodies were used at the indicated dilutions: anti-PSD-95 (1:500, MA1-046) from Thermo Fisher Scientific; mouse anti-gephyrin (1:500, 147 011) from Synaptic Systems; rabbit anti-MAP2 (1:1000, AB5622) and rabbit anti-phospho-CREB (phospho-Ser133, 1:500-1000, 06-519) from Millipore; rabbit anti-CREB (48H2, 1:800, 9197S) from Cell Signaling Technologies; mouse anti-GFP (1:1000, A-11120) from Invitrogen; mouse anti-parvalbumin (1:500, SAB4200545) and rabbit anti-PCP4 (1:200, HPA005792) from Sigma-Aldrich. Fluorescein-labeled Wisteria Floribunda lectin (WFA, 1:300, FL-1351) was purchased from Vector Labs. The mouse anti-CS-E antibody was generated as previously described. Alexa Fluor® (AF) 488 dye-conjugated phalloidin was purchased from Thermo Fisher Scientific (A12379). Chemicals employed in this study include CSA, -C, -E and -D polysaccharides (Seikagaku, Super Special Grade). ChABC (C3667) was purchased from Sigma-Aldrich. The sulfotransferase inhibitor (Inhib) was developed and synthesized by the Hsieh-Wilson laboratory.

### Analysis of Chondroitin Sulfate Disaccharide Composition

Disaccharide analysis was performed as previously described. Cortical regions dissected from mouse brains were homogenized in ice-cold acetone and centrifuged at 4,000×g. The precipitated, air-dried pellets were thoroughly digested with 2 mg/mL of pronase (Sigma-Aldrich) at 55° C. for 24 h. The pronase was then inactivated by heating to 80° C. for 20 min, followed by cooling for 15 min at −20° C. After further digestion with DNase at 37° C. for 1 h, the mixture was centrifuged at 4000 rpm for 15 min. The supernatant was transferred and cooled on ice for 15 min. The proteins were precipitated with 10% (wt/vol) of cold trichloroacetic acid and incubated at 4° C. overnight. The acid-soluble fraction was extracted five times with diethyl ether (1:1, vol/vol). The remaining samples were then left to evaporate in a fume hood for 3 h and subsequently neutralized with 1 M Na₂CO₃. Glycosaminoglycans were precipitated with 5% sodium acetate in 75% ethanol overnight at −20° C. After centrifugation at 4000 rpm for 15 min, the pellet was dissolved in water. An aliquot of the sample was digested with ChABC at 37° C. overnight. The digested glycans were derivatized with aminoacridone (0.1 M solution) and then analyzed by high-performance liquid chromatography (HPLC). Identification and quantification of the disaccharides was achieved by comparison with authentic, unsaturated disaccharide standards derived from natural CS (Seikagaku).

### Neuronal Cultures and Chemical Treatments

Primary hippocampal neuron cultures were prepared as previously described. Briefly, hippocampal tissues were dissected from E15 mouse embryos, and gentle dissociation of neuronal cells was achieved using a papain solution. Isolated neuronal cells were plated on poly-D,Lornithine (PDL)-coated coverslips and cultured for 14-16 days in Neurobasal medium supplemented with B-27 (Gibco™, Thermo Fisher Scientific) and GlutaMAX™ (Invitrogen). Solutions of CS-A, CS-C, CS-D, or CS-E polysaccharides (20 μg/ml final concentration) were added to the culture medium 24 h before fixation of the neurons with 4% (wt/vol) of paraformaldehyde (PFA). ChABC was dissolved in phosphate-buffered saline (PBS) containing 0.01% bovine serum albumin (BSA), according to the manufacturer's instructions. ChABC was then added to the culture medium at 0.05 U/ml, 2, 8 or 24 h before fixation of the neurons. PBS was used as a control. The sulfotransferase inhibitor (Inhib) was dissolved in DMSO before applying to the culture medium of neurons at a final concentration of 10 μM for 24 h. DMSO was used as a control.

### Immunohistochemistry

After 14-16 days in vitro (DIV), cultured hippocampal neurons were fixed with 4% (wt/vol) PFA solution for 15 min. Neurons were then incubated with primary antibodies diluted in blocking buffer (5% vol/vol goat serum, 0.4% Triton X-100 in PBS, pH 7.4), overnight at 4° C. After three washes with PBS, the neurons were incubated with AlexaFluor (AF)-conjugated secondary antibodies in blocking buffer at 22-25° C. for 1.5 h and washed three times with PBS. Immunohistochemistry of brain sections was performed as previously described (6). Briefly, mice were transcardially perfused with PBS followed by 4% PFA. Brains were dissected and post-fixed in 4% PFA at 4° C. overnight. Brains were then transferred to 15% sucrose, then 30% sucrose, in 0.1 M PBS (pH 7.4) each overnight and embedded in optimal cutting temperature (OCT) compound (SAKURA). Coronal sections (30 μm) were cut using a Leica CM1850 cryostat and stored in 30% ethylene glycol and 15% sucrose in PBS at −20° C. Slices were stained with antibodies diluted in blocking buffer (5% vol/vol goat serum, 0.1% Triton X-100 in PBS, pH 7.4), overnight at 4° C. Coverslips and slices were mounted with VECTASHIELD Mounting Medium (Vector Labs).

### Viruses

AAV5-CamKIIα-mCherry and AAV5-CaMKIIa-mCherry-Cre were obtained from the Vector Core at the University of North Carolina at Chapel Hill. Vesicular stomatitis virus G(VSV-G) pseudotyped lentivirus expressing GFP was packaged in the Hsieh-Wilson laboratory.

### Stereotaxic Surgery

Viral delivery was performed as previously described. The VSV-G pseudotyped lentivirus expressing GFP was injected into the hippocampal CA1/CA2 regions (anteroposterior, −2.0 mm; mediolateral, ±1.7 mm; dorsoventral, −1.5 mm; all coordinates relative to bregma) or CA3 region (anteroposterior, −1.7 mm; mediolateral, ±1.9 mm; dorsoventral, −1.9 mm; all coordinates relative to bregma). A 3-μl aliquot of lentivirus was injected bilaterally using a stereotaxic injector (Kopf Model 900). Four weeks after injection, brain sections from the lentivirus-infected mice were incubated with a GFP antibody overnight at 4° C., followed by incubation with an Alexa Fluor® (AF) 488 dye-conjugated secondary antibody for 2 h at room temperature (RT). 4′,6-Diamidino-2-phenylindole (DAPI, Thermo Fisher, 1:3000) was added to the PBS wash buffer to stain for 10 min. Alternatively, 200 nl of either AAV5-CamKIIα-mCherry or AAV5-CaMKIIα-mCherry-Cre was injected bilaterally into the hippocampal CA2 regions of Chst11^{loxp/loxp} mice (anteroposterior, −1.6 mm; mediolateral, ±1.6 mm; dorsoventral, −1.7 mm; all coordinates relative to bregma). Mice were subsequently subjected to a range of behavioral tests three weeks after surgery. ChABC injections were performed similarly. Briefly, 1 μl of ChABC or penicillinase (10 U/ml) was injected bilaterally into the hippocampal CA2 region. Injected mice were either euthanized for immunohistochemistry or subjected to behavioral tests, 2 weeks after surgery.

### Image Acquisition and Quantification

All stained neuron and brain sections were imaged using a Zeiss 700 confocal microscope, with the investigator blind to the treatment or genotype. For PNN studies in cultured hippocampal neurons, at least five regions under the visual field were randomly imaged to represent the culture condition. Each image was captured throughout the entire depth of the region by z-stacking. Quantification of the cell numbers labeled by specific markers, such as WFA, was done manually. Quantification of the size and density of PSD-95 and gephyrin, as well as the density of p-CREB and total CREB, was performed using Image-J, as previously described. Briefly, for puncta analysis, three dendrites of each neuron were selected and cropped from the original image for analysis. The cropped images were color separated, and the channel of interest was analyzed after setting a fixed threshold for all the conditions. The readout was generated with the following two parameters: a) total count, with each unit corresponding to one puncta; and b) area, indicating the size of the puncta. The intensity analysis of p-CREB and total CREB was performed similarly, and the total intensity of each single neuron was obtained after setting a threshold for the image. For dendritic spine analyses, we considered four different spine shapes, including filopodia, stubby, thin, and mushroom. Mushroom-shaped spines are considered mature and form stabilized synaptic connections (8). The density of mature spines was therefore calculated by dividing the number of dendritic spines with mushroom-shaped heads by the length of the dendrite. The head width of each protrusion and the length of each dendrite were measured, as well as the total number of protrusions on each dendrite.

### Electrophysiology

Hippocampal slices were obtained from 5-7 week old Ctrl or Chst11cKO mice. After decapitation, mouse brains were extracted, trimmed, and 400-μm transverse hippocampal slices were cut on a vibratome (VT-1000s, Leica) in ice-cold sucrose-artificial cerebrospinal fluid (aCSF) solution (in mM: 213 sucrose, 2.5 KCl, 1.2 NaH₂PO₄, 25 NaHCO3, 10 glucose, 7 MgSO₄, 1 CaCl₂, pH 7.3). Slices were then incubated in normal aCSF (in mM: 124 NaCl, 2.5 KCl, 1.2 NaH₂PO₄, 24 NaHCO₃, 25 glucose, 1 MgSO₄, 2 CaCl₂), bubbled with 95% O2/5% CO₂) at 34.5° C. for 30 min, and then kept at RT until use. Whole-cell voltage clamp recordings were performed on pyramidal neurons in the CA2, identified based on their anatomical features. Electrical signals were filtered at 3 kHz with an Axon MultiClamp 700B (Molecular Devices) and collected at 20 kHz with an Axon Digidata 1550A (Molecular Devices). For miniature EPSCs recordings, pipette solution contained (in mM:145 Cs(CH₃)SO₃, 2 NaCl, 10 HEPES, 0.2 EGTA, 5 QX-314 bromide, 4 Mg-ATP, 0.3 Na-GTP, pH 7.25). Tetrodotoxin (TTX, 0.5 μM) and picrotoxin (PTX, 100 μM) were included in the aCSF. For miniature IPSCs recordings, pipette solution contained (in mM: 145 CsCl, 2 NaCl, 10 HEPES, 0.2 EGTA, 5 QX-314 bromide, 4 Mg-ATP, 0.3 Na-GTP, pH 7.25). TTX (0.5 μM), 6-cyano-7-nitroquinoxaline-2,3-dione (CNQX, 10 μM) and DL-2-amino-5-phosphonopentanoic acid (APV, 25 μM) were included in the aCSF.

### Two-Trial Social Memory Test

Two-trial social memory experiments were performed as previously described (9). The subject mice were 2-3 months old male mice, and the stimulus mice were 4-6 weeks old male mice. At the beginning of the test, each subject mouse was singly housed in a cage similar to its home cage for 30 min. The lid of the cage was removed to allow filming of social activities. Each subject mouse was left in the cage to habituate for 30 min under dim lighting conditions. After habituation, a group-housed stimulus mouse was placed in each cage for the first interaction of 2 min. The stimulus mouse was removed after the first trial, then re-introduced to the same subject mice after 30 min. The second trial lasted another 2 min. All mice were then returned to their home cages and group housed. The test trials were videotaped and subsequently analyzed for the total interaction time between each subject mouse and the stimulus mouse.

### Anxiety-Related Behavioral Tests

Adult mice of both genders, aged 2-3 months, were used for all anxiety-related tests. The OFT were performed as previously described. Briefly, each mouse was placed in the lower left corner of a 50×50 cm white box, and activity was recorded for 10 min by a ceiling-mounted video camera. The total distance traveled, and center entries were scored using EthoVision software. After each behavioral test, mice were returned immediately to their home cage. The elevated plus maze (EPM) was performed as previously described. Briefly, mice were exposed for 5 min to an elevated plus maze (elevated 35 cm, arms 29×7.7 cm, closed arm walls 16.5 cm, center piece 7.7×7.7 cm, low light condition). Each mouse was placed in the central area of the maze with open access to any arm. The number of arm entries and the amount of time spent in the open and closed arms were recorded for subsequent scoring, using video tracking systems. The elevated plus maze was cleaned with water and disinfectant (NPD®) between mice. For the light-dark box test (LDB), each mouse was initially placed in the dark compartment of the light-dark box (lit chamber 30×25.5 cm, dark chamber 15×25.5 cm, height 30 cm, opening 5×5 cm, bright light condition). The movements of the mouse inside the box were recorded for 10 min to assess bright space anxiety. Before and after each experimental trial, the surfaces of the training and testing boxes are cleaned with water and disinfectant (NPD®).

### Statistical Analyses

All experimental and data analysis in this work were conducted blinded, including the immunohistochemistry, electrophysiology and behavioral tests. The number of replicates (n) is indicated in each figure legend and all experiments were performed at least in triplicate independently unless otherwise indicated. Data are expressed as the mean±SEM. The unpaired, two-sided Student's t test or one-way and two-way analysis of variance (ANOVA) was used followed by Tukey's multiple comparisons to evaluate the significance of differences between two experimental conditions and three or more experimental conditions, respectively. Non-normally distributed data were analyzed by Wilcoxon rank-sum test. The Kolmogorov-Smirnov test was used to study cumulative distribution functions. The level of significance was set at *P<0.05, **P<0.01, ***P<0.001, ****P<0.0001.

### Example 10

### Chst11cKO Mice Lacking 4-O-Sulfation Show Increased WFA⁺ PNN Densities in the CA2 Hippocampus

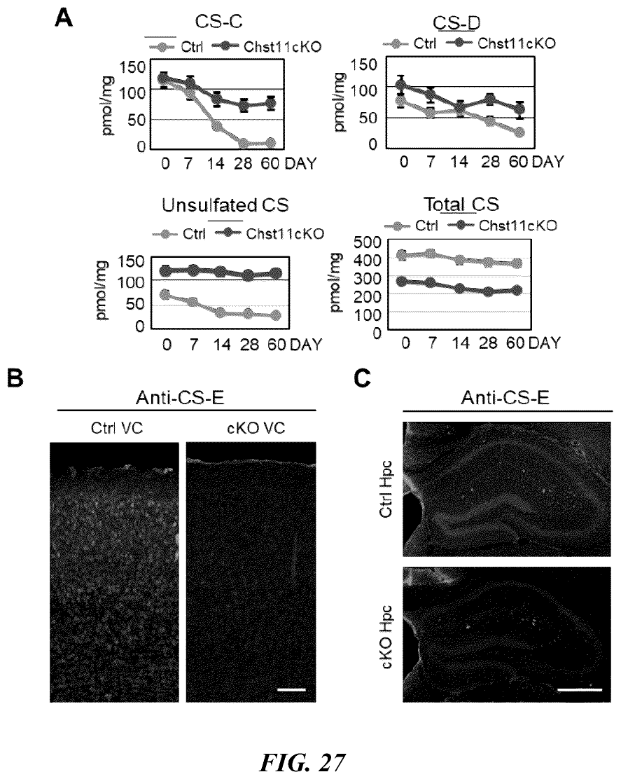

The sulfotransferase Chst11 selectively transfers a sulfate group from 3′-phosphoadenosine 5′-phosphosulfate to the 4 O-position of GalNAc residues in CS polysaccharides. Genetic disruption of Chst11 in mice led to severe chondrodysplasia and neonatal lethality, demonstrating essential roles for 4-O-sulfation in embryonic development. To study the roles of CS 4-O-sulfation in the adult brain, we generated a brain-specific Chst11 conditional knockout (cKO) mouse line by crossing Chst11-floxed mice with nestin-Cre transgenic mice. The nestin-Cre transgene restricts Chst11 deletion to neural precursor cells starting at embryonic day 10.5 (E10.5), circumventing complications associated with constitutive Chst11 ablation during prenatal development. To validate the gene deletion efficiency, we analyzed the sulfation patterns of CS in the cortex of Chst11^{lx/-}; nestin-Cre+ (Chst11cKO) mice and Chst11^{lx/-}; nestin-Cre⁻ (Ctrl) mice as a control. CS chains were isolated from Chst11cKO and Ctrl mouse cortices at different ages (P0, P7, P14, P28, and P60), digested with ChABC, and the resulting disaccharides were quantified by high-performance liquid chromatography. No 4-O-sulfated CS-A and CS-E disaccharide units were detected in Chst11cKO brains, indicating disruption of the 4-O-sulfation pathway (FIG. 21, panels A and B). Loss of chondroitin 4-O-sulfation was accompanied by an increase in unsulfated CS levels and a decrease in overall CS levels in the brain (FIG. 27, panel A), consistent with reports that 4-O-sulfation facilitates CS chain elongation. In contrast, no loss of 6-O-sulfation was observed: CS-D levels remained similar, and CS-C levels increased slightly in Chst11cKO mice compared to Ctrl mice after P14, presumably due to compensatory effects (FIG. 27, panel A). Disruption of the 4-O-sulfation pathway in Chst11cKO mice was further confirmed by immunohistochemical analysis. A marked reduction in CS-E immunostaining was observed in the cortex and hippocampus of Chst11cKO mice relative to Ctrl littermates (FIG. 27, panel B and C). Collectively, these results indicate efficient deletion of the 4-O-sulfation pathway in Chst11cKO mice.

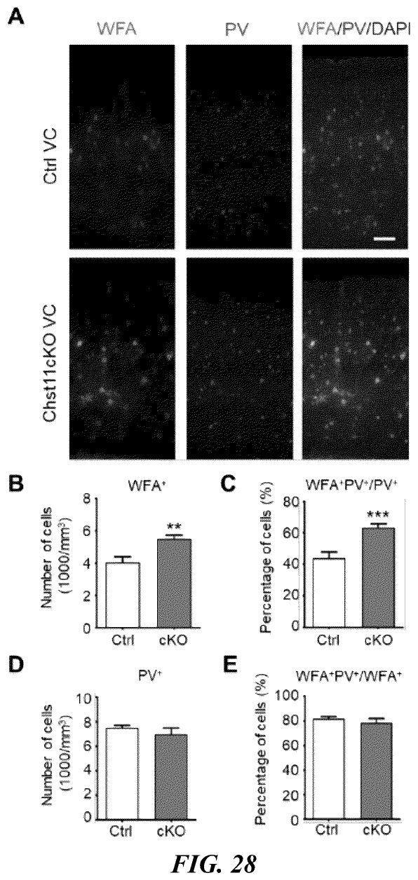

Next, the PNN levels in the VC and hippocampus of adult Chst11cKO and Ctrl mice were examined using the well-established marker, Wisteria floribunda agglutinin (WFA). Consistent with previous observations, WFA⁺ PNNs exhibited region-specific expression patterns. In the VC, an increase in PNN-enwrapped (WFA⁺) neurons was observed in adult Chst11cKO mice compared to Ctrl mice (36%±11%), and this increase occurred primarily in PNNs surrounding the PV⁺ population (FIG. 28, panels A-C). No change was observed in the total number of PV⁺ neurons or its percentage within the WFA⁺ population (FIG. 28, panels D and E). Notably, the greatest change in WFA⁺ PNN-enwrapped neurons was observed in the area CA2 of the hippocampus. Both Chst11cKO and Ctrl adult mice showed high PNN levels in the CA2 region, whereas the CA1 and CA3 areas had fewer WFA⁺ PNNs (FIG. 21, panel C). A striking increase in the number of WFA⁺ PNNs was observed in the CA2 region (109%±15%) and, to a lesser extent, in the CA3 region (55%±13%) of Chst11cKO compared to Ctrl mice (FIG. 21, panel C). Given the substantial increase in WFA⁺ PNNs detected in the CA2 and the limited understanding of CS function in this region, we focused our studies hereafter on the CA2 hippocampus.

As PNNs condense around PV⁺ inhibitory neurons in many brain regions, whether the WFA⁺ PNNs in the CA2 surround PV⁺ neurons was investigated. Adult hippocampal sections were costained for PV and the CA2 marker Purkinje cell protein 4 (PCP4). The PCP4⁺ neurons in the CA2 region of both Chst11cKO and Ctrl mice contained only a small percentage of PV⁺ neurons (8%±2%; FIG. 21, panel D, Upper Right). The majority of the PCP4⁺ neurons in Chst11cKO mice were enwrapped by WFA⁺ PNNs (95%±2%; FIG. 21, panel D, Lower Right), whereas fewer PCP4′ neurons in Ctrl mice were surrounded by WFA⁺ PNNs (33%±8%). These findings are consistent with previous reports that PNN-enwrapped excitatory neurons are prevalent in the CA2 hippocampus and indicate that CS 4-O-sulfation regulates the density of PNNs surrounding excitatory CA2 neurons.

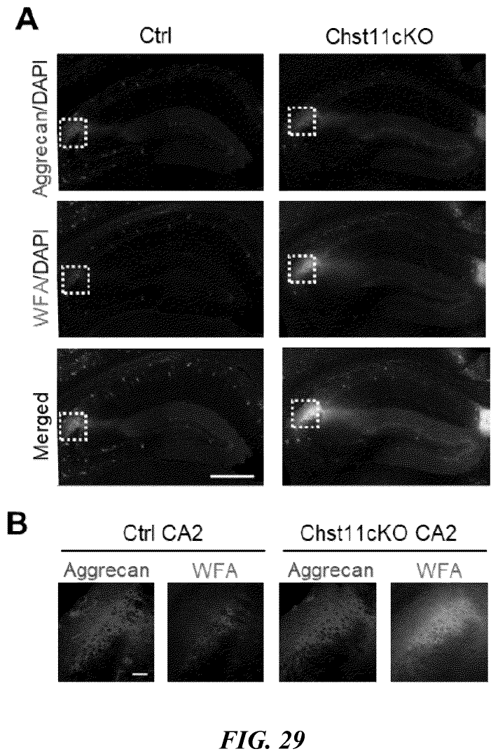

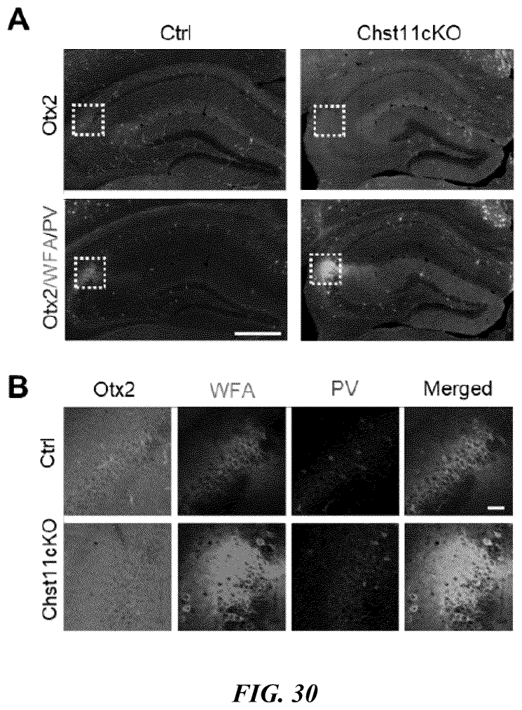

Next, potential mechanisms by which loss of 4-O-sulfation could alter PNNs in the adult hippocampus were examined. Previous studies have shown that increasing CS 6-O-sulfation by overexpression of C6ST-1 in transgenic mice decreases accumulation of the CSPG aggrecan in PNNs, possibly by accelerating its proteolysis by a disintegrin and metalloproteinase with thrombospondin motifs (ADAMTS) protease. However, immunohistochemical analysis of Chst11cKO and Ctrl mice revealed high, comparable expression levels of aggrecan in the CA2 region (FIG. 29). The homeobox protein Otx2 localizes to PNNs by binding to the CS-E motif on CSPGs and regulates PNN assembly and maintenance, as well as PV maturation in the VC. Comparable but low levels of Otx2 expression were observed in the hippocampus of both Chst11cKO and Ctrl mice (FIG. 30). Together, these findings suggest that CS 4-O-sulfation likely regulates PNN densities in the CA2 hippocampus via mechanisms independent of both aggrecan stability and Otx2.

### Example 11

### CS 4-O-Sulfation Regulates Dendritic Spine Maturation and Excitatory-to-Inhibitory Synaptic Ratios

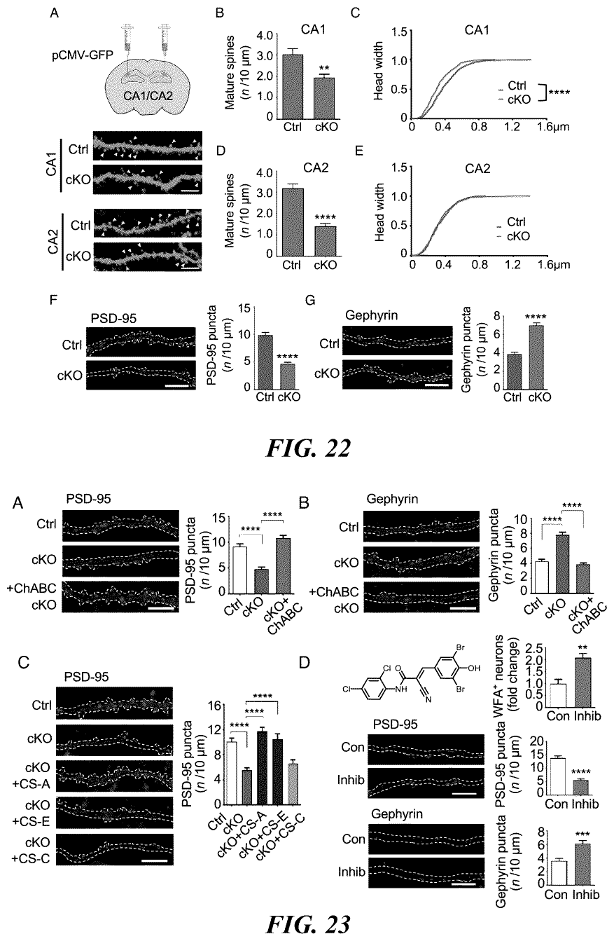

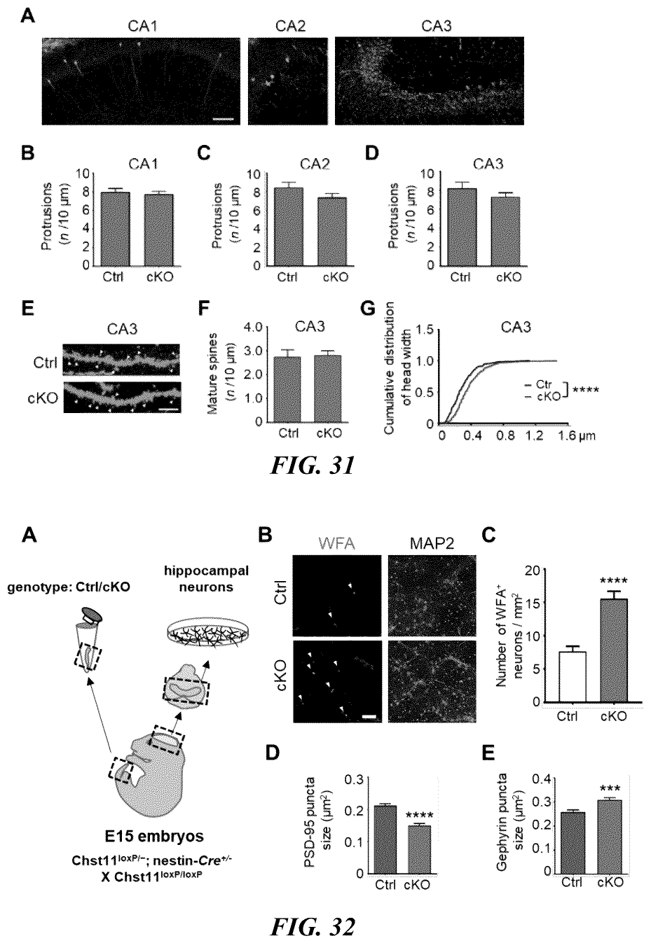

PNNs influence a broad range of synaptic functions, thereby affecting dendritic spine morphology, synapse stability, and both excitatory and inhibitory neurotransmission. To examine the effects of CS 4-O-sulfation on dendritic spine morphology, GFP-expressing lentivirus was injected into the hippocampal CA1, CA2, or CA3 regions of Chst11cKO or Ctrl mice to sparsely label dendrites and dendritic spines (FIG. 22, panel A and FIG. 31, panel A). Notably, the density of mature spines on both CA1 and CA2 neurons was significantly decreased in Chst11cKO mice compared to Ctrl mice (FIG. 22, panels B and D), whereas the density of dendritic protrusions was unchanged (FIG. 31, panels B and C). Furthermore, the head width of dendritic spines was significantly reduced on CA1 but not CA2 neurons in Chst11cKO compared to Ctrl mice (FIG. 22, panels C and E). Interestingly, CA3 neurons in Chst11cKO mice had an enlarged average spine head width, but no difference in mature spine or protrusion density, compared to CA3 neurons in Ctrl mice (FIG. 31, panels D-G). As CA2 pyramidal cells are known to gate hippocampal CA3 excitability via feedforward inhibition, it is conceivable that the excess PNNs surrounding CA2 neurons in Chst11cKO mice may inhibit CA2 output and thus enhance synaptic transmission in the CA3 area, leading to enlarged dendritic spine heads on CA3 neurons. Taken together, these findings indicate that CS 4-O-sulfation is required for the proper maturation of dendritic spines in the hippocampus.

Dendritic spines harbor most excitatory synapses, and the excitatory-to-inhibitory (E/i) synaptic ratio can serve as a good indicator of synaptic balance. Thus, we investigated whether loss of CS 4-O-sulfation altered the development of excitatory and inhibitory synapses. Chst11cKO mice were crossed with Chst11^{loxp/loxP} mice, and hippocampal neurons from each mouse embryo were cultured separately and genotyped (FIG. 32, panel A). We observed a large increase in WFA⁺ PNNs surrounding hippocampal neurons cultured from Chst11cKO mice compared to Ctrl mice (FIG. 32, panels B and C), similar to observations in hippocampal brain sections (FIG. 21, panel C). Hippocampal neurons from Chst11cKO mice also had fewer mature excitatory synapses relative to hippocampal neurons from Ctrl mice, as measured by a reduction in the density and size of PSD-95-expressing puncta (FIG. 22, panel F and FIG. 32, panel D). A concomitant increase in inhibitory synapses was observed, as quantified from the density and size of gephyrin-expressing puncta (FIG. 22, panel G and FIG. 32, panel E). Thus, loss of CS 4-O-sulfation in Chst11cKO hippocampal neurons attenuates excitatory synapse development and promotes the formation of inhibitory synapses.

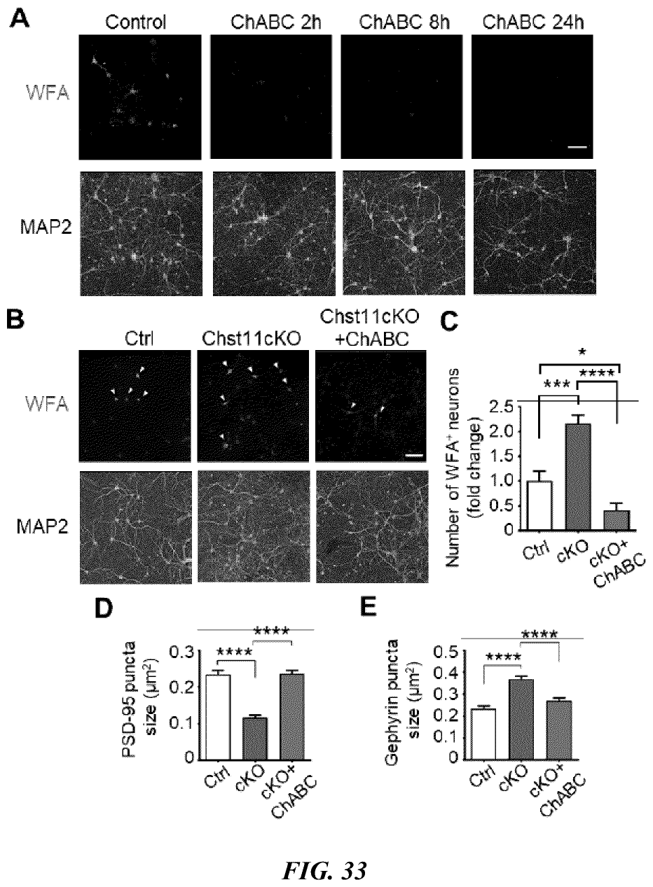

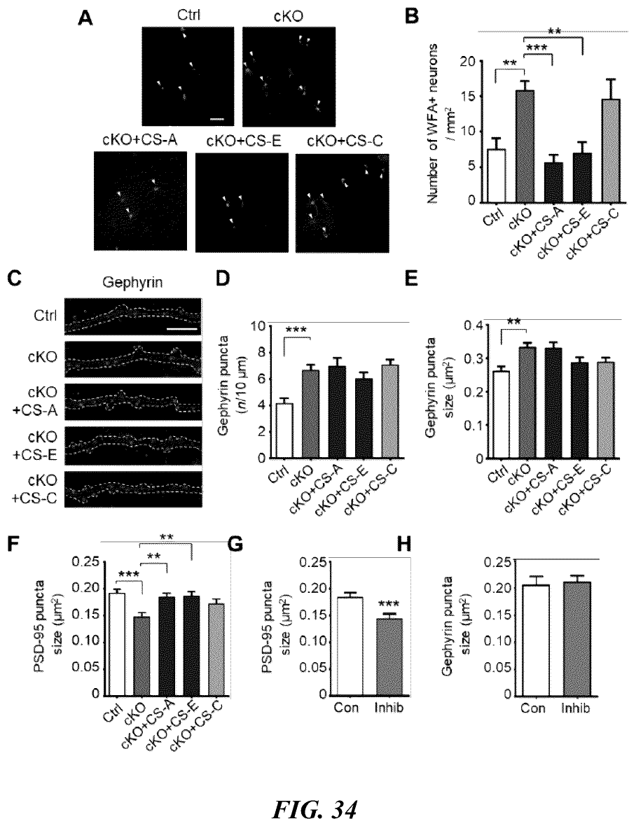

Experiments were further carried out next to investigate whether these synaptic defects were the result of 1) the higher PNN densities observed in the hippocampus of Chst11cKO mice and/or 2) loss of the CS-A and CS-E sulfation motifs in PNNs. To reduce PNN density, hippocampal neurons from Chst11cKO or Ctrl mice were treated with ChABC. Efficient removal of PNNs on cultured hippocampal neurons was achieved after 2 h of ChABC treatment (FIG. 33, panels A-C). Notably, ChABC treatment fully restored the density and size of both PSD-95 and gephyrin puncta to Ctrl levels (FIG. 23, panels A and B and FIG. 33, panels D and E). To investigate the effects of CS sulfation on synapses, we treated Chst11cKO neurons with exogenous, natural polysaccharides enriched in specific sulfation motifs. Treatment of Chst11cKO neurons with either CS-A- or CS-E-enriched polysaccharides restored the number of WFA⁻ PNN-enwrapped neurons (FIG. 34, panels A and B), as well as the density and size of excitatory synapses (FIG. 23, panel C and FIG. 34, panel F), back to Ctrl levels. In contrast, CS-C polysaccharides enriched in 6-O-sulfation had no effect on PNN levels or excitatory synapses (FIG. 23, panel C and FIG. 34, panels B and F), demonstrating the importance of the sulfation pattern. Interestingly, none of the CS polysaccharides rescued the inhibitory synaptic defects of Chst11cKO hippocampal neurons (FIG. 34, panels C-E), suggesting that CSPGs and PNNs regulate excitatory and inhibitory synapses via distinct mechanisms. Importantly, the effects of Chs11 genetic deletion on PNN density and E/I synaptic ratio were recapitulated by chemical inhibition of CS 4-O-sulfation using a small-molecule sulfotransferase inhibitor (FIG. 23, panel D and FIG. 34, panels G and H). Thus, chemical, enzymatic, or genetic manipulation of the 4-O-sulfation motifs on CSPGs can modulate PNN densities surrounding hippocampal neurons and the balance of excitatory and inhibitory synapses.

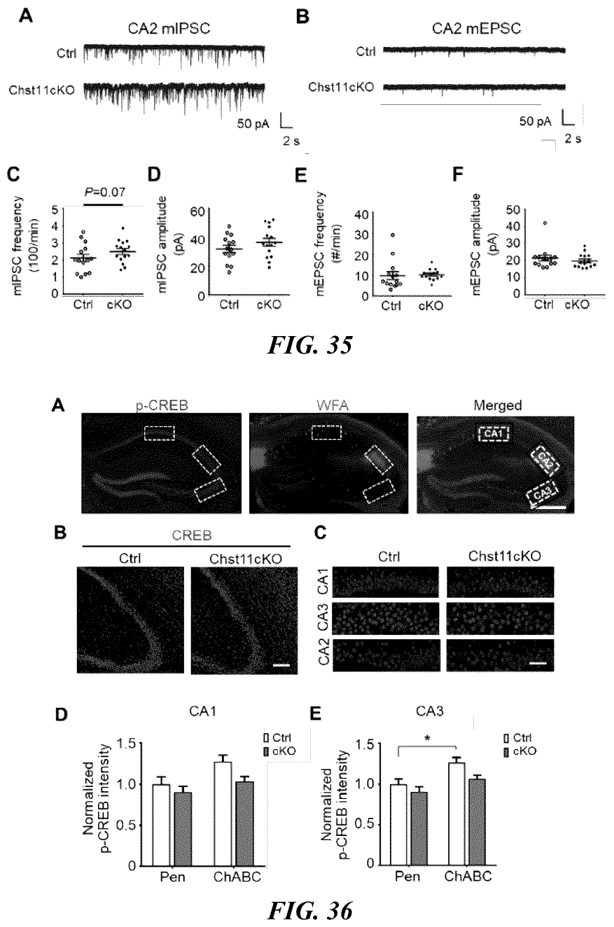

To assess whether the observed synaptic defects alter E/I synaptic transmission, we performed whole-cell patch clamp recordings on acute hippocampal slices from Chst11cKO and Ctrl mice. As reported previously, patch clamping of CA2 neurons is technically challenging due to the high PNN densities surrounding these neurons. Nonetheless, CA2 Chst11cKO neurons showed a trend toward increased frequency of miniature inhibitory postsynaptic currents (mIPSCs) compared to Ctrl neurons (P=0.07; FIG. 35, panels A and C), consistent with the increased number of inhibitory synapses observed in Chst11cKO neurons. The amplitude of mIPSC and miniature excitatory postsynaptic currents (mEPSCs) was similar for both CA2 Chst11cKO and Ctrl neurons (FIG. 35, panels D and F), and no significant change in mEPSC frequency was detected (FIG. 35, panels B and E). Our ability to measure decreases in mEPSC frequency may have been hindered by the low frequencies of CA2 pyramidal neurons. Overall, the data suggest that deletion of CS 4-O-sulfation alters dendritic spine maturation and E/I synapse ratios and leads to functional deficits in synaptic transmission.

### Example 12

### Loss of CS 4-O-Sulfation and Increased PNNs Reduce CREB Activity in Hippocampal Neurons

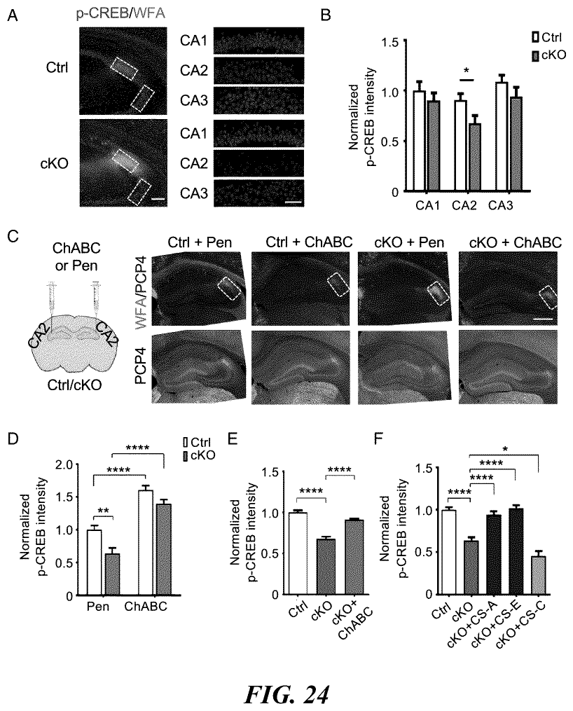

It was shown previously that neurotrophins such as brain-derived neurotrophic factor (BDNF) bind preferentially to 4-O-sulfated CS motifs and form ternary CS-BDNF-TrkB receptor complexes. Cell surface engineering of cultured hippocampal neurons to display CS polysaccharides enriched in the CS-A or CS-E motifs led to enhanced neurotrophin signaling and neurite outgrowth, suggesting that CS-A/E polysaccharides can function as coreceptors to activate neurotrophin signaling. On the other hand, CSPGs have also been shown to modulate BDNF-induced dendritic spine growth in cultured cortical neurons and TrkB phosphorylation in PV⁺ neurons in vitro via the PTPa receptor. We therefore hypothesized that the synaptic defects in Chst11cKO neurons might stem from dysregulation of BDNF signaling. To test this, we examined cyclic AMP-response element binding protein (CREB), an important regulator of synapse development and plasticity whose phosphorylation is induced by BDNF. Indeed, the average levels of CREB phosphorylated at Ser133 (p-CREB) were significantly reduced in the CA2 neurons of Chst11cKO mice compared to Ctrl mice, whereas total CREB levels remained unchanged (FIG. 24, panels A and B and FIG. 36, panels A-C). This reduction was specific to the area CA2, consistent with the marked change in PNN densities observed in this hippocampal subregion (FIG. 21, panel C). To assess whether the observed inhibition of CREB activity was due to the increase in PNN densities surrounding CA2 neurons, we stereotaxically delivered ChABC or penicillinase (Pen) as a control into the CA2 hippocampus of adult Chst11cKO and Ctrl mice. WFA staining after 2 wk revealed that the PNN levels of Chst11cKO mice injected with ChABC were comparable to those of Ctrl mice injected with Pen (FIG. 24, panel C). Importantly, ChABC-mediated pruning of PNNs in the CA2 significantly enhanced the p-CREB levels in Chst11cKO mice (FIG. 24, panel D). Interestingly, Ctrl mice injected with ChABC showed an increase in p-CREB levels in the CA2 (FIG. 24, panel D), suggesting that altering basal PNN levels in the brains of normal adult mice can modulate CREB activity.

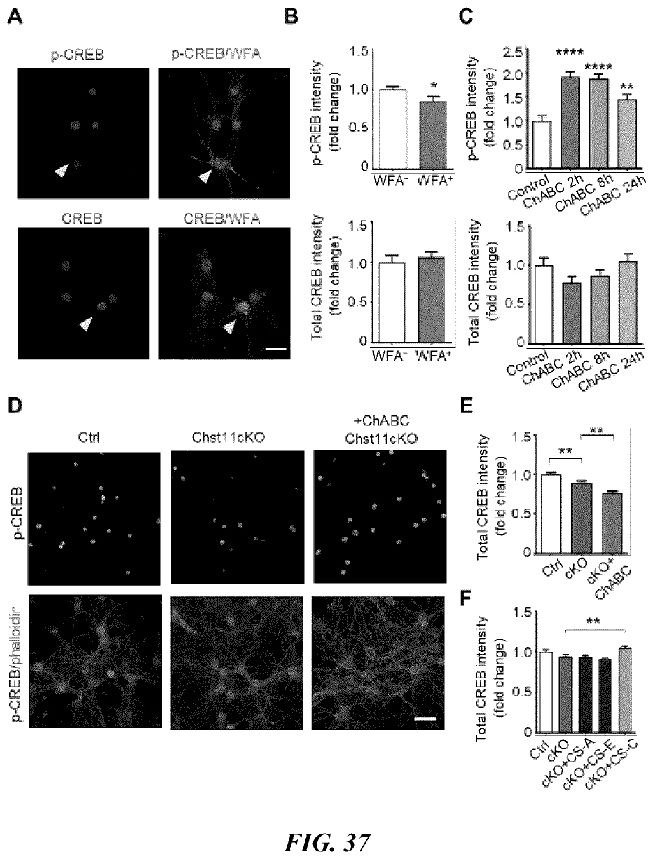

The regulation of p-CREB levels were also studied using hippocampal neuronal cultures. Neurons from wild-type C57 mice enwrapped by PNNs had lower p-CREB levels (FIG. 37, panels A and B), and PNN removal using ChABC significantly increased p-CREB levels, but not total CREB levels, for at least 24 h (FIG. 37, panel C). Consistent with the in vivo data, Chst11cKO neurons had more WFA⁺ PNNs (FIG. 32, panels B and C) and reduced p-CREB levels (FIG. 24, panel E and FIG. 37, panel D) compared to Ctrl neurons. Moreover, ChABC digestion of the PNNs surrounding Chst11cKO neurons led to a significant increase in p-CREB levels, returning them to Ctrl levels (FIG. 24, panel E). Notably, treatment of Chst11cKO neurons with exogenous natural polysaccharides enriched in the 4-O-sulfated CS-A or CS-E motifs, but not the 6-O-sulfated CS-C motif, restored both the PNN (FIG. 34, panels A and B) and p-CREB (FIG. 24, panel F) levels to Ctrl levels. Taken together, these data suggest that 4-O-sulfation of CS polysaccharides regulates the density of PNNs surrounding hippocampal neurons, thereby modulating CREB activity and synapse development.

### Example 13

### Loss of CS 4-O-Sulfation and High PNN Densities Impair Social Memory and Elevate Anxiety Levels

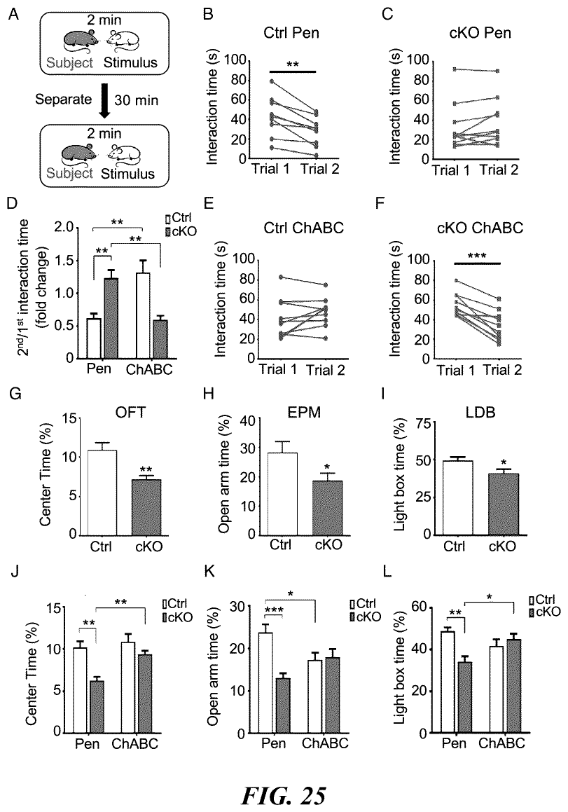

Neurological disorders such as autism, schizophrenia, and fragile X syndrome are characterized by structural alterations in dendritic spines and synapses. Moreover, mice with targeted CREB mutations or gene deletion exhibit deficiencies in long-term memory. As silencing of CA2 neurons disrupts social recognition memory, the effects of PNNs and CS 4-O-sulfation were examined on CA2-dependent social memory. Chst11cKO and Ctrl mice received bilateral stereotaxic injections of ChABC or Pen in the CA2 region, followed by a two-trial social memory test. In the first trial, the subject mouse was allowed to interact with a stimulus mouse for 2 min (FIG. 25, panel A). After 30 min of separation, the subject mouse was then reexposed in the second trial to the previously encountered stimulus mouse for 2 min. For Ctrl mice injected with Pen, the interaction time with the stimulus mouse was significantly reduced from the first trial to the second, indicative of normal social recognition memory (FIG. 25, panels B and D). In contrast, Chst11cKO mice injected with Pen exhibited no significant reduction in average interaction time between the two trials (FIG. 25, panels C and D). Importantly, Chst11cKO mice injected with ChABC showed a decrease in average interaction time during the second trial, suggesting that removal of the excess PNNs rescued the social memory deficits (FIG. 25, panels D and F). Interestingly, Ctrl mice injected with ChABC showed no reduction in interaction time from the first trial to the second (FIG. 25, panels D and E). Thus, while higher PNN densities in the area CA2 impair social memory, lower PNN densities caused by ChABC overdigestion also appear to be detrimental. These findings suggest that PNN levels are finely balanced in vivo under physiological conditions to achieve normal social memory function.

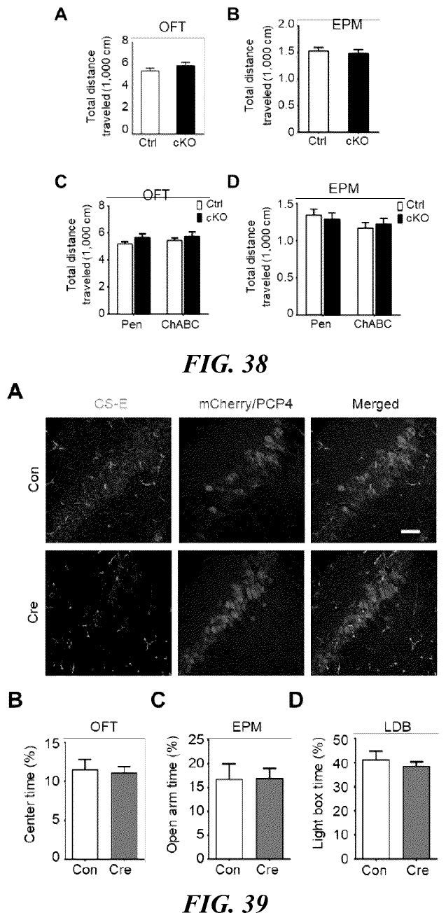

Next, we assessed whether loss of CS 4-O-sulfation in Chst11cKO mice was associated with other brain dysfunctions. Specifically, we monitored the anxiety level and general locomotor activity of Chst11cKO mice using the open-field test (OFT). While the total distance traveled by Chst11cKO mice was comparable to Ctrl mice (FIG. 38, panel A), Chst11cKO mice spent less time in the center of the open field arena relative to Ctrl mice (FIG. 25, panel G), suggesting a higher level of anxiety. To verify further the increased anxiety levels of these animals, we performed the elevated plus maze (EPM) and light-dark box (LDB) tests. Consistent with the enhanced anxiety levels shown in the OFT, Chst11cKO mice spent less time in the open arm of the EPM despite traveling comparable distances (FIG. 25, panel H and FIG. 38, panel B) and less time in the brightly lit chamber of the LDB compared to Ctrl mice (FIG. 25, panel I).

To probe whether PNNs contribute to the observed anxiety defects, we injected ChABC or Pen bilaterally into the CA2 region as before. Indeed, PNN digestion in the CA2 region of Chst11cKO mice decreased the higher anxiety levels, restoring them to similar levels as Pen- or ChABC-injected Ctrl mice (FIG. 25, panels J-L) without affecting locomotor activity (FIG. 38, panels C and D). Interestingly, contrary to what we observed for social memory (FIG. 25, panels D and E), no significant differences in anxiety-related OFT and LDB behavior were detected between Pen- and ChABC-injected Ctrl mice (FIG. 25, panels J and L), although a reduction in the open arm duration was observed in the EPM (FIG. 25, panel K). Thus, PNN digestion solely in the CA2 region of Ctrl mice appears to be sufficient to alter social recognition memory but not anxiety levels. Nonetheless, restoring PNN densities in the CA2 hippocampus of Chst11cKO mice to normal Ctrl levels is sufficient to rescue the observed anxiety-like behavior.

### Example 14

### Hippocampal-Specific Deletion of Chst11 Increases PNN Densities in the CA2 Region and Leads to Impaired Social Memory

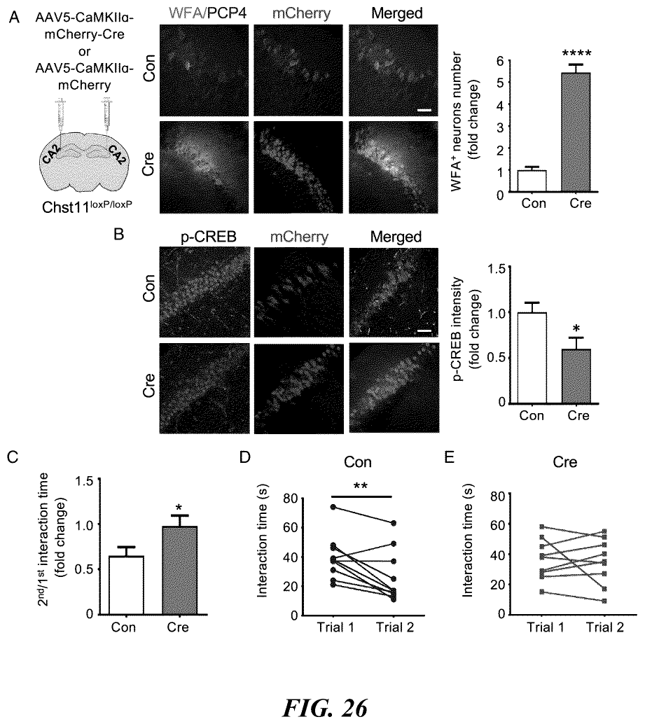

As previous studies have suggested that inactivation of CA2 pyramidal neurons does not influence anxiety-like behavior, the region-specific effects of Chst11 deletion was further explored. A Cre-recombinase-expressing virus was employed to knock out Chst11 with high regional and temporal specificity in the CA2 region. Adult Chst11^{loxP/loxP} mice were stereotaxically injected with viruses expressing either mCherry-tagged Cre recombinase (AAV5-CaMKIIα-mCherry-Cre, Cre) or only mCherry (AAV5-CaMKIIα-mCherry, Con) under the control of the calcium/calmodulin-dependent protein kinase II (CaMKII) promoter (FIG. 26, panel A). This restricted Cre expression and hence Chst11 gene deletion specifically to excitatory neurons in the CA2 hippocampus. Accordingly, we observed a robust mCherry signal in the CA2 region of Cre-injected Chst11^{loxP/loxP} mice three weeks after virus injection, indicative of region-specific Cre expression in this area (FIG. 26, panel A). The CA2-specific loss of CS 4-O-sulfation was confirmed in mCherry-positive neurons by immunostaining with an anti-CS-E antibody (FIG. 39, panel A). Similar to Chst11cKO mice, Cre-injected Chst11^{loxP/loxP} mice showed a significant increase in WFA⁺ PNN-enwrapped neurons (FIG. 26, panel A) and a reduction in p-CREB levels in the CA2 region compared to Con-injected mice (FIG. 26, panel B). These results demonstrate that modulation of CS 4-O-sulfation specifically in the CA2 region of adult mice is sufficient to alter PNN densities and CREB activity in the hippocampus.

Next, the social memory and anxiety-like behavior of these mice were examined. While Con-injected mice displayed a significant reduction in social interaction time in the second trial, Cre-injected mice spent an equal amount of time in both trials (FIG. 26, panels C-E), indicating that CA2-specific Chst11 deletion leads to social memory defects. Despite the increased anxiety-like behavior observed in Chst11cKO mice (FIG. 25, panels G-I), CA2-specific deletion of Chst11 at adulthood did not alter anxiety levels. Cre-injected Chst11^{loxP/loxP} mice showed comparable anxiety levels to Con-injected Chst11^{loxP/loxP} mice, as assessed by the OFT, EPM, and LDB tests (FIG. 39, panels B-D). Taken together with the earlier observations that ChABC treatment did not affect anxiety levels in the Ctrl mice (FIG. 25, panels J-L), these findings suggest that CS 4-O-sulfation may influence anxiety-like behaviors through complex mechanisms involving CA2 neurons and other brain regions. Nevertheless, the results indicate that CA2-specific deletion of CS 4-O-sulfation at adulthood is sufficient to drive malformation of PNNs and social memory dysfunction.

### Example 15

### Additional Consideration Related to Chondroitin 4-O-Sulfation

Examples 9-14 demonstrate that 4-O-sulfated CS, the dominant sulfation motif on CSPGs in the adult brain, plays a critical role in regulating PNN levels and E/I synapse balance in the adult hippocampus. Brain-wide deletion of chondroitin 4-O-sulfation led to elevated PNNs, particularly in the CA2 hippocampus, and thereby disrupted CREB activation, synapse development, and social memory in mice. Modulation of 4-O-sulfation levels via other methods, including viral-mediated gene deletion and chemical inhibitors of CS sulfotransferases recapitulated the effects of Chst11 gene knockout, while ChABC and exogenous 4-O-sulfated, but not 6-O-sulfated, CS polysaccharides rescued the effects. Collectively, the data demonstrate that CS 4-O-sulfation is essential for the proper functioning of the hippocampus and contributes to higher brain functions.

During mouse brain development, the proportion of chondroitin 4-O-sulfation on CSPGs progressively increases, while the proportion of chondroitin 6-O-sulfation decreases. The function of 6-O-sulfation on CSPGs has been studied in the developing VC, where it was tightly linked to PNNs surrounding PV-expressing GABAergic inhibitory interneurons. Transgenic mice overexpressing the 6-O-sulfotransferase C6ST-1 showed fewer PNNs enwrapping PV⁺ interneurons and prolonged ocular dominance plasticity, leading to the proposal that a low 4S/6S ratio regulates the functional maturation of PV-expressing interneurons and maintains plasticity in the VC. Based on these results, chondroitin 4-O-sulfation was inferred to be a molecular brake that inhibits ocular dominance plasticity at the end of the critical period. Consistent with an inhibitory role, 4-O-sulfated CS motifs are also up-regulated in the glial scar after CNS injury and limit axon regeneration and neuroplasticity. Knockdown of Chst11 in zebrafish led to loss of chondroitin 4-O-sulfation and enhanced regeneration after spinal cord injury. However, the precise functions of 4-O-sulfated structures such as CS-A, which represents about 90% of the total CS and is the most abundant glycosaminoglycan structure in the adult brain, are not well understood.

As presented herein, the roles of 4-O-sulfation in the adult uninjured brain was directly examined. Impaired dendritic spine maturation, an imbalance of EI synapses, together with elevated anxiety levels and social memory defects, in mice with a brain-specific deletion of the chondroitin 4-O-sulfotransferase Chst11 gene were observed. The findings presented herein indicate critical albeit nuanced roles for chondroitin 4-O-sulfation in the regulation of PNNs and neuroplasticity, with distinct roles depending on the cell type, developmental stage, and brain region. Thus, the sulfation patterns on CSPGs must be finely tuned in a cell-type-specific manner, both during critical periods of development and throughout adulthood.

It was found that CS 4-O-sulfation in PNNs modulated CREB activity important for excitatory synapse development and maturation in the CA2 hippocampus. Brain-wide ablation of 4-O-sulfation markedly increased PNNs and caused fewer mature dendritic spines in the CA2 region. Furthermore, a decrease in E/I synapse ratio was observed in hippocampal neurons cultured from mice lacking 4-O-sulfation. Previous studies have shown that 4-O-sulfated CS motifs presented on cell surfaces are specifically recognized by neurotrophins such as BDNF and can stimulate neurotrophin-mediated neurite growth. Moreover, BDNF-TrkB pathway activation and enhanced CREB activity promote dendritic spine maturation, while loss of BDNF via Cre-mediated bdnf deletion specifically in postmitotic neurons impairs dendritic spine growth. Thus, it was postulated that the observed synaptic defects might be caused by dysregulation of BDNF-CREB signaling. In accordance with this hypothesis, CREB activity was significantly reduced in CA2 neurons of Chst11cKO mice compared to Ctrl mice, and this activity was rescued by restoring PNN levels to normal Ctrl levels. Notably, the deficits in PNNs, CREB activation, and synapses could also be recapitulated by decreasing CS 4-O-sulfation levels in wild-type hippocampal neurons using a CS sulfotransferase chemical inhibitor. Moreover, restoration of CS 4-O-sulfation levels in Chst11cKO neurons using natural CS-A or CS-E polysaccharides reduced the PNN densities and returned CREB activity to normal levels. It is believed that the sulfation patterns on CSPGs in PNNs may regulate CREB activity and dendritic spine maturation via at least two distinct mechanisms. First, chondroitin 4-O-sulfation levels modulate the density of PNNs, which can act as “molecular sieves” to control diffusion and access of extracellular ligands such as BDNF to neuronal cell surfaces. Second, the presentation of 4-O-sulfated motifs on CSPGs within PNNs can act as “molecular scaffolds” for extracellular ligands, which facilitates the specific recruitment of BDNF and possibly other ligands to the surface of hippocampal neurons and assists in ligand-receptor activation. In support of these mechanisms, it was previously shown that natural CS-E polysaccharides are capable of forming ternary CS-BDNF-TrkB complexes and promoting both neurotrophin pathway activation and neurite outgrowth. In Chst11cKO mice, the increased PNN densities, combined with the loss of 4-O-sulfation motifs important for proper BDNF function, likely account for the observed reduction in CREB activity and synaptic defects. Overall, the present studies suggest that 4-O-sulfation of CSPGs plays a critical role in controlling PNN densities and maintaining optimal levels of ligand-receptor engagement at the cell surface, thereby regulating CREB activity essential for synapse development and hippocampal function. It is worth noting that additional proteins and cell types may also play a role. For example, semaphorin 3A has been shown to bind PNNs via 4-O-sulfated CS-E motifs and to promote dendritic spine development and the clustering of postsynaptic molecules. Moreover, Sema3F is a negative regulator of spine development, and the CSPG neurocan inhibited Sema3F-induced spine elimination. Microglia, astrocytes, and oligodendrocytes have also been shown to regulate dendritic spine development, and astrocytes are a known source of BDNF. Additional studies will be needed to fully elucidate the mechanisms contributing to the synaptic defects observed in Chst11cKO mice.

Emerging evidence suggests that PNNs contribute to cognitive functions in multiple brain regions, including cortical regions, the hippocampus, and the cerebellum. However, these conclusions were obtained by destroying PNNs using ChABC, and they raise another important question—do PNNs require optimal levels and precise timing to perform these specific functions? The present study demonstrates not only that ablation of CA2 PNNs impairs social memory but also that a CA2-specific and adult-specific increase of PNNs is sufficient to drive social memory dysfunction. It was found that social memory was impaired when PNN levels were either too high, as observed in Chst11cKO mice, or too low, as observed in ChABC-treated Ctrl mice. Consistent with these latter observations, PNN digestion in the area CA2 was recently reported to disrupt long-term depression of inhibitory transmission (iLTD) and social memory formation in mice. Notably, it was found that CA2 region-specific deletion of 4-O-sulfation was sufficient to cause malformation of PNNs and social memory deficits. Furthermore, the addition of natural CS-A or CS-E polysaccharides to cultured neurons deficient in CS 4-O-sulfation restored the PNN levels and EI synapse ratios back to normal. Together, these observations suggest that dynamic modulation of CS 4-O-sulfation may enable the fine-tuned control of PNNs and allow for the remediation of E/I imbalances and CA2-associated dysfunctions.

In addition to affecting social memory, the present studies suggest that chondroitin 4-O-sulfation and PNNs influence anxiety-related behaviors. Although Chst11cKO mice showed elevated anxiety levels, Chst11-floxed mice injected with Cre in the CA2 hippocampus did not exhibit anxiety defects, suggesting the involvement of other brain regions in anxiety regulation. Consistent with these observations, previous studies have reported that silencing of CA2 output affects social memory but not anxiety-like behaviors, spatial memory, or contextual memory. Multiple brain areas and their interconnections have been suggested to regulate anxiety such as the amygdala, medial prefrontal cortex, hypothalamus, and the ventral CA1 region of the hippocampus. As the Chst11 gene deletion in Chst11cKO mice occurred in all brain regions, the elevated anxiety levels of Chst11cKO mice are likely due to the loss of 4-O-sulfation in brain regions other than CA2. Interestingly, however, restoring PNN densities only in the CA2 hippocampus to normal Ctrl levels was sufficient to rescue the anxiety-like behaviors of Chst11cKO mice. These findings suggest that while the cause of anxiety can be attributed to loss of 4-O-sulfation in multiple brain regions, release of CA2 activity by PNN removal may activate certain brain circuits and thereby produce anxiolytic effects. Transgenic animals lacking 4-O-sulfation or other CS sulfation motifs may be valuable models in this regard and contribute to a deeper understanding of anxiety, autism, and other neuropsychiatric disorders.

Pathological alterations in PNNs and CS sulfation are associated with neurodegenerative, neurodevelopmental, and mental disorders characterized by changes in cognition, emotion, and memory loss. For example, PNN numbers were significantly reduced in individuals with schizophrenia, and altered CS sulfation patterns, particularly 4-O-sulfated motifs, were observed in the postmortem brains of human subjects with Alzheimer's disease, bipolar disorder, and schizophrenia. Abnormal PNN formation was also detected in the CA2 hippocampus of Rett syndrome (Mecp2-null) and autism (Black and Tan Brachyury (BTBR) T+ Itpr3tf/J strain) mouse models. Our studies reveal that CS 4-O-sulfation regulates PNNs in the adult hippocampus and contributes to the proper balance of excitatory and inhibitory synapses, as well as hippocampal cognitive abilities such as social memory. Importantly, the present study demonstrates that enzymatic or chemical manipulation of chondroitin 4-O-sulfation levels specifically in adulthood may allow for the dynamic modulation of PNN levels, remediation of synaptic FJI imbalances, and CA2-dependent social behaviors. Thus, treatments that target chondroitin 4-O-sulfation may represent a strategy to address diseases with synaptic disturbances in EI balance or PNN-associated pathologies, including autism, Rett syndrome, schizophrenia, and Alzheimer's disease. More broadly, the present studies identify an important role for the polysaccharides on CSPGs in the adult hippocampus and further highlight the importance of elucidating the roles of glycans in cognitive functions and neurological diseases.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A disaccharide, having a formula of: 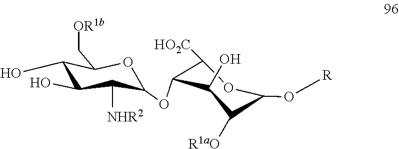
wherein R^{1a}, R^{1b} and R² are independently H, methyl, ethyl, acetyl (Ac), SO₃H, PO₂(OCH₃)H or PO₃H₂; and R is H, a C1-C12 1 R group linear or branched, substituted or unsubstituted aliphatic primary or secondary amine, a C1-C12 linear or branched, substituted or unsubstituted aliphatic primary or secondary amine.

2. The disaccharide of claim 1, wherein R is a fluorous tag.

3. The disaccharide of claim 1, wherein R^{1a} and/or R^{1b} is a hydroxyl protecting group.

4. The disaccharide of claim 1, wherein the disaccharide is N-sulfated and/or O-sulfated.

5. The disaccharide of claim 1, wherein R² is SO₃H, R^{1a} is SO₃H, and/or R^{1b} is SO₃H.

6. A method of synthesizing the polysaccharide of claim 1.

7. A polysaccharide, having a formula of: 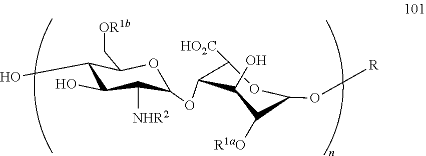
wherein n is any integer ranging from 2 to 200; R^{1a}, R^{1b} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine, a C1-C12 linear or branched, substituted or unsubstituted aliphatic primary or secondary amine.

8. The polysaccharide of claim 7, wherein R^{1a}, R^{1b} and/or R² is SO₃H.

9. A method for synthesizing the polysaccharide of claim 7.

10. (canceled)

11. The polysaccharide of claim 7, wherein n=2, and wherein R^{1a} and/or R^{1b} is a hydroxyl protecting group.

12. (canceled)

13. A method of treating a neurological disease or disorder in a subject in need thereof, comprising:
administrating to the subject a therapeutically effective amount of a composition comprising an effective amount of a 4-O-sulfated chondroitin sulfate polysaccharide, thereby treating the neurological disease or disorder in the subject.

14. The method of claim 13, wherein the 4-O-sulfated chondroitin sulfate polysaccharide has a general formula of: 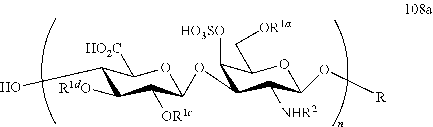
wherein n is any integer ranging from 2 to 200, preferably 20-200; R^{1a}, R^{1c}, R^{1d} and R² are independently H, methyl, ethyl, acetyl (Ac), SO₃H, PO₂(OCH₃)H or PO₃H₂; and R is H, a C1-C12 linear or branched, substituted or unsubstituted aliphatic primary or secondary amine.

15. The method of claim 13, wherein the 4-O-sulfated chondroitin sulfate polysaccharide has a general formula of: 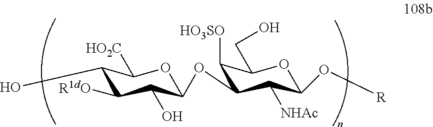
wherein R is H, a C1-C12 linear or branched, substituted or unsubstituted aliphatic primary or secondary amine; n is any integer ranging from 20 to 200.

16. The method of claim 13, wherein the 4-O-sulfated chondroitin sulfate polysaccharide has a general formula of: 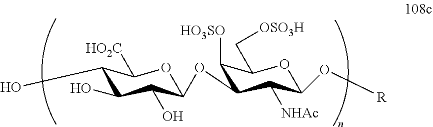
wherein R is H, a C1-C12 linear or branched, substituted or unsubstituted aliphatic primary or secondary amine; n is any integer ranging from 20 to 200.

17. The method of claim 13, wherein the neurological disease or disorder is selected from the group consisting of: Alzheimer's disease, bipolar disorder, schizophrenia, autism, fragile X syndrome, Rett syndrome, anxiety or anxiety-related disorder, social memory dysfunction, and a combination thereof.

18. The method of claim 13, wherein the subject is a mammal.

19. The method of claim 18, wherein the subject is a human.

20. The method of claim 13, wherein the composition is administered to the subject by oral administration or intravenous administration.
